(19) United States
(12) Patent Application Publication
(10) Pub. No.: US 2022/0275372 A1
(43) Pub. Date: Sep. 1, 2022

(54) CONJUGATE AND USES THEREOF

(71) Applicants: OXFORD UNIVERSITY INNOVATION LIMITED, Oxford (GB); UNITED KINGDOM RESEARCH AND INNOVATION, Swindon (GB); ASSOCIATION INSTITUT DE MYOLOGIE, Paris (FR); INSERM (INSTITUT NATONAL DE LA SANTE ET DE LA RECHERCHE MEDICALE), Paris (FR); SORBONNE UNIVERSITE, Paris (FR)

(72) Inventors: Matthew Wood, Oxford Oxfordshire (GB); Miguel Varela, Oxford Oxfordshire (GB); Ashling Holland, Oxford Oxfordshire (GB); Richard Raz, Oxford Oxfordshire (GB); Denis Furling, Paris (FR); Arnaud Klein, Paris (FR); Michael Gait, Cambridge Cambridgeshire (GB)

(73) Assignees: OXFORD UNIVERSITY INNOVATION LIMITED, Oxford (GB); UNITED KINGDOM RESEARCH AND INNOVATION, Swindon (GB); ASSOCIATION INSTITUT DE MYOLOGIE, Paris (FR); INSERM (INSTITUT NATONAL DE LA SANTE ET DE LA RECHERCHE MEDICALE), Paris (FR); SORBONNE UNIVERSITE, Paris (FR)

(21) Appl. No.: 17/631,654

(22) PCT Filed: Aug. 7, 2020
(86) PCT No.: PCT/GB2020/051891

§ 371 Date: Jan. 31, 2022

(30) Foreign Application Priority Data
Aug. 9, 2019 (GB) 1911403.2

(51) Int. Cl. C12N 15/113; A61K 47/64; A61P 21/00

(52) U.S. Cl. CPC C12N 15/1137; A61K 47/6455; A61P 21/00; C12N 2310/11

(57) ABSTRACT

The present invention relates to conjugates formed from a cell-penetrating peptide carrier linked to a therapeutic molecule, wherein the peptide carrier is defined by specific domains and the therapeutic molecule is a nucleic acid formed of trinucleotide repeats. The present invention further relates to the use of such a conjugate in methods of treatment or as a medicament, especially in the treatment of trinucleotide repeat disorders such as myotonic dystrophy (DM1).

### TECHNICAL FIELD

The present invention relates to a conjugate of a peptide carrier with a therapeutic molecule, wherein the peptide carrier is defined by specific domains and the therapeutic molecule is a nucleic acid formed of trinucleotide repeats. The present invention further relates to the use of such a conjugate in methods of treatment or as a medicament, especially in the treatment of trinucleotide repeat disorders such as myotonic dystrophy (DM1).

### BACKGROUND

Nucleic acid therapeutics are genomic medicines with the potential to transform human healthcare. Research has indicated that such therapeutics could have applications across a broad range of disease areas. In particular, the application of antisense oligonucleotide-based methods to modulate mRNA expression has become a desirable means of therapy at the forefront of precision medicine.

However, therapeutic development of these promising antisense therapeutics has been hampered by insufficient cell-penetrance and poor distribution characteristics.

Therefore there is a strong and urgent need to improve the delivery of antisense oligonucleotides in order to provide a more effective therapy for genetic diseases such as devastating trinucleotide repeat disorders.

Trinucleotide repeat disorders are genetic diseases characterised by the presence of an abnormally high number of repeats of a specific sequence of three nucleotides within genomic DNA, otherwise known as a trinucleotide repeat expansion. Trinucleotide repeat expansions are a specific type of microsatellite repeat, often known as microsatellite expansions. Typically, there is a threshold number of repeats that are found in a normal healthy subject, and if this number is exceeded then the disease is pathogenic. The threshold number differs between diseases and affected genes. It is also typical in these diseases that the number of repeats can indicate the severity of the disease. Generally, a higher number of repeats indicates a more severe presentation of the disease. The number of repeats can also be used to predict the age of onset of the diseases, with higher numbers of repeats indicating early onset.

At present, there are 14 known trinucleotide repeat disorders that affect humans. These disorders can be grouped by several methods, for example by where the trinucleotide repeat is located in the gene, whether it is in a protein coding ORF; in an exon; or in an untranslated region. Alternatively, they can be grouped by the sequence of the triplet repeat. In many trinucleotide disorders, the triplet repeat is ‘CAG’ and encodes glutamine, this group of disorders are commonly known as polyglutamine disorders. However, trinucleotide repeats having other sequences are known, and can be grouped as non-polyglutamine repeat disorders.

One trinucleotide disorder known as a non-polyglutamine repeat disorder is myotonic dystrophy type 1 (DM1). DM1 is caused by a trinucleotide repeat of ‘CTG’ present within the 3′ UTR of the DMPK gene. A normal number of repeats for this gene is between 5 and 34 repeats. Above 34 repeats, there may be some symptoms of the disease, and above 50 repeats the disease is pathogenic.

DM1, and other trinucleotide repeat disorders, typically affect the neuromuscular system and do not currently have any effective treatments.

Whilst the use of antisense oligonucleotides which can bind to repeat regions and interrupt splicing or translation has been theoretically proposed and shown in vitro, the use of such antisense oligonucleotides as therapeutics has not been possible due to the difficulty of delivering these molecules into affected cells. This is the case for the treatment of a wide variety of genetic diseases, including trinucleotide repeat disorders.

The use of viruses as delivery vehicles has been suggested, however their use is limited due to the immunotoxicity of the viral coat protein and potential oncogenic effects. Alternatively, a range of non-viral delivery vectors have been developed, amongst which peptides have shown the most promise due to their small size, targeting specificity and ability of trans-capillary delivery of large bio-cargoes. Several peptides have been reported for their ability to permeate cells either alone or carrying a bio-cargo.

For several years, cell-penetrating peptides have been conjugated to antisense oligonucleotides (in particular charge neutral phosphorodiamidate morpholino oligomers (PMO) and peptide nucleic acids (PNA)) in order to enhance the cell delivery of such oligonucleotide analogues by effectively carrying them across cell membranes to reach their pre-mRNA target sites in the cell nucleus. It has been shown that PMO therapeutics conjugated to certain arginine-rich peptides (known as P-PMOs or peptide-PMOs) can penetrate effectively into relevant cells.

In particular, PNA/PMO internalization peptides (Pips) have been developed which are arginine-rich CPPs that are comprised of two arginine-rich sequences separated by a central short hydrophobic sequence. These ‘Pip’ peptides were designed to improve serum stability whilst maintaining a high level of exon skipping, initially by attachment to a PNA cargo. Further derivatives of these peptides were designed as conjugates of PMOs, which were shown to lead to body-wide skeletal muscle therapy in DMD models, and importantly also including the heart, following systemic administration in mice.

Despite these carrier peptides being efficacious, their therapeutic application has been hampered by their associated toxicity.

Alternative carrier peptides having a single arginine rich domain such as R6Gly have also been produced. These peptides have been used to produce peptide conjugates with antisense oligonucleotides that have reduced toxicities, but these conjugates exhibited low efficacy in comparison to the Pip peptides.

Furthermore, almost all development of carrier peptides has been in the context of treating DMD. Peptides with a hydrophobic core domain have been proved to be especially active in the context of DMD. No research has yet been done into the use of such carrier peptides in other neuromuscular diseases having different causes and different pathologies.

Accordingly, the currently available carrier peptides have not yet been demonstrated as suitable for use in conjugates with nucleic acid therapeutics for treatment of genetic disorders, especially not diseases resulting from a different pathology such as trinucleotide repeat disorders.

The challenge in the field of carrier peptide technology has been to de-couple efficacy and toxicity. The present inventors have now identified, synthesized and tested conjugates comprising improved carrier peptides having a particular structure, covalently linked to a therapeutic nucleic acid for the treatment of a trinucleotide disorder which addresses at least this problem.

Statements of Invention

According to a first aspect of the present invention, there is provided a conjugate comprising: a peptide carrier covalently linked to a therapeutic molecule;

wherein the peptide carrier has a total length of 40 amino acids or less and comprises: two or more cationic domains each comprising at least 4 amino acid residues and one or more hydrophobic domains each comprising at least 3 amino acid residues, wherein the peptide carrier does not contain artificial amino acid residues;

and wherein the therapeutic molecule comprises a nucleic acid, wherein the nucleic acid comprises a plurality of trinucleotide repeats.

According to a second aspect of the present invention, there is provided a conjugate according to the first aspect for use as a medicament

According to a third aspect of the present invention, there is provided a method of treatment of a disease in a subject, the method comprising: administering an effective amount of the conjugate according to the first aspect to the subject.

According to a fourth aspect of the present invention, there is provided a conjugate according to the first aspect for use in the prevention or treatment of a trinucleotide repeat disorder.

According to a fifth aspect of the present invention, there is provided a method of prevention or treatment of a trinucleotide repeat disorder in a subject, the method comprising: administering an effective amount of the conjugate according to the first aspect to the subject.

According to a sixth aspect of the present invention, there is provided a pharmaceutical composition comprising a conjugate according to the first aspect.

In one embodiment of the second, third, fourth or fifth aspects, the conjugate is comprised in a pharmaceutical composition.

Further features and embodiments of the invention will now be described in the following headed sections. Unless explicitly noted otherwise, any feature may be combined with the above aspects, or with other features herein, in any compatible combination. Individual features are not limited to any particular embodiment. The section headings used herein are for organisational purposes only and are not to be construed as limiting the subject matter described.

References to a ‘peptide carrier’ throughout denote a peptide which is suitable to transport a molecule which is conjugated thereto into cells i.e. a cell-penetrating peptides. The terms ‘cell penetrating peptide’ and ‘peptide carrier’ and ‘peptide’ may used interchangeably throughout.

References to ‘X’ throughout denote any form of the artificial, synthetically produced amino acid aminohexanoic acid.

References to ‘B’ throughout denote the natural but non-genetically encoded amino acid beta-alanine.

References to ‘Ac’ throughout denote acetylation of the relevant peptide.

References to ‘Hyp’ throughout denote the natural but non-genetically encoded amino acid hydroxyproline.

References to other capital letters throughout denote the relevant genetically encoded amino acid residue in accordance with the accepted alphabetic amino acid code.

References to an ‘artificial’ amino acid or residue herein denotes any amino acid that does not occur in nature and includes synthetic amino acids, modified amino acids (such as those modified with sugars), non-natural amino acids, man-made amino acids, spacers, and non-peptide bonded spacers. For the avoidance of doubt, aminohexanoic acid (X) is an artificial amino acid in the context of the present invention. For the avoidance of doubt, beta-alanine (B) and hydroxyproline (Hyp) occur in nature and therefore are not artificial amino acids in the context of the present invention but are natural amino acids. Artificial amino acids may include, for example, 6-aminohexanoic acid (X), tetrahydroisoquinoline-3-carboxylic acid (TIC), 1-(amino)cyclohexanecarboxylic acid (Cy), and 3-azetidine-carboxylic acid (Az), 11-aminoundecanoic acid.

References to ‘cationic’ herein denote an amino acid or domain of amino acids having an overall positive charge at physiological pH.

By ‘arginine rich’ or ‘histidine rich’ it is meant that at least 40% of the cationic domain is formed of said residue/s.

References to ‘hydrophobic’ herein denote an amino acid or domain of amino acids having the ability to repel water or which do not mix with water.

### DETAILED DESCRIPTION

The present invention is based on the finding that the attachment of particular peptide carriers to a nucleic acid which is suitable for preventing and treating trinucleotide repeat disorders, allows the nucleic acid to effectively penetrate target cells and bind to target trinucleotide repeat expansions present in genes of affected subjects. This activity reduces the levels of repeat expansion transcripts and/or proteins present in a cell, and thereby blocks their pathological interaction with the splicing machinery of the cell, normalising splicing and improving the physiological condition of said subjects.

Advantageously, the peptide carriers described herein seems to increase the ability of the therapeutic nucleic acid to resist degradation, penetrate target cells, and reach the target trinucleotide expansions to provide therapy. In addition, the conjugates of the invention have much lower toxicity than conjugates formed with known peptide carriers. Therefore, the conjugate provides a means for effective delivery of a nucleic acid therapy for trinucleotide repeat disorders whilst remaining non-toxic to the subject.

The inventors believe that is the first time any peptide carriers with a hydrophobic core have been shown to be effective in the treatment of neuromuscular diseases outside the context of DMD. Prior research has focussed on using peptide carriers for delivery of therapeutics to treat DMD. The pathology of DMD compared with the pathology of trinucleotide repeat disorders is quite different. In particular DMD involves active muscle degeneration and muscle turnover and repair including inflammation whereas a trinucleotide repeat disorder such as myotonic dystrophy type 1 (DM1) involves muscle dysfunction without overt degeneration. It is believed by the present inventors that the peptide carriers interact with muscle membranes to allow efficient delivery of the therapeutic molecule, and therefore the types of membranes that they are interacting with vary greatly between degenerative muscle and non-degenerative muscle i.e. between DMD and trinucleotide repeat disorders. Contrary to degenerative diseases such as DMD, in DM1 the muscle membrane is not disrupted, and therefore it was expected that conjugate penetration into the muscle tissue would be inhibited and far more difficult to achieve. However, based on the data presented herein, the peptide carriers have not only been shown for the first time to be effective in delivery to non-degenerative muscle for treating DM1, but unexpectedly shown to work more efficiently for DM1 than DMD.

In the presented data herein, the conjugates of the invention maintain good levels of efficacy and delivery to key target tissues that are affected by trinucleotide disorders such as the gastrocnemius and quadriceps skeletal muscles. Furthermore, these conjugates demonstrate an improvement in efficacy compared with previously available carrier peptides when used in the same conjugate. The conjugates of the invention target mutant CUGexpanded-DMPK transcripts to prevent the formation of nuclear foci and thereby prevent the detrimental sequestration of MBNL1 splicing factor by the nuclear RNA foci, and consequently mitigate MBNL1 functional loss which is responsible for splicing defects in multiple genes and muscle dysfunction.

This is demonstrated herein by the reduction in the number of nuclear foci formed by DMPK transcripts containing the expansion after administration of a conjugate of the invention, and splicing correction of genes after administration of a conjugate of the invention, which genes are typically misspliced in DM1 due to the reduced availability of MBNL1 sequestered by the trinucleotide repeat expansion transcripts. Specifically, the conjugates demonstrated herein show a 50-90% splicing correction towards healthy controls excluding clicnl exon 7a and mblnl1 exon5, and including serca exon22 when comparing to untreated cells/subjects. This is further demonstrated by an improvement in the physiological condition of trinucleotide disorders, as is shown herein in DM1 models where myotonia in mice was normalised and corrected to the point of complete recovery even after a single injection of the conjugates described herein.

Surprisingly, the inventors have found that the peptide carriers used in the conjugate deliver the therapeutic molecule effectively into the nuclear compartment, and into the nuclear aggregates of DMPK transcripts at sufficient concentration to allow a favourable stoichiometric interaction with the CUG mutation.

At the same time, the conjugates of the invention act effectively in vivo with reduced clinical signs following systemic injection and lower toxicity as observed through measurement of biochemical markers. Crucially, the present conjugates are demonstrated to show a surprisingly reduced toxicity following similar systemic injection into mice when compared with previous carrier peptides in the same conjugate. As is demonstrated herein, the conjugates of the invention cause no significant increase in toxicity markers compared to saline at doses that are therapeutically relevant, and maintain cell viability whilst conjugates using prior peptide carriers show significant cell mortality. When the conjugates are administered to mice, the mice have a quick recovery time which is much faster than after administration of conjugates formed with previously available peptides.

Accordingly, the conjugates of the invention offer improved suitability for use as a safe and effective therapy for trinucleotide repeat disorders in humans, providing an avenue for treatment of these devastating diseases that have otherwise been untreatable.

Artificial Amino Acids

The present invention relates to conjugates comprising carrier peptides that have a particular structure in which there are no artificial amino acid residues.

Suitably, the peptide does not contain aminohexanoic acid residues. Suitably, the peptide does not contain any form of aminohexanoic acid residues. Suitably, the peptide does not contain 6-aminohexanoic acid residues.

Suitably, the peptide contains only natural amino acid residues, and therefore consists of natural amino acid residues.

Suitably, artificial amino acids such as 6-aminohexanoic acid that are typically used in cell-penetrating peptides are replaced by natural amino acids. Suitably the artificial amino acids such as 6-aminohexanoic acid that are typically used in cell-penetrating peptides are replaced by amino acids selected from beta-alanine, serine, proline, arginine and histidine or hydroxyproline.

In one embodiment, aminohexanoic acid is replaced by beta-alanine. Suitably, 6-aminohexanoic acid is replaced by beta-alanine.

In one embodiment, aminohexanoic acid is replaced by histidine. Suitably, 6-aminohexanoic acid is replaced by histidine.

In one embodiment, aminohexanoic acid is replaced by hydroxyproline. Suitably, 6-aminohexanoic acid is replaced by hydroxyproline.

Suitably, the artificial amino acids such as 6-aminohexanoic acid that are typically used in cell-penetrating peptides may be replaced by a combination of any of beta-alanine, serine, proline, arginine and histidine or hydroxyproline, suitably a combination of any of beta-alanine, histidine, and hydroxyproline.

In one embodiment, the peptide carrier may have a total length of 40 amino acid residues or less, the peptide comprising:

two or more cationic domains each comprising at least 4 amino acid residues; and one or more hydrophobic domains each comprising at least 3 amino acid residues; wherein at least one cationic domain comprises histidine residues.

Suitably, wherein at least one cationic domain is histidine rich.

Suitably, what is meant by histidine rich is defined herein in relation to the cationic domains.

Cationic Domain

The present invention relates to conjugates comprising short peptide carriers having a particular structure in which there are at least two cationic domains having a certain length.

Suitably, the peptide comprises up to 4 cationic domains, up to 3 cationic domains.

Suitably, the peptide comprises 2 cationic domains.

As defined above, the peptide comprises two or more cationic domains each having a length of at least 4 amino acid residues.

Suitably, each cationic domain has a length of between 4 to 12 amino acid residues, suitably a length of between 4 to 7 amino acid residues.

Suitably, each cationic domain has a length of 4, 5, 6, or 7 amino acid residues.

Suitably, each cationic domain is of similar length, suitably each cationic domain is the same length.

Suitably, each cationic domain comprises cationic amino acids and may also contain polar and or nonpolar amino acids.

Non-polar amino acids may be selected from: alanine, beta-alanine, proline, glycine, cysteine, valine, leucine, isoleucine, methionine, tryptophan, phenylalanine. Suitably non-polar amino acids do not have a charge.

Polar amino acids may be selected from: serine, asparagine, hydroxyproline, histidine, arginine, threonine, tyrosine, glutamine. Suitably, the selected polar amino acids do not have a negative charge.

Cationic amino acids may be selected from: arginine, histidine, lysine. Suitably, cationic amino acids have a positive charge at physiological pH.

Suitably each cationic domain does not comprise anionic or negatively charged amino acid residues.

Suitably each cationic domain comprises arginine, histidine, beta-alanine, hydroxyproline and/or serine residues.

Suitably each cationic domain consists of arginine, histidine, beta-alanine, hydroxyproline and/or serine residues.

Suitably, each cationic domain comprises at least 40%, at least 45%, at least 50% cationic amino acids.

Suitably, each cationic domain comprises a majority of cationic amino acids. Suitably, each cationic domain comprises at least 55%, at least 60%, at least 65% at least 70%, at least 75%, at least 80%, at least 85%, at least 90%, at least 95% cationic amino acids.

Suitably, each cationic domain comprises an isoelectric point (p1) of at least 7.5, at least 8.0, at least 8.5, at least 9.0, at least 9.5, at least 10.0, at least 10.5, at least 11.0, at least 11.5, at least 12.0.

Suitably, each cationic domain comprises an isoelectric point (p1) of at least 10.0.

Suitably, each cationic domain comprises an isoelectric point (p1) of between 10.0 and 13.0

In one embodiment, each cationic domain comprises an isoelectric point (p1) of between 10.4 and 12.5.

Suitably the isoelectric point of a cationic domain is calculated at physiological pH by any suitable means available in the art. Suitably, by using the IPC (www.isoelectric.org) a web-based algorithm developed by Lukasz Kozlowski, Biol Direct. 2016; 11: 55. DOI: 10.1186/s13062-016-0159-9.

Suitably, each cationic domain comprises at least 40%, at least 45%, at least 50%, at least 55%, at least 60%, at least 60%, at least 65%, least 70% arginine and/or histidine residues.

Suitably, a cationic domain may comprise at least 40%, at least 45%, at least 50%, at least 55%, at least 60%, at least 60%, at least 65%, least 70% arginine residues.

Suitably, a cationic domain may comprise at least 40%, at least 45%, at least 50%, at least 55%, at least 60%, at least 60%, at least 65%, least 70% histidine residues.

Suitably, a cationic domain may comprise a total of between 1-5 histidine and 1-5 arginine residues. Suitably, a cationic domain may comprise between 1-5 arginine residues. Suitably, a cationic domain may comprise between 1-5 histidine residues. Suitably, a cationic domain may comprise a total of between 2-5 histidine and 3-5 arginine residues. Suitably, a cationic domain may comprise between 3-5 arginine residues. Suitably, a cationic domain may comprise between 2-5 histidine residues.

Suitably, each cationic domain comprises one or more beta-alanine residues. Suitably, each cationic domain may comprise a total of between 2-5 beta-alanine residues, suitably a total of 2 or 3 beta-alanine residues.

Suitably, a cationic domain may comprise one or more hydroxyproline residues or serine residues.

Suitably, a cationic domain may comprise between 1-2 hydroxyproline residues. Suitably a cationic domain may comprise between 1-2 serine residues.

Suitably all of the cationic amino acids in a given cationic domain may be histidine, alternatively, suitably all of the cationic amino acids in a given cationic domain may be arginine.

Suitably, the peptide may comprise at least one histidine rich cationic domain. Suitably, the peptide may comprise at least one arginine rich cationic domain.

Suitably, the peptide may comprise at least one arginine rich cationic domain and at least one histidine rich cationic domain.

In one embodiment, the peptide comprises two arginine rich cationic domains.

In one embodiment, the peptide comprises two histidine rich cationic domains.

In one embodiment, the peptide comprises two arginine and histidine rich cationic domains.

In one embodiment, the peptide comprises one arginine rich cationic domain and one histidine rich cationic domain.

Suitably, each cationic domain comprises no more than 3 contiguous arginine residues, suitably no more than 2 contiguous arginine residues.

Suitably, each cationic domain comprises no contiguous histidine residues.

Suitably, each cationic domain comprises arginine, histidine and/or beta-alanine residues. Suitably, each cationic domain comprises a majority of arginine, histidine and/or beta-alanine residues. Suitably, at least 70%, at least 75%, at least 80%, at least 85%, at least 90%, at least 95%, 100% of the amino acid residues in each cationic domain are arginine, histidine and/or beta-alanine residues. Suitably, each cationic domain consists of arginine, histidine and/or beta-alanine residues.

In one embodiment, the peptide comprises a first cationic domain comprising arginine and beta-alanine residues and a second cationic domain comprising arginine and beta-alanine residues.

In one embodiment, the peptide comprises a first cationic domain comprising arginine and beta-alanine resides, and a second cationic domain comprising histidine, beta-alanine, and optionally arginine residues.

In one embodiment, the peptide comprises a first cationic domain comprising arginine and beta-alanine resides, and a second cationic domain comprising histidine and beta-alanine residues.

In one embodiment, the peptide comprises a first cationic domain consisting of arginine and beta-alanine residues and a second cationic domain consisting of arginine and beta-alanine residues.

In one embodiment, the peptide comprises a first cationic domain consisting of arginine and beta-alanine residues and a second cationic domain consisting of arginine, histidine and beta-alanine residues.

Suitably, the peptide comprises at least two cationic domains, suitably these cationic domains form the arms of the peptide. Suitably, the cationic domains are located at the N and C terminus of the peptide. Suitably therefore, the cationic domains may be known as the cationic arm domains.

In one embodiment, the peptide comprises two cationic domains, wherein one is located at the N-terminus of the peptide and one is located at the C-terminus of the peptide. Suitably at either end of the peptide. Suitably no further amino acids or domains are present at the N-terminus and C-terminus of the peptide, with the exception of other groups such as a terminal modification, linker and/or therapeutic molecule. For the avoidance of doubt, such other groups may be present in addition to ‘the peptide’ described and claimed herein. Suitably therefore each cationic domain forms the terminus of the peptide. Suitably, this does not preclude the presence of a further linker group as described herein.

Suitably, the peptide may comprise up to 4 cationic domains. Suitably, the peptide comprises two cationic domains.

In one embodiment, the peptide comprises two cationic domains that are both arginine rich.

In one embodiment, the peptide comprises one cationic domain that is arginine rich.

In one embodiment, the peptide comprises two cationic domains that are both arginine and histidine rich.

In one embodiment, the peptide comprises one cationic domain that is arginine rich and one cationic domain that is histidine rich.

Suitably, the cationic domains comprise amino acid units selected from the following: R, H, B, RR, HH, BB, RH, HR, RB, BR, HB, BH, RBR, RBB, BRR, BBR, BRB, RBH, RHB, HRB, BRH, HRR, RRH, HRH, HBB, BBH, RHR, BHB, HBH, or any combination thereof.

Suitably a cationic domain may also include serine, proline and/or hydroxyproline residues. Suitably the cationic domains may further comprise amino acid units selected from the following: RP, PR, RPR, RRP, PRR, PRP, Hyp; R[Hyp]R, RR[Hyp], [Hyp]RR, [Hyp]R[Hyp], [Hyp][Hyp]R, R[Hyp][Hyp], SB, BS, or any combination thereof, or any combination with the above listed amino acid units.

Suitably, each cationic domain comprises any of the following sequences: RBRRBRR (SEQ ID NO:1), RBRBR (SEQ ID NO:2), RBRR (SEQ ID NO:3), RBRRBR (SEQ ID NO:4), RRBRBR (SEQ ID NO:5), RBRRB (SEQ ID NO:6), BRBR (SEQ ID NO:7), RBHBH (SEQ ID NO:8), HBHBR (SEQ ID NO:9), RBRHBHR (SEQ ID NO:10), RBRBBHR (SEQ ID NO:11), RBRRBH (SEQ ID NO:12), HBRRBR (SEQ ID NO:13), HBHBH (SEQ ID NO:14), BHBH (SEQ ID NO:15), BRBSB (SEQ ID NO:16), BRB[Hyp]B (SEQ ID NO:17), R[Hyp]H[Hyp]HB (SEQ ID NO:18), R[Hyp]RR[Hyp]R (SEQ ID NO:19) or any combination thereof.

Suitably, each cationic domain consists any of the following sequences: RBRRBRR (SEQ ID NO:1), RBRBR (SEQ ID NO:2), RBRR (SEQ ID NO:3), RBRRBR (SEQ ID NO:4), RRBRBR (SEQ ID NO:5), RBRRB (SEQ ID NO:6), BRBR (SEQ ID NO:7), RBHBH (SEQ ID NO:8), HBHBR (SEQ ID NO:9), RBRHBHR (SEQ ID NO:10), RBRBBHR (SEQ ID NO:11), RBRRBH (SEQ ID NO:12), HBRRBR (SEQ ID NO:13), HBHBH (SEQ ID NO:14), BHBH (SEQ ID NO:15), BRBSB (SEQ ID NO:16), BRB[Hyp]B, R[Hyp]H[Hyp]HB, R[Hyp]RR[Hyp]R (SEQ ID NO:19) or any combination thereof.

Suitably, each cationic domain consists of one of the following sequences: RBRRBRR (SEQ ID NO:1), RBRBR (SEQ ID NO:2), RBRRBR (SEQ ID NO:4), BRBR (SEQ ID NO:7), RBHBH (SEQ ID NO:8), HBHBR (SEQ ID NO:9).

Suitably each cationic domain in the peptide may be identical or different. Suitably each cationic domain in the peptide is different.

Hydrophobic Domain

The present invention relates to conjugates comprising a short peptide carrier having a particular structure in which there is at least one hydrophobic domain having a certain length.

Suitably the peptide comprises up to 3 hydrophobic domains, up to 2 hydrophobic domains.

Suitably the peptide comprises 1 hydrophobic domain.

As defined above, the peptide comprises one or more hydrophobic domains each having a length of at least 3 amino acid residues.

Suitably, each hydrophobic domain has a length of between 3-6 amino acids. Suitably, each hydrophobic domain has a length of 5 amino acids.

Suitably, each hydrophobic domain may comprise nonpolar, polar, and hydrophobic amino acid residues.

Hydrophobic amino acid residues may be selected from: alanine, valine, leucine, isoleucine, phenylalanine, tyrosine, methionine, and tryptophan.

Non-polar amino acid residues may be selected from: proline, glycine, cysteine, alanine, valine, leucine, isoleucine, tryptophan, phenylalanine, methionine.

Polar amino acid residues may be selected from: Serine, Asparagine, hydroxyproline, histidine, arginine, threonine, tyrosine, glutamine.

Suitably the hydrophobic domains do not comprise hydrophilic amino acid residues.

Suitably, each hydrophobic domain comprises a majority of hydrophobic amino acid residues. Suitably, each hydrophobic domain comprises at least 70%, at least 75%, at least 80%, at least 85%, at least 90%, at least 95%, 100% hydrophobic amino acids. Suitably, each hydrophobic domain consists of hydrophobic amino acid residues.

Suitably, each hydrophobic domain comprises a hydrophobicity of at least 0.3,at least 0.4, at least 0.5, at least 0.6, at least 0.7, at least 0.8, at least 0.8, at least 1.0, at least 1.1, at least 1.2, at least 1.3.

Suitably, each hydrophobic domain comprises a hydrophobicity of at least 0.3, at least 0.35, at least 0.4, at least 0.45.

Suitably, each hydrophobic domain comprises a hydrophobicity of at least 1.2, at least 1.25, at least 1.3, at least 1.35.

Suitably, each hydrophobic domain comprises a hydrophobicity of between 0.4 and 1.4

In one embodiment, each hydrophobic domain comprises of a hydrophobicity of between 0.45 and 0.48.

In one embodiment, each hydrophobic domain comprises a hydrophobicity of between 1.27 and 1.39

Suitably, hydrophobicity is as measured by White and Wimley: W. C. Wimley and S. H. White, “Experimentally determined hydrophobicity scale for proteins at membrane interfaces” Nature Struct Biol 3:842 (1996).

Suitably, each hydrophobic domain comprises at least 3, at least 4 hydrophobic amino acid residues.

Suitably, each hydrophobic domain comprises phenylalanine, leucine, Isoleucine, tyrosine, tryptophan, proline, and glutamine residues. Suitably, each hydrophobic domain consists of phenylalanine, leucine, isoleucine, tyrosine, tryptophan, proline, and/or glutamine residues.

In one embodiment, each hydrophobic domain consists of phenylalanine, leucine, isoleucine, tyrosine and/or glutamine residues.

In one embodiment, each hydrophobic domain consists of tryptophan and/or proline residues.

Suitably, the peptide comprises one hydrophobic domain. Suitably the or each hydrophobic domain is located in the centre of the peptide. Suitably, therefore, the hydrophobic domain may be known as a core hydrophobic domain. Suitably, the or each hydrophobic core domain is flanked on either side by an arm domain. Suitably the arm domains may comprise one or more cationic domains and one or more further hydrophobic domains. Suitably, each arm domain comprises a cationic domain.

In one embodiment, the peptide comprises two arm domains flanking a hydrophobic core domain, wherein each arm domain comprises a cationic domain.

In one embodiment, the peptide consists of two cationic arm domains flanking a hydrophobic core domain.

Suitably the or each hydrophobic domain comprises one of the following sequences: YQFLI (SEQ ID NO:20), FQILY (SEQ ID NO:21), ILFQY (SEQ ID NO:22), FQIY (SEQ ID NO:23), WWW, WWPWW (SEQ ID NO:24), WPWW (SEQ ID NO:25), WWPW (SEQ ID NO:26) or any combination thereof.

Suitably the or each hydrophobic domain consists of one of the following sequences: YQFLI (SEQ ID NO:20), FQILY (SEQ ID NO:21), ILFQY (SEQ ID NO:22), FQIY (SEQ ID NO:23), WWW, WWPWW (SEQ ID NO:24), WPWW (SEQ ID NO:25), WWPW (SEQ ID NO:26) or any combination thereof.

Suitably, the or each hydrophobic domain consists of one of the following sequences FQILY (SEQ ID NO:21), WWW, WWPWW (SEQ ID NO:24).

Suitably, the or each hydrophobic domain consists of FQILY (SEQ ID NO:21).

Suitably each hydrophobic domain in the peptide may have the same sequence or a different sequence.

Peptide Carrier

The present invention relates to conjugates comprising a peptide carrier for use in transporting therapeutic nucleic acids formed of trinucleotide repeats in the treatment of medical conditions.

The peptide has a sequence that is a contiguous single molecule, therefore the domains of the peptide are contiguous. Suitably, the peptide comprises several domains in a linear arrangement between the N-terminus and the C-terminus. Suitably, the domains are selected from cationic domains and hydrophobic domains described above. Suitably, the peptide consists of cationic domains and hydrophobic domains wherein the domains are as defined above.

Each domain has common sequence characteristics as described in the relevant sections above, but the exact sequence of each domain is capable of variation and modification. Thus a range of sequences is possible for each domain. The combination of each possible domain sequence yields a range of peptide structures, each of which form part of the present invention. Features of the peptide structures are described below.

Suitably, a hydrophobic domain separates any two cationic domains. Suitably, each hydrophobic domain is flanked by cationic domains on either side thereof.

Suitably no cationic domain is contiguous with another cationic domain.

In one embodiment, the peptide comprises one hydrophobic domain flanked by two cationic domains in the following arrangement:

[cationic domain]—[hydrophobic domain]—[cationic domain]

Therefore, suitably the hydrophobic domain may be known as the core domain and each of the cationic domains may be known as an arm domain. Suitably, the hydrophobic arm domains flank the cationic core domain on either side thereof.

In one embodiment, the peptide consists of two cationic domains and one hydrophobic domain.

In one embodiment, the peptide consists of one hydrophobic core domain flanked by two cationic arm domains.

In one embodiment, the peptide consists of one hydrophobic core domain comprising a sequence selected from: YQFLI (SEQ ID NO:20), FQILY (SEQ ID NO:21), ILFQY (SEQ ID NO:22), FQIY (SEQ ID NO:23), WWW, WWPWW (SEQ ID NO:24), WPWW (SEQ ID NO:25), and WWPW (SEQ ID NO:26), flanked by two cationic arm domains each comprising a sequence selected from: RBRRBRR (SEQ ID NO:1), RBRBR (SEQ ID NO:2), RBRR (SEQ ID NO:3), RBRRBR (SEQ ID NO:4), RRBRBR (SEQ ID NO:5), RBRRB (SEQ ID NO:6), BRBR (SEQ ID NO:7), RBHBH (SEQ ID NO:8), HBHBR (SEQ ID NO:9), RBRHBHR (SEQ ID NO:10), RBRBBHR (SEQ ID NO:11), RBRRBH (SEQ ID NO:12), HBRRBR (SEQ ID NO:13), HBHBH (SEQ ID NO:14), BHBH (SEQ ID NO:15), BRBSB (SEQ ID NO:16), BRB[Hyp]B (SEQ ID NO:17), R[Hyp]H[Hyp]HB (SEQ ID NO:18), and R[Hyp]RR[Hyp]R (SEQ ID NO:19).

In one embodiment, the peptide consists of one hydrophobic core domain comprising a sequence selected from: FQILY (SEQ ID NO:21), WWW, and WWPWW (SEQ ID NO:24) flanked by two cationic arm domains comprising a sequence selected from: RBRRBRR (SEQ ID NO:1), RBRBR (SEQ ID NO:2), RBRRBR (SEQ ID NO:4), RBRRB (SEQ ID NO:6), BRBR (SEQ ID NO:7), and RBHBH (SEQ ID NO:8).

In one embodiment, the peptide consists of one hydrophobic core domain comprising the sequence: FQILY (SEQ ID NO:21), flanked by two cationic arm domains comprising a sequence selected from: RBRRBRR (SEQ ID NO:1), RBRBR (SEQ ID NO:2), RBRRBR (SEQ ID NO:4), RBRRB (SEQ ID NO:6), BRBR (SEQ ID NO:7), RBHBH (SEQ ID NO:8).

In any such embodiment, further groups may be present such as a linker, terminal modification and/or therapeutic molecule.

Suitably, the peptide is N-terminally modified.

Suitably the peptide is N-acetylated, N-methylated, N-trifluoroacetylated, N-trifluoromethylsulfonylated, or N-methylsulfonylated. Suitably, the peptide is N-acetylated.

Optionally, the N-terminus of the peptide may be unmodified.

In one embodiment, the peptide is N-acetylated.

Suitably, the peptide comprises a C-terminal modification selected from: Carboxy-, Thioacid-, Aminooxy-, Hydrazino-, thioester-, azide, strained alkyne, strained alkene, aldehyde-, thiol or haloacetyl-group.

Advantageously, the C-terminal or N-terminal modification may provide a means for linkage of the peptide to the therapeutic molecule.

Accordingly, the C-terminal modification or the N-terminal modification may comprise the linker and vice versa. Suitably, the C-terminal modification or the N-terminal modification may consist of the linker or vice versa. Suitable linkers are described herein elsewhere.

Suitably, the peptide comprises a C-terminal carboxyl group.

Suitably, the C-terminal carboxyl group is provided by a glycine, beta-alanine, glutamic acid, or gamma-Aminobutyric acid residue.

In one embodiment, the C terminal carboxyl group is provided by a beta-alanine residue.

Suitably, the C terminal residue is a linker. Suitably, the C terminal beta-alanine residue is a linker.

Suitably, therefore each cationic domain may further comprise an N or C terminal modification. Suitably the cationic domain at the C terminus comprises a C-terminal modification. Suitably the cationic domain at the N terminus comprises a N-terminal modification. Suitably, the cationic domain at the C terminus comprises a linker group, suitably, the cationic domain at the C terminus comprises a C-terminal beta-alanine. Suitably, the cationic domain at the N terminus is N-acetylated.

The peptide of the present invention is defined as having a total length of 40 amino acid residues or less. The peptide may therefore be regarded as an oligopeptide.

Suitably, the peptide has a total length of between 3-30 amino acid residues, suitably of between 5-25 amino acid residues, of between 10-25 amino acid residues, of between 13-23 amino acid residues, of between 15-20 amino acid residues.

Suitably, the peptide has a total length of at least 12, at least 13, at least 14, at least 15, at least 16, at least 17 amino acid residues.

Suitably the peptide is capable of penetrating cells. The peptide may therefore be regarded as a cell-penetrating peptide.

Suitably, the peptide is for attachment to a therapeutic molecule. Suitably, the peptide is for transporting a therapeutic molecule into a target cell. Suitably, the peptide is for delivering a therapeutic molecule into a target cell. The peptide is therefore regarded peptide carrier.

Suitably, the peptide carrier is capable of penetrating into cells and tissues, suitably into the nucleus of cells. Suitably into muscle tissues.

Suitably, the peptide carrier may be selected from any of the following sequences:

(SEQ ID NO: 27)
RBRRBRRFQILYRBRBR
(SEQ ID NO: 28)
RBRRBRRFQILYRBRR
(SEQ ID NO: 29)
RBRRBRFQILYRRBRBR
(SEQ ID NO: 30)
RBRBRFQILYRBRRBRR
(SEQ ID NO: 31)
RBRRBRRYQFLIRBRBR
(SEQ ID NO: 32)
RBRRBRRILFQYRBRBR
(SEQ ID NO: 33)
RBRRBRFQILYRBRBR
(SEQ ID NO: 34)
RBRRBFQILYRBRRBR
(SEQ ID NO: 35)
RBRRBRFQILYBRBR
(SEQ ID NO: 36)
RBRRBFQILYRBRBR
(SEQ ID NO: 37)
RBRRBRRFQILYRBHBH
(SEQ ID NO: 38)
RBRRBRRFQILYHBHBR
(SEQ ID NO: 39)
RBRRBRRFQILYHBRBH
(SEQ ID NO: 40)
RBRRBRRYQFLIRBHBH
(SEQ ID NO: 41)
RBRRBRRILFQYRBHBH
(SEQ ID NO: 42)
RBRHBHRFQILYRBRBR
(SEQ ID NO: 43)
RBRBBHRFQILYRBHBH
(SEQ ID NO: 44)
RBRRBRFQILYRBHBH
(SEQ ID NO: 45)
RBRRBRFQILYHBHBH
(SEQ ID NO: 46)
RBRRBHFQILYRBHBH
(SEQ ID NO: 47)
HBRRBRFQILYRBHBH
(SEQ ID NO: 48)
RBRRBFQILYRBHBH
(SEQ ID NO: 49)
RBRRBRFQILYBHBH
(SEQ ID NO: 50)
RBRRBRYQFLIHBHBH
(SEQ ID NO: 51)
RBRRBRILFQYHBHBH
(SEQ ID NO: 52)
RBRRBRRFQILYHBHBH

Suitably, the peptide may be selected from any of the following additional sequences:

(SEQ ID NO: 53)
RBRRBRFQILYBRBS
(SEQ ID NO: 54)
RBRRBRFQILYBRB[Hyp]
(SEQ ID NO: 55)
RBRRBRFQILYBR[Hyp]R
(SEQ ID NO: 56)
RRBRRBRFQILYBRBR
(SEQ ID NO: 57)
BRRBRRFQILYBRBR
(SEQ ID NO: 58)
RBRRBRWWWBRBR
(SEQ ID NO: 59)
RBRRBRWWPWWBRBR
(SEQ ID NO: 60)
RBRRBRWPWWBRBR
(SEQ ID NO: 61)
RBRRBRWWPWBRBR
(SEQ ID NO: 62)
RBRRBRRWWWRBRBR
(SEQ ID NO: 63)
RBRRBRRWWPWWRBRBR
(SEQ ID NO: 64)
RBRRBRRWPWWRBRBR
(SEQ ID NO: 65)
RBRRBRRWWPWRBRBR
(SEQ ID NO: 66)
RBRRBRRFQILYBRBR
(SEQ ID NO: 67)
RBRRBRRFQILYRBR
(SEQ ID NO: 68)
BRBRBWWPWWRBRRBR
(SEQ ID NO: 69)
RBRRBRRFQILYBHBH
(SEQ ID NO: 70)
RBRRBRRFQIYRBHBH
(SEQ ID NO: 71)
RBRRBRFQILYBRBH
(SEQ ID NO: 72)
RBRRBRFQILYR[Hyp]H[Hyp]H
(SEQ ID NO: 73)
R[Hyp]RR[Hyp]RFQILYRBHBH
(SEQ ID NO: 74)
R[Hyp]RR[Hyp]RFQILYR[Hyp]H[Hyp]H
(SEQ ID NO: 75)
RBRRBRWWWRBHBH
(SEQ ID NO: 76)
RBRRBRWWPRBHBH
(SEQ ID NO: 77)
RBRRBRPWWRBHBH
(SEQ ID NO: 78)
RBRRBRWWPWWRBHBH
(SEQ ID NO: 79)
RBRRBRWWPWRBHBH
(SEQ ID NO: 80)
RBRRBRWPWWRBHBH
(SEQ ID NO: 81)
RBRRBRRWWWRBHBH
(SEQ ID NO: 82)
RBRRBRRWWPWWRBHBH
(SEQ ID NO: 83)
RBRRBRRWPWWRBHBH
(SEQ ID NO: 84)
RBRRBRRWWPWRBHBH
(SEQ ID NO: 85)
RRBRRBRFQILYRBHBH
(SEQ ID NO: 86)
BRRBRRFQILYRBHBH
(SEQ ID NO: 87)
RRBRRBRFQILYBHBH
(SEQ ID NO: 88)
BRRBRRFQILYBHBH
(SEQ ID NO: 89)
RBRRBHRFQILYRBHBH
(SEQ ID NO: 90)
RBRRBRFQILY[Hyp]R[Hyp]R
(SEQ ID NO: 91)
R[Hyp]RR[Hyp]RFQILYBRBR
(SEQ ID NO: 92)
R[Hyp]RR[Hyp]RFQILY[Hyp]R[Hyp]R
(SEQ ID NO: 93)
RBRRBRWWWBRBR
(SEQ ID NO: 94)
RBRRBRWWPWWBRBR

Suitably the peptide consists of one of the following sequences:

(SEQ ID NO: 27)
RBRRBRRFQILYRBRBR
(SEQ ID NO: 31)
RBRRBRRYQFLIRBRBR
(SEQ ID NO: 32)
RBRRBRRILFQYRBRBR
(SEQ ID NO: 35)
RBRRBRFQILYBRBR
(SEQ ID NO: 37)
RBRRBRRFQILYRBHBH
(SEQ ID NO: 38)
RBRRBRRFQILYHBHBR
(SEQ ID NO: 44)
RBRRBRFQILYRBHBH
In one embodiment, the peptide consists of the
following sequence: RBRRBRFQILYBRBR
(SEQ ID NO: 35).
In one embodiment, the peptide consists of the
following sequence: RBRRBRRFQILYRBHBH
(SEQ ID NO: 37).
In one embodiment, the peptide consists of the
following sequence: RBRRBRFQILYRBHBH
(SEQ ID NO: 44).

Therapeutic Molecule

The peptide carrier is covalently linked to a therapeutic molecule in order to provide a conjugate of the invention, wherein the therapeutic molecule is a nucleic acid comprising a plurality trinucleotide repeats.

Suitably the nucleic acid may be selected from: an antisense oligonucleotide (such as PNA, PMO), mRNA, gRNA (for example in the use of CRISPR/Cas9 technology), short interfering RNA, micro RNA, and antagomiRNA.

Suitably, the nucleic acid is an antisense oligonucleotide.

Suitably, the antisense oligonucleotide is a phosphorodiamidate morpholino oligonucleotide (PMO).

Alternatively the antisense oligonucleotide may be a modified PMO or any other charge-neutral antisense oligonucleotide such as a peptide nucleic acid (PNA), a chemically modified PNA such as a gamma-PNA (Bahal, Nat.Comm. 2016), oligonucleotide phosphoramidate (where the non-bridging oxygen of the phosphate is substituted by an amine or alkylamine such as those described in WO2016028187A1, or any other partially or fully charge-neutralized oligonucleotide.

Suitably, the nucleic acid consists of a plurality of trinucleotide repeats.

Suitably the nucleic acid comprises any trinucleotide repeat. Suitably the nucleic acid comprises trinucleotide repeats selected from: GTC, CAG, GCC, GGC, CTT, and CCG repeats. Suitably the nucleic acid consists of trinucleotide repeats selected from: GTC, CAG, GCC, GGC, CTT, and CCG repeats.

Suitably the nucleic acid comprises CAG repeats. Suitably the nucleic acid consists of CAG repeats.

In one embodiment, the nucleic acid is an antisense oligonucleotide comprising CAG repeats. In one embodiment, the nucleic acid is an antisense oligonucleotide consisting of CAG repeats.

Suitably the nucleic acid comprises, or consists of, a plurality of trinucleotide repeats. Suitably the nucleic acid comprises, or consists of at least 2 trinucleotide repeats. Suitably the nucleic acid comprises, or consists of, between 5-50 trinucleotide repeats. Suitably the nucleic acid comprises, or consists of, between 5-40 trinucleotide repeats. Suitably the nucleic acid comprises, or consists of, between 5-30 trinucleotide repeats. Suitably the nucleic acid comprises, or consists of, between 5-20 trinucleotide repeats. Suitably the nucleic acid comprises, or consists of, between 5-10 trinucleotide repeats. Suitably the nucleic acid comprises, or consists of, 7 trinucleotide repeats.

In one embodiment, the nucleic acid is an antisense oligonucleotide comprising 7 CAG repeats. In one embodiment, the nucleic acid is an antisense oligonucleotide consisting of 7 CAG repeats. Suitably, in such an embodiment, the nucleic acid is an antisense oligonucleotide consisting of [CAG]₇.

Suitably the nucleic acid is complementary to a microsatellite region, suitably to a repeat expansion, suitably to a trinucleotide repeat expansion.

Suitably, the nucleic acid targets and binds to microsatellite regions. Suitably the microsatellite regions comprise repeat expansions, suitably they comprise trinucleotide repeat expansions.

In some embodiments, the repeat expansions may comprise higher repeat expansions, such as tetra, penta, hexa, hepta, octo, nona, or deca, etc. repeat expansions comprising four, five, six, seven, eight, nine or ten nucleotides per repeat respectively.

Therefore, in some embodiments, the therapeutic molecule is a nucleic acid comprising a plurality of tetra, penta, hexa, hepta, octo, nona, or deca nucleotide repeats. Therefore, in some embodiments, the therapeutic molecule is a nucleic acid consisting of a plurality of tetra, penta, hexa, hepta, octo, nona, or deca nucleotide repeats.

Any of the statements herein relating to nucleic acids comprising trinucleotide repeats apply equally to nucleic acids comprising higher nucleotide repeats.

Suitably, the nucleic acid binds to a complementary microsatellite region, suitably to a complementary region of repeat expansion, suitably to a complementary region of trinucleotide repeat expansion.

Suitably the microsatellite regions are present in DNA or RNA. Suitably the microsatellite regions are present in RNA.

Suitably the microsatellite regions may be present in coding or non-coding sequences. Suitably the microsatellite regions are present in non-coding sequences such as the 3′ or 5′ UTRs. Suitably the microsatellite regions are present in the 3′ UTR.

Suitably, the nucleic acid may be formed of a trinucleotide repeat that binds to a complementary trinucleotide repeat expansion.

Suitably, the nucleic acid may be formed of a trinucleotide repeat that binds to a complementary trinucleotide repeat expansion in RNA.

Suitably, the nucleic acid may be formed of a trinucleotide repeat that binds to a complementary trinucleotide repeat expansion in a non-coding sequence of RNA.

Suitably, the nucleic acid may be formed of a trinucleotide repeat that binds to a complementary trinucleotide repeat expansion in an untranslated region of RNA.

In one embodiment, the nucleic acid may be formed of a trinucleotide repeat that binds to a complementary trinucleotide repeat expansion in the 3′UTR of RNA.

Optionally, lysine residues may be added to one or both ends of the nucleic acid (such as a PMO or PNA) before attachment to the peptide carrier to improve water solubility.

Trinucleotide Repeat Disorder

The conjugate of the present invention is for use as a medicament, preferably in the prevention or treatment of trinucleotide repeat disorders.

Suitably a trinucleotide repeat disorder is a genetic disorder caused by a trinucleotide repeat expansion, which may otherwise be known as a triplet repeat expansion.

Suitably the trinucleotide repeat expansion is present in a gene. Suitably the trinucleotide repeat expansion is present in a gene selected from: ATN1, HTT, AR, ATXN1, ATXN2, ATXN3, CACNA1A, ATXN7, TBP, FMR1, AFF2, FXN, DMPK, SCA8, JPH3, and PPP2R2B.

Suitably the trinucleotide repeat expansion is present in the AR, SCA8 or DMPK gene.

In one embodiment, the trinucleotide repeat expansion is present in the DMPK gene.

Suitably the trinucleotide repeat expansion is formed of repeats selected from: CAG, CTG, CGG, CCG, GAA , TTC and GGC.

Suitably the trinucleotide repeat expansion is formed of CAG or CTG repeats.

In one embodiment, the trinucleotide repeat expansion is formed of CTG repeats.

Typically trinucleotide repeat disorders result from the presence of a particular trinucleotide repeat expansion found in a particular gene. Typically the number of trinucleotide repeats that are present in the gene is higher than the number of trinucleotide repeats present in the same gene in a normal healthy subject.

Suitably, the trinucleotide repeat expansion is a CAG repeat in a gene selected from: ATN1, HTT, AR, ATXN1, ATXN, ATXN3, CACNA1A, ATXN7, JPH3, and TBP.

Suitably trinucleotide repeat disorders resulting from CAG repeats are termed ‘polyglutamine diseases’. Suitably therefore, the trinucleotide repeat disorder may be a polyglutamine disorder. Suitably the polyglutamine disorder may be selected from: DRPLA (Dentatorubropallidoluysian atrophy), HD (Huntingdon's disease), HDL2 (Huntingdon disease like syndrome 2), SBMA (spinal and bulbar muscular atrophy), SCA1 (spinocerebellar ataxia type 1), SCA2 (spinocerebellar ataxia type 2), SCA3 (spinocerebellar ataxia type 3 or Machado-Jospeh disease), SCA6 (spinocerebellar ataxia type 6), SCA7 (spinocerebellar ataxia type 7), and SCA17 (spinocerebellar ataxia type 17).

Suitably the trinucleotide repeat expansion is a CGG repeat in a gene selected from: FMR1.

Suitably the trinucleotide repeat expansion is a CCG repeat in a gene selected from: AFF2.

Suitably the trinucleotide repeat expansion is a GAA repeat in a gene selected from FXN.

Suitably the trinucleotide repeat expansion is a CTG repeat in a gene selected from DMPK, and ATXN8.

Suitably the trinucleotide repeat expansion is a GTC repeat in a gene selected from JPH3.

Suitably trinucleotide repeat disorders resulting from trinucleotide repeats other than CAG repeats are termed ‘non-polyglutamine diseases’. Suitably therefore, the trinucleotide repeat disorder may be a non-polyglutamine disorder. Suitably the non-polyglutamine disorder may be selected from: HDL2 (Huntingdon disease like syndrome 2), FRAXA (Fragile X syndrome), FXTAS (Fragile X temor/ataxia syndrome), FRAXE (Fragile XE mental retardation), FRDA (Friedrich's ataxia), DM1 (Myotonic dystrophy type 1), SCA8 (spinocerebellar ataxia type 8), and SCA12 (spinocerebellar ataxia type 12).

Suitably the trinucleotide repeat disorder results from an increase in the number of trinucleotide repeats compared to a healthy subject. Suitably, an increase in the number of trinucleotide repeats in a gene compared to the same gene in healthy subject. Suitably the number of trinucleotide repeats in the trinucleotide repeat expansion is increased compared to the number of trinucleotide repeats in a normal healthy subject.

Suitably the number of repeats in the trinucleotide repeat expansion is at least 1.5× the number of repeats in a normal healthy subject. Suitably the number of repeats in the trinucleotide repeat expansion is at least 2×, 3×, 4×, 5, 6×, 7×, 8×, 9×, 10×, 15×, 20×, 25×, 30×, 35×, 40×, 45×, or 50× the number of repeats in a normal healthy subject.

Suitably the trinucleotide repeat disorder results from an increase in the number of repeats in a trinucleotide repeat expansion of at least 1.5× the number of repeats in a normal healthy subject.

Suitably the trinucleotide repeat disorder results from an increase in the number of repeats in a trinucleotide repeat expansion of at least 2×, 3×, 4×, 5, 6×, 7×, 8×, 9×, 10×, 15×, 20×, 25×, 30×, 35×, 40×, 45×, or 50× the number of repeats in a normal healthy subject.

Suitably the number of repeats in the trinucleotide repeat expansion is between 1.5× to 15× the number of repeats in a normal healthy subject.

Suitably the trinucleotide repeat disorder results from a trinucleotide repeat expansion comprising between 1.5× to 15× the number of repeats present in a normal healthy subject.

Suitably, the number of repeats in the trinucleotide expansion is more than 50, more than 75, more than 100, more than 125, more than 150, more than 175, more than 200, more than 225, more than 250.

Suitably the trinucleotide repeat disorder results from a trinucleotide repeat expansion comprising more than 50, more than 75, more than 100, more than 125, more than 150, more than 175, more than 200, more than 225, more than 250 repeats.

Suitably, the number of repeats in the trinucleotide expansion is more than 50.

Suitably the trinucleotide repeat disorder results from a trinucleotide repeat expansion comprising more than 50 repeats.

Suitably, the number of repeats in the trinucleotide expansion is between 50 and 250.

Suitably the trinucleotide repeat disorder results from a trinucleotide repeat expansion comprising between 50 and 250 repeats.

Suitably, the trinucleotide repeat disorder is a non-polyglutamine disorder.

Suitably, the trinucleotide repeat disorder is DM1 or SCA8.

In one embodiment, the trinucleotide repeat disorder is DM1.

In one embodiment, when the trinucleotide repeat disorder is DM1, the number of repeats in the trinucleotide expansion is more than 50. In one embodiment, when the trinucleotide repeat disorder is DM1, the number of CTG repeats in the trinucleotide expansion is more than 50.

In one embodiment, when the trinucleotide repeat disorder is DM1, the number of CTG repeats in the trinucleotide expansion of the DMPK gene is more than 50.

In one embodiment, when the trinucleotide repeat disorder is SCA8, the number of repeats in the trinucleotide expansion is between 110 and 250. In one embodiment, when the trinucleotide repeat disorder is SCA8, the number of CTG repeats in the trinucleotide expansion is between 110 and 250. In one embodiment, when the trinucleotide repeat disorder is SCA8, the number of CTG repeats in the trinucleotide expansion of the ATXN8 is between 110 and 250.

In some embodiments, the conjugate of the present invention is for use as a medicament, preferably in the prevention or treatment of nucleotide repeat disorders.

Suitably a nucleotide repeat disorder is a genetic disorder caused by a nucleotide repeat expansion, which may otherwise be known as a repeat expansion or microsatellite repeat expansion.

Suitably the nucleotide repeat disorder may be caused by a repeat expansion of four, five, six, seven, eight, nine or ten nucleotides.

Suitably the nucleotide repeat expansion may be a higher repeat expansion as discussed hereinabove, such as a tetra, penta, hexa, hepta, octa, nona, or deca nucleotide repeat expansion.

Suitably therefore, the conjugate of the present invention is for use as a medicament, preferably in the prevention or treatment of tetra, penta, hexa, hepta, octa, nona, or deca nucleotide repeat disorders.

Suitably the nucleotide repeat expansion is a tetranucleotide repeat, suitably the tetranucleotide repeat is a CCTG repeat

Suitably therefore, the conjugate of the present invention is for use as a medicament, preferably in the prevention or treatment of DM2 (Myotonic Dystrophy type 2).

Suitably the nucleotide repeat expansion is a pentanucleotide repeat, suitably the pentanucleotide repeat is a ATTCT repeat

Suitably therefore, the conjugate of the present invention is for use as a medicament, preferably in the prevention or treatment of SCA10 (Spinocerebellar Ataxia Type 10).

Suitably therefore, the conjugate of the present invention is for use as a medicament, preferably in the prevention or treatment of SCA31 (Spinocerebellar Ataxia Type 31).

Suitably the nucleotide repeat expansion is a hexanucleotide repeat, suitably the hexanucleotide repeat is a GGCCTG repeat or GGGGCC repeat.

Suitably therefore, the conjugate of the present invention is for use as a medicament, preferably in the prevention or treatment of SCA36 (Spinocerebellar Ataxia Type 36).

Suitably therefore, the conjugate of the present invention is for use as a medicament, preferably in the prevention or treatment of C9ORF72-ALS (Amyotrophic lateral sclerosis).

Any of the statements herein relating to treatment of trinucleotide repeat disorders apply equally to treatment of higher nucleotide repeat disorders, such as tetra, penta, hexa, hepta, octa, nona, or deca nucleotide repeat disorders.

Covalent Link

The peptide carrier present in the conjugate of the invention is covalently linked to the therapeutic molecule.

Suitably, the peptide carrier is covalently linked to the therapeutic molecule at the C-terminus or N-terminus. Suitably, the peptide carrier is covalently linked to the therapeutic molecule at the C-terminus

Suitably, the peptide carrier is covalently linked to the therapeutic molecule through a linker if required. The linker may act as a spacer to separate the peptide sequence from the therapeutic molecule.

The linker may be selected from any suitable sequence.

Suitably the linker is present between the peptide and the therapeutic molecule. Suitably the linker is a separate group to the peptide and the therapeutic molecule. Accordingly, the linker may comprise artificial amino acids.

In one embodiment, the conjugate comprises the peptide carrier covalently linked via a linker to a therapeutic molecule.

In one embodiment, the conjugate comprises the following structure:

[peptide]—[linker]—[therapeutic molecule]

In one embodiment, the conjugate consists of the following structure:

[peptide]—[linker]—[therapeutic molecule]

Suitably any of the peptides listed herein may be used in a conjugate according to the invention. In one embodiment, the conjugate comprises a peptide carrier selected from one of the following sequences: RBRRBRFQILYBRBR (SEQ ID NO:35), RBRRBRRFQILYRBHBH (SEQ ID NO:37) and RBRRBRFQILYRBHBH (SEQ ID NO:44).

Suitably, in any case, the peptide carrier may further comprise N-terminal modifications as described above.

Suitable linkers include, for example, a C-terminal cysteine residue that permits formation of a disulphide, thioether or thiol-maleimide linkage, a C-terminal aldehyde to form an oxime, a click reaction or formation of a morpholino linkage with a basic amino acid on the peptide or a carboxylic acid moiety on the peptide covalently conjugated to an amino group to form a carboxamide linkage.

Suitably, the linker is between 1-5 amino acids in length. Suitably the linker may comprise any linker that is known in the art.

Suitably the linker is selected from any of the following sequences: G, BC, XC, C, GGC, BBC, BXC, XBC, X, XX, B, BB, BX, XB, succinic acid, GABA and E. Suitably, wherein X is 6-aminohexanoic acid.

Suitably the linker may be a polymer, such as for example PEG.

Suitably, the linker is selected from: beta-alanine (B), succinic acid (Succ), GABA (Ab), and glutamic acid (E).

In one embodiment, the linker is beta-alanine (B).

In one embodiment, the peptide carrier is conjugated to the therapeutic molecule through a carboxamide linkage.

The linker of the conjugate may form part of the therapeutic molecule to which the peptide is attached. Alternatively, the attachment of the therapeutic molecule may be directly linked to the C-terminus or N-terminus of the peptide carrier. Suitably, in such embodiments, no linker is required.

Alternatively, the peptide carrier may be chemically conjugated to the therapeutic molecule. Chemical linkage may be via a disulphide, alkenyl, alkynyl, aryl, ether, thioether, triazole, amide, carboxamide, urea, thiourea, semicarbazide, carbazide, hydrazine, oxime, phosphate, phosphoramidate, thiophosphate, boranophosphate, iminophosphates, or thiol-maleimide linkage, for example.

Optionally, cysteine may be added at the N-terminus of a therapeutic molecule to allow for disulphide bond formation to the peptide carrier, or the N-terminus may undergo bromoacetylation for thioether conjugation to the peptide carrier.

In one embodiment, the conjugate comprises a peptide carrier selected from one of the following sequences: RBRRBRFQILYBRBR (SEQ ID NO:35), RBRRBRRFQILYRBHBH (SEQ ID NO:37) and RBRRBRFQILYRBHBH (SEQ ID NO:44) covalently linked by a linker to an antisense oligonucleotide comprising CAG repeats, wherein the linker is selected from: beta-alanine (B), GABA (Ab), and glutamic acid (E).

In one embodiment, the conjugate comprises a peptide carrier selected from one of the following sequences: RBRRBRFQILYBRBR (SEQ ID NO:35), RBRRBRRFQILYRBHBH (SEQ ID NO:37) and RBRRBRFQILYRBHBH (SEQ ID NO:44) covalently linked by a linker to an antisense oligonucleotide consisting of CAG repeats, wherein the linker is selected from: beta-alanine (B), GABA (Ab), and glutamic acid (E).

In one embodiment, the conjugate comprises a peptide carrier selected from one of the following sequences: RBRRBRFQILYBRBR (SEQ ID NO:35), RBRRBRRFQILYRBHBH (SEQ ID NO:37) and RBRRBRFQILYRBHBH (SEQ ID NO:44) covalently linked by a linker to an antisense oligonucleotide consisting of seven CAG repeats, wherein the linker is selected from: beta-alanine (B), GABA (Ab), and glutamic acid (E).

In one embodiment, the conjugate comprises peptide carrier RBRRBRFQILYBRBR (SEQ ID NO:35) covalently linked by a beta-alanine (B) to an antisense oligonucleotide consisting of seven CAG repeats. (DPEP1.9)

In one embodiment, the conjugate comprises peptide carrier RBRRBRFQILYBRBR (SEQ ID NO:35) covalently linked by a glutamic acid (E) to an antisense oligonucleotide consisting of seven CAG repeats. (DPEP1.9b) In one embodiment, this conjugate has increased penetration into diaphragm tissue. Suitably increased penetration into diaphragm is useful for treating muscular disorders which affect the respiratory system such as myotonic dystrophy.

In one embodiment, the conjugate comprises peptide carrier RBRRBRRFQILYRBHBH (SEQ ID NO:37) covalently linked by a beta-alanine (B) to an antisense oligonucleotide consisting of seven CAG repeats. (DPEP3.1) In one embodiment, this conjugate has increased penetration into muscular tissue. Suitably increased penetration into muscle is useful for treating muscular disorders.

In one embodiment, the conjugate comprises peptide carrier RBRRBRRFQILYRBHBH (SEQ ID NO:37) covalently linked by a glutamic acid (E) to an antisense oligonucleotide consisting of seven CAG repeats. (DPEP3.1b) In one embodiment, this conjugate has increased penetration into muscular tissue. Suitably increased penetration into muscle is useful for treating muscular disorders.

In one embodiment, the conjugate comprises peptide carrier RBRRBRRFQILYRBHBH (SEQ ID NO:37) covalently linked by a GABA (Ab) to an antisense oligonucleotide consisting of seven CAG repeats. (DPEP3.1a)

In one embodiment, the conjugate comprises peptide carrier RBRRBRFQILYRBHBH (SEQ ID NO:44) covalently linked by a beta-alanine (B) to an antisense oligonucleotide consisting of seven CAG repeats. (DPEP3.8) In one embodiment, this conjugate has increased penetration into muscular tissue. Suitably increased penetration into muscle is useful for treating muscular disorders.

In one embodiment, the conjugate comprises peptide carrier RBRRBRFQILYRBHBH (SEQ ID NO:44) covalently linked by a glutamic acid (E) to an antisense oligonucleotide consisting of seven CAG repeats. (DPEP.3.8b) In one embodiment, this conjugate has increased penetration into diaphragm tissue. Suitably increased penetration into diaphragm is useful for treating muscular disorders which affect the respiratory system such as myotonic dystrophy.

Any of the above conjugates may be acetylated at the N-terminus.

Pharmaceutical Composition and Administration The conjugate of the invention may formulated into a pharmaceutical composition as noted above.

According to a sixth aspect of the present invention, the pharmaceutical composition comprises a conjugate of the invention.

Suitably, the pharmaceutical composition may further comprise one or more pharmaceutically acceptable components such as one or more diluents, adjuvants or carriers.

Suitable pharmaceutically acceptable diluents, adjuvants and carriers are well known in the art.

As used herein, the phrase “pharmaceutically acceptable” refers to those ligands, materials, formulations, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier”, as used herein, refers to a pharmaceutically acceptable material, formulation or vehicle, such as a liquid or solid filler, diluent, excipient, solvent or encapsulating material, involved in carrying or transporting the conjugate from one organ or portion of the body, to another organ or portion of the body. Each peptide must be “acceptable” in the sense of being compatible with the other components of the composition e.g. the peptide and therapeutic molecule, and not injurious to the individual.

Lyophilized compositions, which may be reconstituted and administered, are also within the scope of the present composition.

Pharmaceutically acceptable carriers may be, for example, excipients, vehicles, diluents, and combinations thereof. For example, where the compositions are to be administered orally, they may be formulated as tablets, capsules, granules, powders, or syrups; or for parenteral administration, they may be formulated as injections, drop infusion preparations, or suppositories. These compositions can be prepared by conventional means, and, if desired, the active compound (i.e. conjugate) may be mixed with any conventional additive, such as an excipient, a binder, a disintegrating agent, a lubricant, a corrigent, a solubilizing agent, a suspension aid, an emulsifying agent, a coating agent, or combinations thereof.

It should be understood that the pharmaceutical compositions of the present disclosure can further include additional known therapeutic agents, drugs, modifications of compounds into prodrugs, and the like for alleviating, mediating, preventing, and treating the diseases, disorders, and conditions described herein under medical use.

Suitably, the pharmaceutical composition is for use as a medicament. Suitably for use as a medicament in the same manner as described herein for the conjugate. All features described herein in relation to medical treatment using the conjugate apply to the pharmaceutical composition.

Accordingly, in a further aspect of the invention there is provided a pharmaceutical composition according to the sixth aspect for use as a medicament. In a further aspect, there is provided a method of preventing or treating a subject for a disease condition comprising administering an effective amount of a pharmaceutical composition according to the sixth aspect to the subject.

Suitably, wherein the pharmaceutical composition is for use in the prevention or treatment of a trinucleotide disorder, and suitably wherein the method of prevention or treatment is of a trinucleotide disorder in a subject.

Prevention or Treatment

The conjugate of the invention may be used as a medicament for the prevention or treatment of a disease, preferably a trinucleotide repeat disorder.

The medicament may be in the form of a pharmaceutical composition as defined above.

A method of prevention or treatment of a subject in need of treatment for a disease condition is also provided, the method comprising the step of administering a therapeutically effective amount of the conjugate to the subject.

Suitably, the conjugate is for use in the prevention or treatment of trinucleotide repeat disorders.

Suitable genes comprising trinucleotide repeat expansions and details of the trinucleotide repeat disorders resulting therefrom are detailed hereinabove.

Alternatively, the conjugate may be for use in the prevention or treatment of other nucleotide repeat disorders. Suitable details of such higher repeat expansions and resulting nucleotide repeat disorders are detailed above.

Specific mechanisms of how the nucleic acid formed of trinucleotide repeats may act to treat a trinucleotide repeat disorder will be different depending on the trinucleotide repeat disorder in question. Suitably the nucleic acid binds to the trinucleotide repeat expansion, in the gene or in the transcript. Suitably the nucleic acid reduces the level of transcripts comprising a trinucleotide repeat expansion. Suitably the nucleic acid prevents the pathological effects of the trinucleotide repeat expansion, and hence the trinucleotide repeat disorder. The same applies to other nucleotide repeat disorders.

Suitably, therefore the conjugate improves the physiological condition of subjects.

For example, the therapeutic nucleic acid of the conjugate may be operable to correct splicing defects resulting from a trinucleotide repeat disorder. Suitably the therapeutic nucleic acid of the conjugate may be operable to normalise splicing in a subject with a trinucleotide repeat disorder.

Suitably, the therapeutic nucleic acid of the conjugate is operable to bind a transcript of the DMPK gene. Suitably, the therapeutic nucleic acid of the conjugate is operable to bind repeat expansions present in a transcript of the DMPK gene. Suitably, the therapeutic nucleic acid of the conjugate is operable to bind CUG repeat expansions present in a transcript of the DMPK gene.

Suitably, therefore the conjugate reduces the levels of DMPK transcripts. Suitably, therefore the conjugate reduces the levels of DMPK transcripts having repeat expansions. Suitably, therefore the conjugate reduces the levels of DMPK transcripts having CUG repeat expansions.

Suitably, therefore the conjugate reduces the number nuclear foci. Suitably the conjugate prevents nuclear foci interacting with the splicing machinery of a cell. Suitably the conjugate prevents nuclear foci interacting with MBNL1. Suitably the conjugate prevents nuclear foci sequestering MBNL1.

Suitably these effects are for use in the prevention or treatment of DM1.

Suitably the conjugate decreases myotonia in a subject with DM1 by 10%, 15%, 20%, 25%, 30%, 35%, 40%, 45%, 50%, 55%, 60%, 65%, 70%, 80%, 90%, 100% when compared to healthy subjects. Suitably the conjugate decreases myotonia in a subject with DM1 by at least 50%. Suitably the conjugate decreases myotonia in a subject with DM1 by between 50-100%

Suitably the conjugate reduces nuclear foci in myoblasts in a subject with DM1 by 10%, 15%, 20%, 25%, 30%, 35%, 40%, 45%, 50%, 55%, 60%, 65%, 70%, 80%, 90%. Suitably the conjugate reduces nuclear foci in myoblasts in a subject with DM1 by at least 50%. Suitably the conjugate reduces nuclear foci in myoblasts in a subject with DM1 by between 50-90%.

Suitably the conjugate corrects cardiac conduction in a subject with DM1 by 10%, 15%, 20%, 25%, 30%, 35%, 40%, 45%, 50%. Suitably the conjugate improves cardiac conduction in a subject with DM1 by at least 10%. Suitably the conjugate improves cardiac conductivity in a subject with DM1 by between 10-50%.

Suitably the conjugate improves motor function in a subject with DM1 by 10%, 15%, 20%, 25%, 30%, 35%, 40%, 45%, 50%. Suitably the conjugate improves motor function in a subject with DM1 by at least 10%. %. Suitably the conjugate improves motor function in a subject with DM1 by between 10-50%.

Suitably the conjugate improves muscle force relative to weight in a subject with DM1 by 10%, 15%, 20%, 25%, 30%, 35%, 40%, 45%, 50%. Suitably the conjugate improves muscle force relative to weight in a subject with DM1 by at least 10%. Suitably the conjugate improves muscle force relative to weight in a subject with DM1 by between 10-50%.

Suitably, the subject to be treated may be any animal or human. Suitably, the subject may be a non-human mammal. Suitably the subject may be male or female.

Suitably, the subject to be treated may be any age. Suitably the subject to be treated is aged between 0-40 years, suitably 0-30, suitably 0-25, suitably 0-20 years of age.

Suitably, the conjugate is for administration to a subject systemically for example by intramedullary, intrathecal, intraventricular, intravitreal, enteral, parenteral, intravenous, intra-arterial, intramuscular, intratumoral, subcutaneous oral or nasal routes.

In one embodiment, the conjugate is for administration to a subject intravenously.

In one embodiment, the conjugate is for administration to a subject intravenously by injection.

Suitably, the conjugate is for administration to a subject in a “therapeutically effective amount”, by which it is meant that the amount is sufficient to show benefit to the individual. The actual amount administered, and rate and time-course of administration, will depend on the nature and severity of the disease being treated. Decisions on dosage are within the responsibility of general practitioners and other medical doctors. Examples of the techniques and protocols can be found in Remington's Pharmaceutical Sciences, 20th Edition, 2000, pub. Lippincott, Williams & Wilkins.

Exemplary doses may be between 0.01 mg/kg and 50 mg/kg, 0.05 mg/kg and 40 mg/kg, 0.1 mg/kg and 30 mg/kg, 0.5 mg/kg and 18 mg/kg, 1 mg/kg and 16 mg/kg, 2mg/kg and 15 mg/kg, 5 mg/kg and 10 mg/kg, 10 mg/kg and 20 mg/kg, 12 mg/kg and 18 mg/kg, 13 mg/kg and 17 mg/kg.

Advantageously, the dosage of the conjugates of the present invention is an order or magnitude lower than the dosage required to see any effect from the therapeutic nucleic acid alone.

Suitably, after administration of the conjugate of the present invention, one or more markers of toxicity are significantly reduced compared to conjugates using currently available peptide carriers.

Suitable markers of toxicity may be markers of nephrotoxicity.

Suitable markers of toxicity include serum KIM-1, NGAL, BUN, creatinine, alkaline phosphatase, alanine transferase, and aspartate aminotransferase levels.

Suitable further markers of toxicity include urine sodium, potassium, chloride, urea, creatinine, calcium, phosphorous, glucose, uric acid, magnesium and protein levels.

Suitably the level of at least one of KIM-1, NGAL, and BUN is reduced after administration of the conjugate of the present invention when compared to conjugates using currently available peptide carriers.

Suitably the levels of each of KIM-1, NGAL, and BUN are reduced after administration of the conjugates of the present invention when compared to conjugates using currently available peptide carriers.

Suitably, the levels of the or each marker/s is significantly reduced when compared to conjugates using currently available peptide carriers.

Suitably the levels of the or each marker/s is reduced by up to 5%, 10%, 15%, 20%, 25%, 30%, 35%, 40%, 45%, 50% after administration of the conjugates of the present invention when compared to conjugates using currently available peptide carriers.

Advantageously, the toxicity of the conjugates is significantly reduced compared to prior peptides and conjugates. In particular, KIM-1 and NGAL-1 are markers of toxicity and these are significantly reduced by up to 120 times compared to conjugates using currently available peptide carriers.

Suitably, the long term toxicity of the conjugate is negligible. Suitably there are no long term toxic effects of the conjugate.

Suitably the conjugate has no significant effects on gene expression in the subject, beyond the intended effect on the target trinucleotide repeat expansion. Suitably the conjugate has no negative effects on gene expression in the subject.

Suitably, after administration of the conjugate of the present invention, cell viability is significantly improved compared to conjugates using currently available peptide carriers.

Suitably, after administration of the conjugate of the present invention, myoblast and hepatocyte viability is significantly improved compared to conjugates using currently available peptide carriers. Suitably, after administration of the conjugate of the present invention, myoblast and hepatocyte viability is increased by up to 5%, 10%, 15%, 20%, 25%, 30%, 35%, 40%, 45%, 50% compared to conjugates using currently available peptide carriers.

Suitably, after administration of the conjugate of the present invention, cell viability is significantly improved compared to conjugates using currently available peptide carriers.

Suitably, after administration of the conjugate of the present invention, recovery time is decreased by up to 5%, 10%, 15%, 20%, 25%, 30%, 35%, 40%, 45%, 50% compared to conjugates using currently available peptide carriers.

Suitably, after administration of the conjugate of the present invention, recovery time is less than 60 minutes, less than 50 minutes, less than 40 minutes, less than 30 minutes, less than 20 minutes, less than 10 minutes, or less than 5 minutes. Suitably, after administration of the conjugate of the present invention, there is no recovery time.

Nucleic Acids and Hosts

Peptide carriers of the invention may be produced by any standard protein synthesis method, for example chemical synthesis, semi-chemical synthesis or through the use of expression systems.

Accordingly, the present invention also relates to the nucleotide sequences comprising or consisting of the DNA coding for the conjugates, expression systems e.g. vectors comprising said sequences accompanied by the necessary sequences for expression and control of expression, and host cells and host organisms transformed by said expression systems.

Accordingly, a nucleic acid encoding a conjugate according to the present invention is also provided.

Suitably, the nucleic acids may be provided in isolated or purified form.

An expression vector comprising a nucleic acid encoding a conjugate according to the present invention is also provided.

Suitably, the vector is a plasmid.

Suitably the vector comprises a regulatory sequence, e.g. promoter, operably linked to a nucleic acid encoding a conjugate according to the present invention. Suitably, the expression vector is capable of expressing the conjugate when transfected into a suitable cell, e.g. mammalian, bacterial or fungal cell.

A host cell comprising the expression vector of the invention is also provided.

Expression vectors may be selected depending on the host cell into which the nucleic acids of the invention may be inserted. Such transformation of the host cell involves conventional techniques such as those taught in Sambrook et al [Sambrook, J., Russell, D. (2001) Molecular Cloning: A Laboratory Manual, Cold Spring Harbor Laboratory Press, NY, USA]. Selection of suitable vectors is within the skills of the person knowledgeable in the field. Suitable vectors include plasmids, bacteriophages, cosmids, and viruses.

The conjugates produced may be isolated and purified from the host cell by any suitable method e.g. precipitation or chromatographic separation e.g. affinity chromatography.

Suitable vectors, hosts and recombinant techniques are well known in the art.

In this specification the term “operably linked” may include the situation where a selected nucleotide sequence and regulatory nucleotide sequence are covalently linked in such a way as to place the expression of a nucleotide coding sequence under the control of the regulatory sequence, as such, the regulatory sequence is capable of effecting transcription of a nucleotide coding sequence which forms part or all of the selected nucleotide sequence. Where appropriate, the resulting transcript may then be translated into a desired conjugate.

The invention will now be described with reference to the accompanying figures and examples, in which:

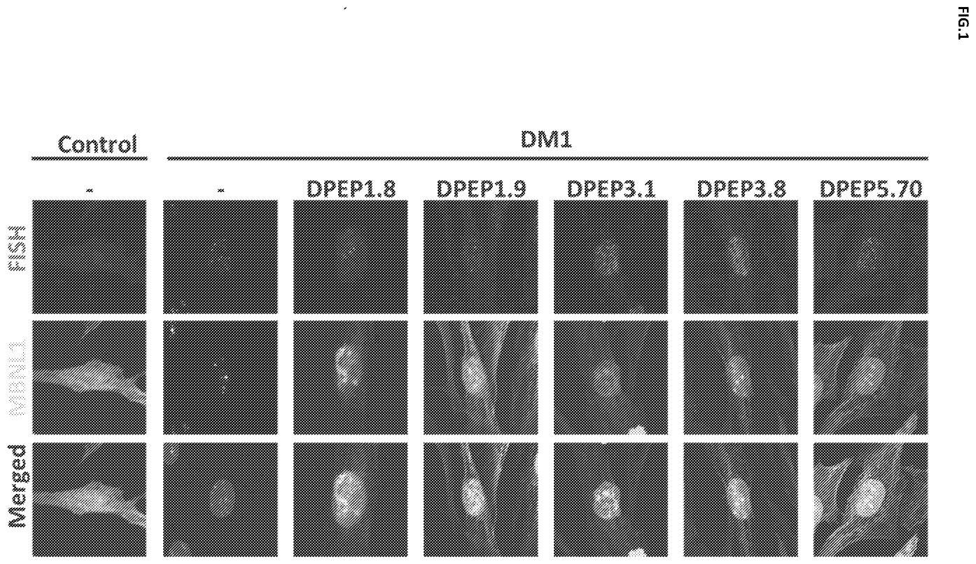

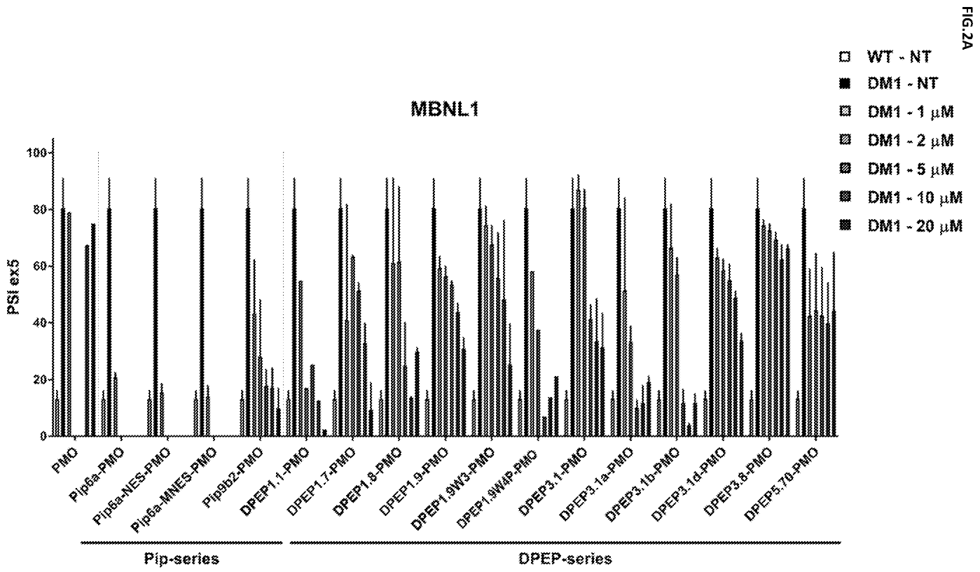

FIG. 1 shows a reduction in the number of pathogenic nuclear foci, and MBNL redistribution, in DM1 patient myoblasts with 2600 CTG repeats. Results are shown 48 hours after transfection at doses of different DPEP1/3-[CAG]₇ PMO conjugates that did not decrease cell viability of myoblasts or hepatocytes (showed at 10 uM).

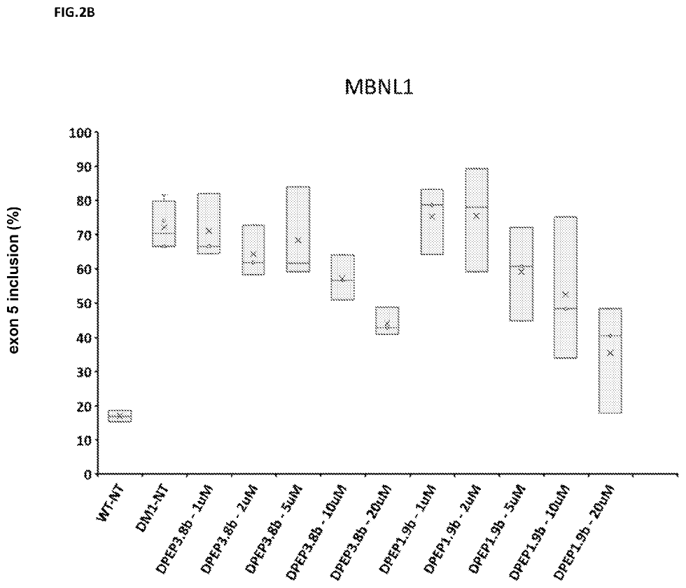

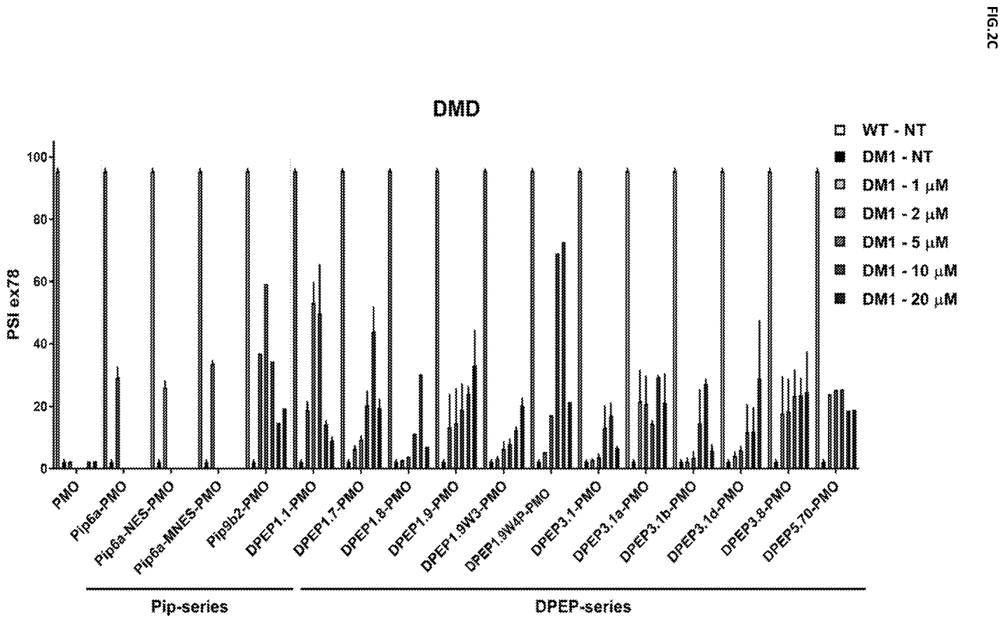

FIGS. 2A, B, C, D and E and FIG. 3A, B, C and D show different DPEP1/3-[CAG]₇ PMO conjugates correct splicing defects of Mbnl-dependent transcripts in DM1 patient myoblasts derived from DM1 patients with 2600 repeats in the DMPK gene at various concentrations, compared with conjugates formed with prior peptide carriers; Pip6a and Pip9b2.

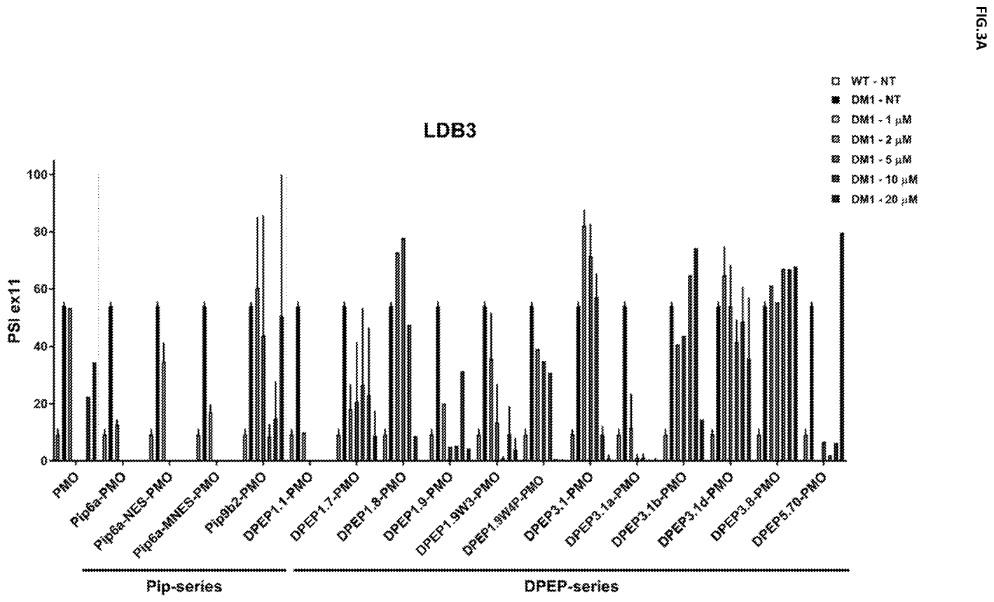

FIG. 4 shows systemic delivery of different DPEP1/3-[CAG]₇ PMO conjugates at 30 mg/kg (IV, tail vein) corrects splicing defects of Mbnl-dependent transcripts in gastrocnemius (gast.) and quadriceps (quad.) of HSA-LR mice. RT-PCR analyses of the splicing of cicnl exon 7a, serca exon22, and mbnll exon 5 (the most widely used DM1 biomarkers) show the splicing normalization to wild type levels for DPEP1 and 3 based conjugates. The data of 6 HSA-LR mice per peptide-PMO were analyzed by ANOVA and Tukey's post-test compared to untreated HSA-LR mice. Data are mean ±SEM (*p<0.05, **p<0.01, ***p<0.001, n.s. not significant).

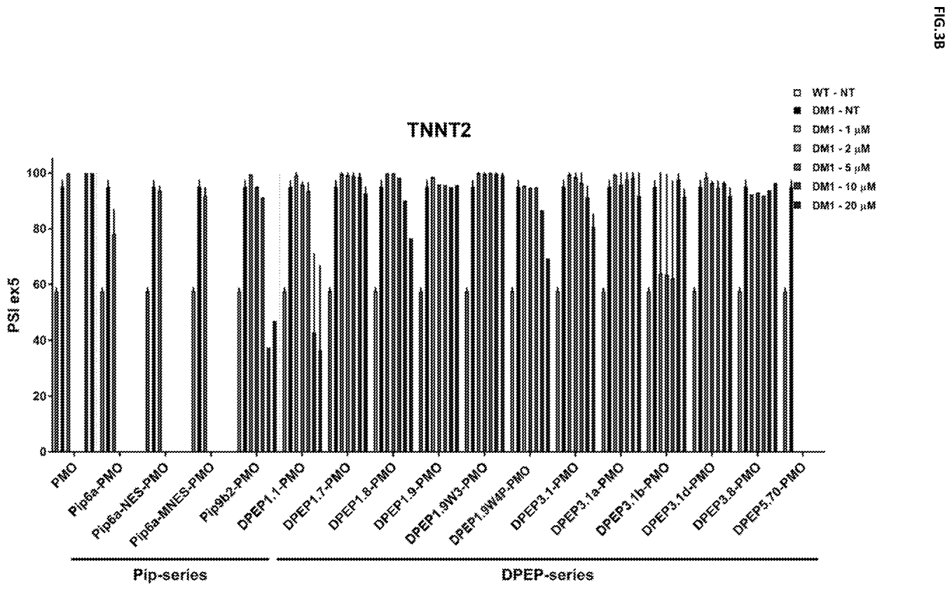

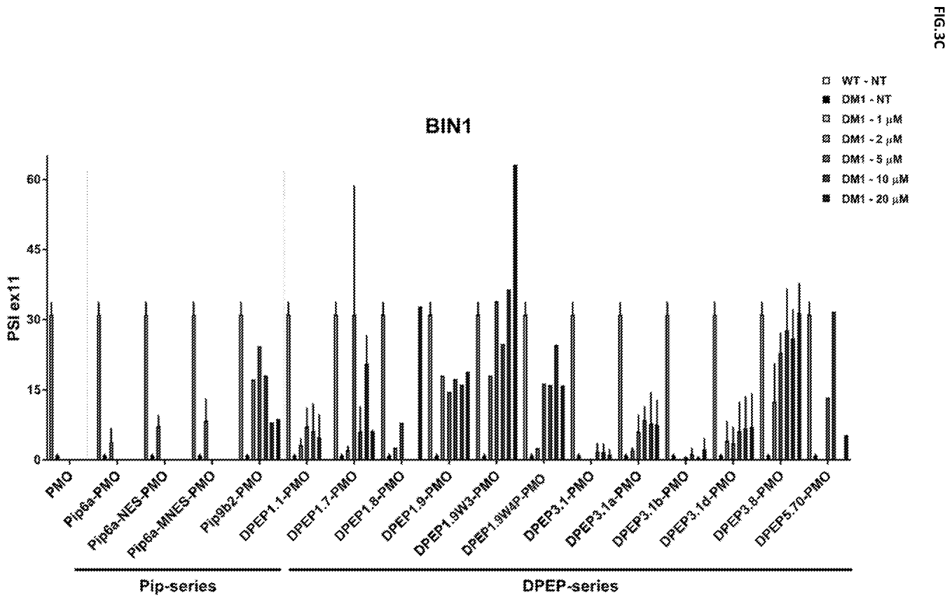

FIG. 5 shows the percentage myoblast cell viability after DM1 patient myoblasts with 2600 CTG repeats are 48 hours transfected with various doses of different DPEP1/3-[CAG]₇ PMO conjugates. DPEP1/3-[CAG]₇ PMO conjugate concentrations can be increased several fold from therapeutic levels without causing cell death in myoblasts, in contrast to conjugates formed with prior peptide carriers; Pip6a and Pip9b2.

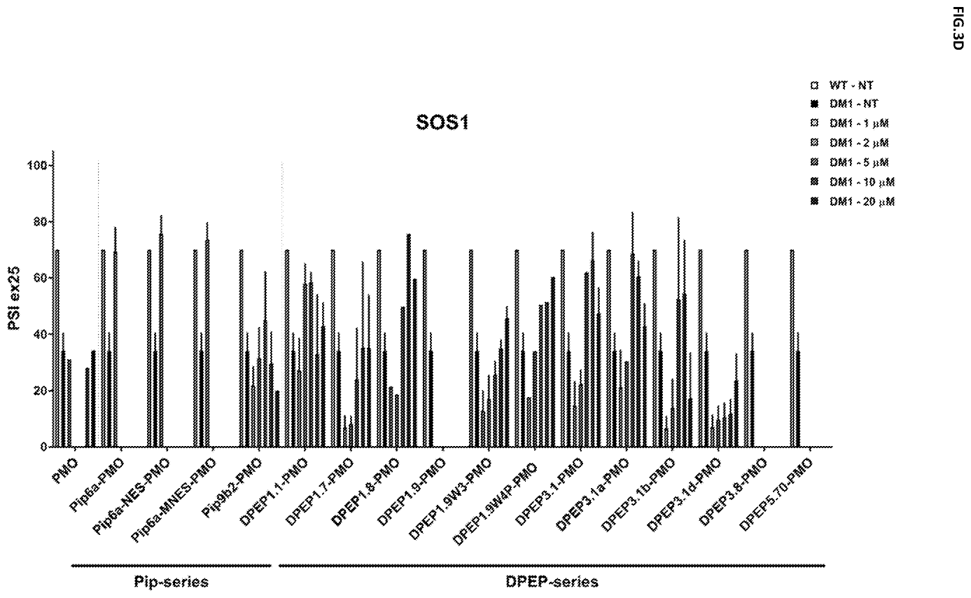

FIG. 6 shows the percentage hepatocyte cell viability after DM1 patient myoblasts with 2600 CTG repeats are 48 hours transfected with different DPEP1/3-[CAG]7 conjugates and comparative conjugates. DPEP1/3-[CAG]₇ PMO conjugate concentrations can be increased several fold from therapeutic levels without causing cell death in hepatocytes, in contrast to conjugates formed with prior peptide carriers; Pip6a and Pip9b2.

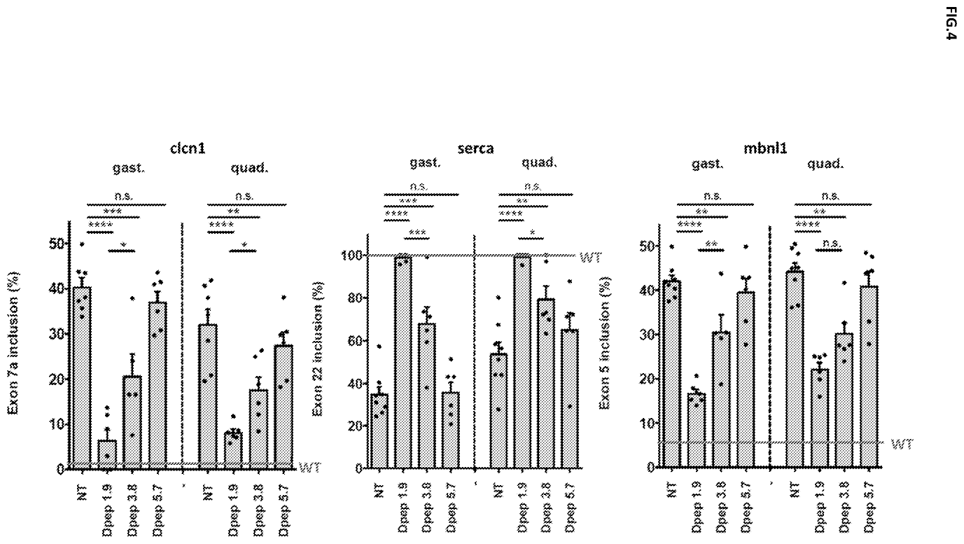

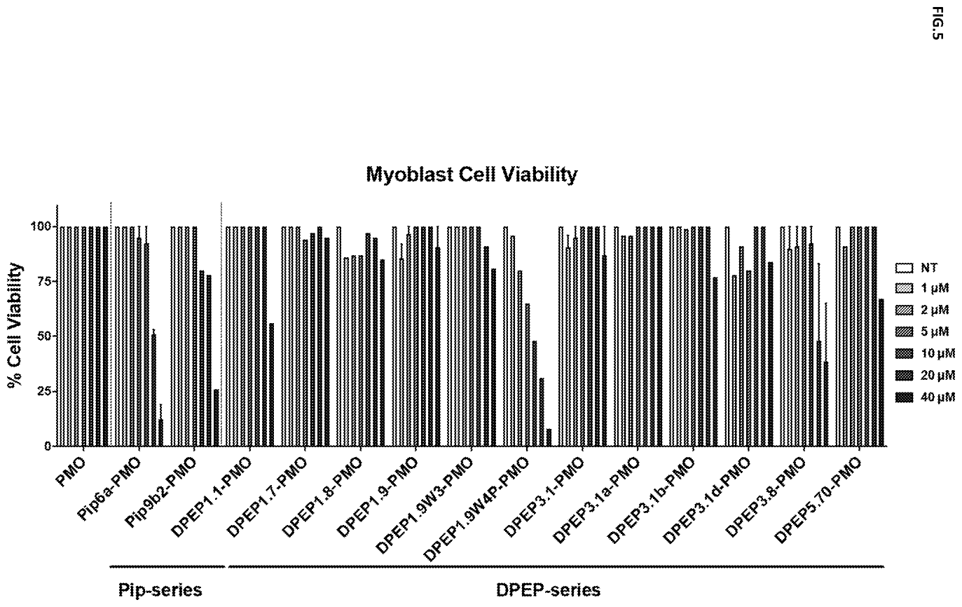

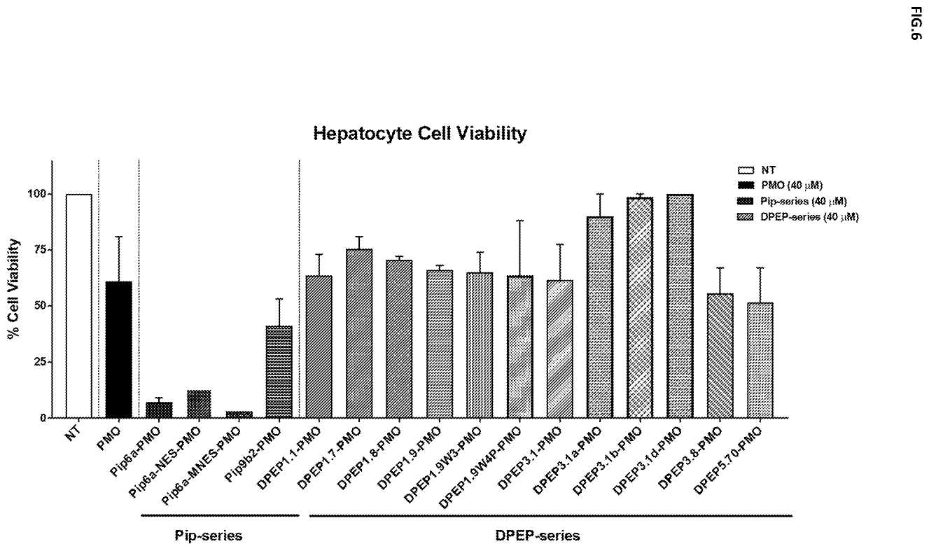

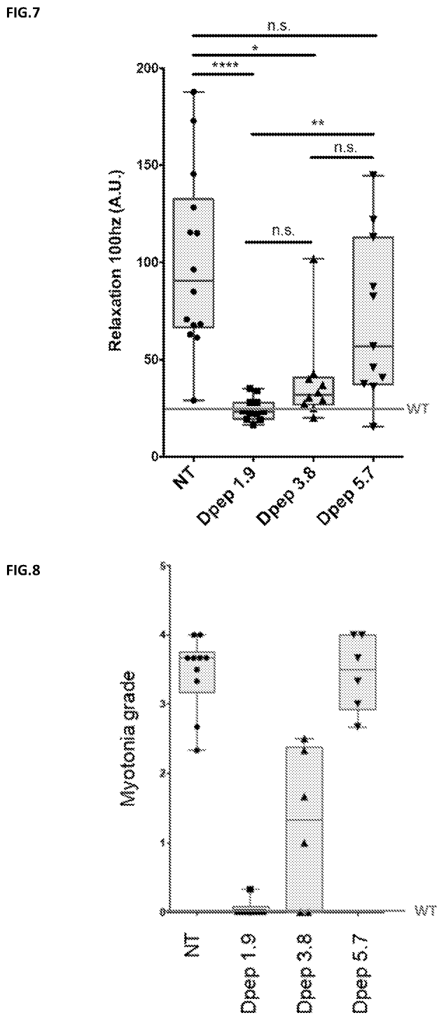

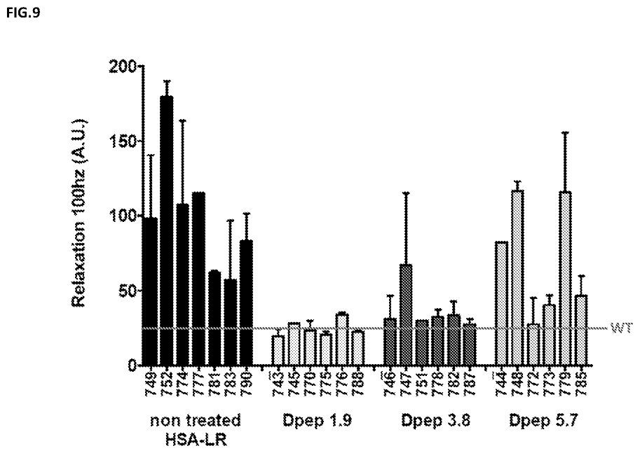

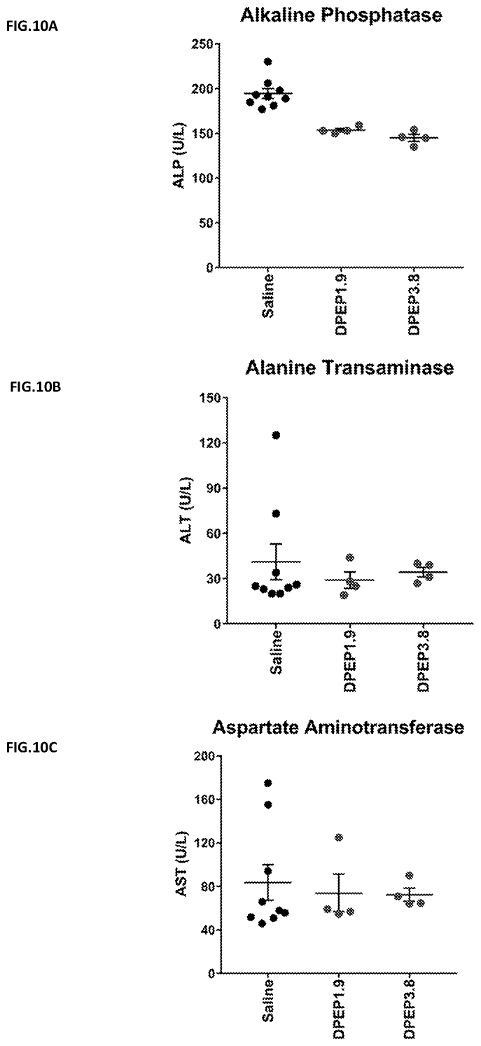

FIGS. 7 and 9 show electromyographic myotonia measurements in gastrocnemius muscles of HSA-LR mice 2 weeks after a single dose of different DPEP1/3-[CAG]₇ PMO conjugates (30mg/kg, n=6, IV, tail vein). The data were analyzed by ANOVA and Tukey's post-test compared to untreated HAS-LR mice and a comparative conjugate with DPEP5.7. Data are mean ±SEM (*p<0.05, **p<0.01, ***p<0.001, n.s. not significant). FIG. 10 shows the data detailed by individual tested.

FIG. 8 shows the corresponding myotonia grade measurements for the data in FIGS. 8 and 10 in HSA-LR mice 2 weeks after a single dose of different DPEP1/3-[CAG]₇ PMO conjugates (30mg/kg, n=6, IV, tail vein). The data were analyzed by unpaired Student's t test compared to untreated HSA-LR mice and a comparative conjugate with DPEP5.7. Data are mean ±SEM.

FIGS. 10A, B and C show ALP, ALT and AST levels assessed in serum from C57BL6 female mice (8-10 weeks age, n=5 per group), who were administered bolus IV (tail vein) injection of different DPEP1/3-[CAG]₇ PMO conjugates, at day 7 post-injection collection in serum compared to saline. ALP, ALT, AST levels were similar to saline control injections in comparison to the fold increases induced by the prior Pip series of peptide carriers.

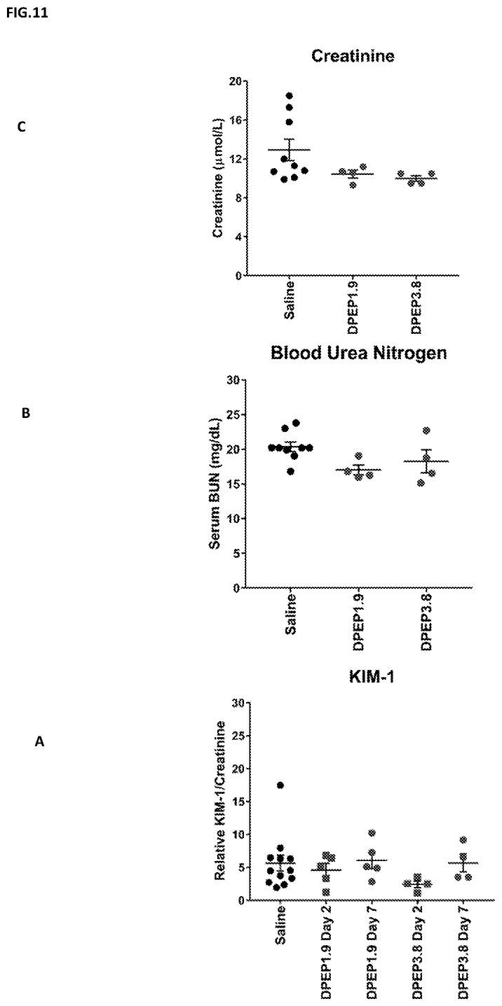

FIG. 11A shows KIM-1 levels assessed in serum assessed in urine from Day 2 and Day 7 post-injection of different DPEP1/3-[CAG]₇ PMO conjugates to C57BL6 female mice measured by ELISA (R&D cat# MKM100) with samples diluted to fit within standard curve. Values were normalised to urinary creatinine levels (Harwell) to account for urine protein concentration. KIM-1 levels were similar to saline control injections in comparison to the fold increases induced by the prior Pip series of peptide carriers.

FIGS. 11B and C show BUN and Creatinine levels assessed in serum from Day 7 post-injection of different DPEP1/3-[CAG]₇ PMO conjugates to C57BL6 female mice (Harwell) compared to saline. BUN and creatinine levels were similar to saline control injections in comparison to the fold increases induced by prior Pip series of peptide carriers.

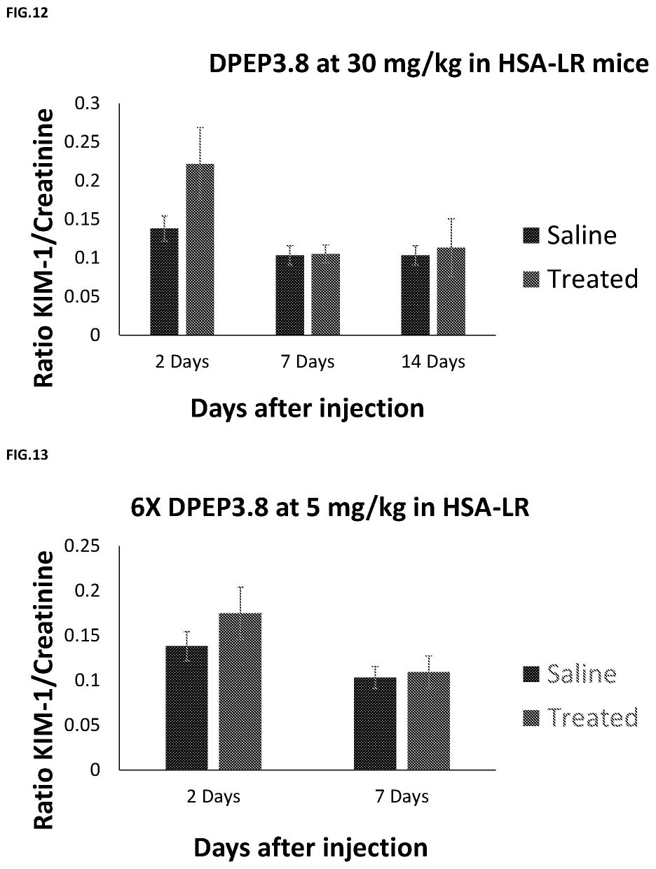

FIGS. 12 and 13 show the ratio of KIM-1/creatinine assessed in urine from day 2, 7 and 14 after administration of the DPEP3.8-[CAG]₇ PMO conjugate by injection to C57BL6 female mice at 30mg/kg or at 6 doses of 5mg/kg compared to saline. Creatinine and KIM-1 levels were similar to saline control injections in comparison to the fold increases induced by the prior Pip series of peptide carriers

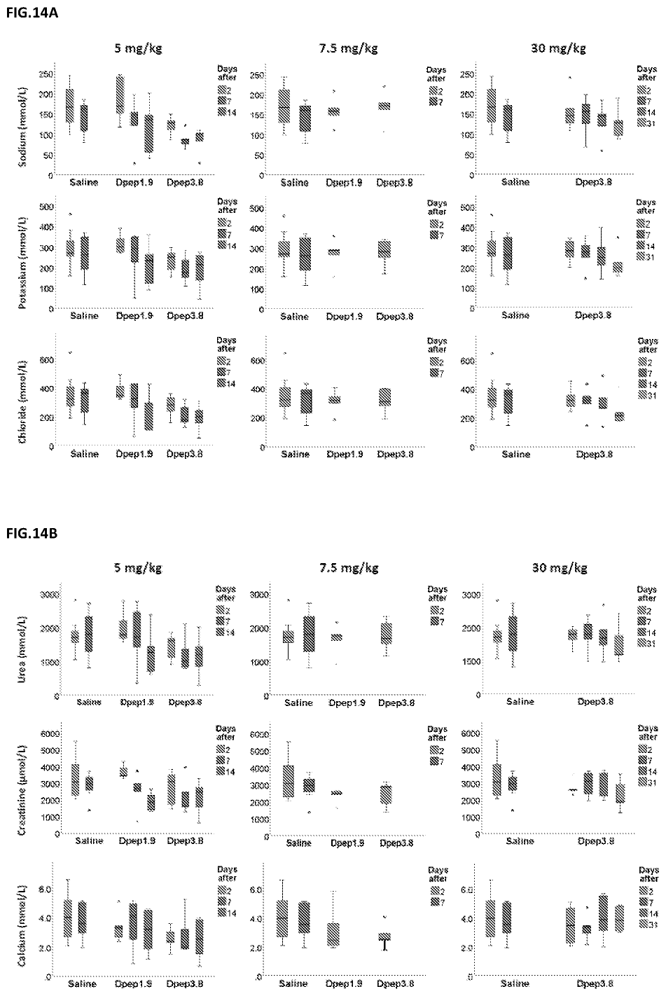

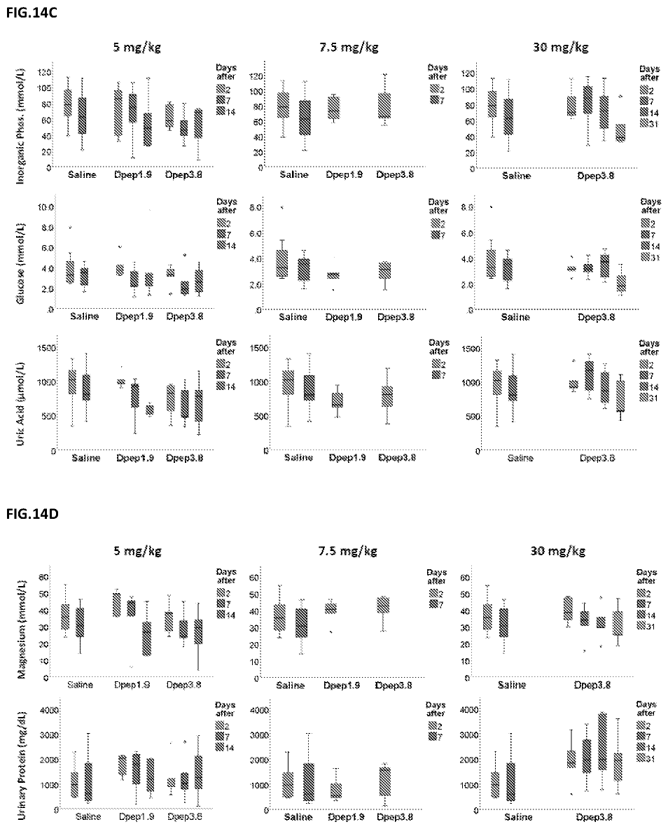

FIGS. 14A, B, C and D show urine sodium, potassium, chloride, urea, creatinine, calcium, phosphorous, glucose, uric acid, magnesium and protein levels in urine after different DPEP1/3-[CAG]₇ PMO conjugates were administered by injection to C57BL6 female mice (8-12 weeks age, n=5 per group) at 5, 7.5 and 30 mg/kg compared to saline. Error bars indicate SEM.

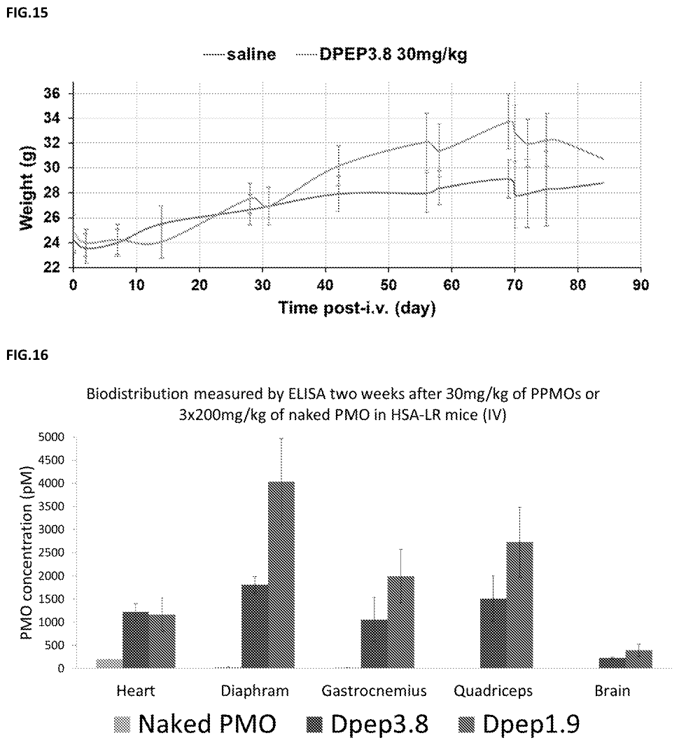

FIG. 15 shows HSA-LR mice weight after DPEP3.8-[CAG]₇ PMO conjugate treatment. Long term weight of 5 HSA-LR mice injected with a single dose of 30 mg/kg does not show any significant decrease when compared with 5 HSA-LR mice injected with saline.

FIG. 16 shows biodistribution delivery analysis of different DPEP1/3-[CAG]₇ PMO conjugates measured by ELISA two weeks after administration of 30 mg/kg of conjugate or 3×200 mg/kg of naked PMO in HSA-LR mice (IV). Evaluation of DPEP1.9 and DPEP3.8 conjugate biodistribution reveals optimal delivery to critically affected tissues in DM1. PMOs were detected by a custom ELISA assay using probes labelled with digoxigenin and biotin. Two weeks after treatments the concentration of PMO in muscle tissues was still >1nM vs the low pM detected after naked PMO injections (despite the >20-fold difference in molarity of naked PMO vs DPEP-PMO conjugate treatments) (n=4). Data are expressed as mean +/−SEM. Statistics: One-way ANOVA with Tukey post-test.

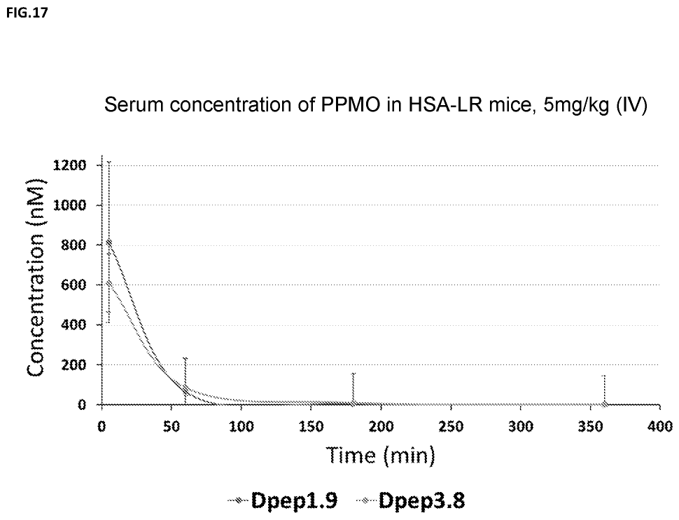

FIG. 17 shows the pharmacokinetic properties of different DPEP1/3-[CAG]₇ PMO conjugates measured in serum after a single dose at 5 mg/kg. Custom made ELISAs were used to quantify concentrations in serum reaching 500-800nM 5 min after IV injections at 5 mg/kg, dropping to 100 nM after 1 h and 10 nM after 3 hours. 6 h after the treatment concentrations were ˜1 nM with most of the compound being already cleared or delivered to the tissues of interest.

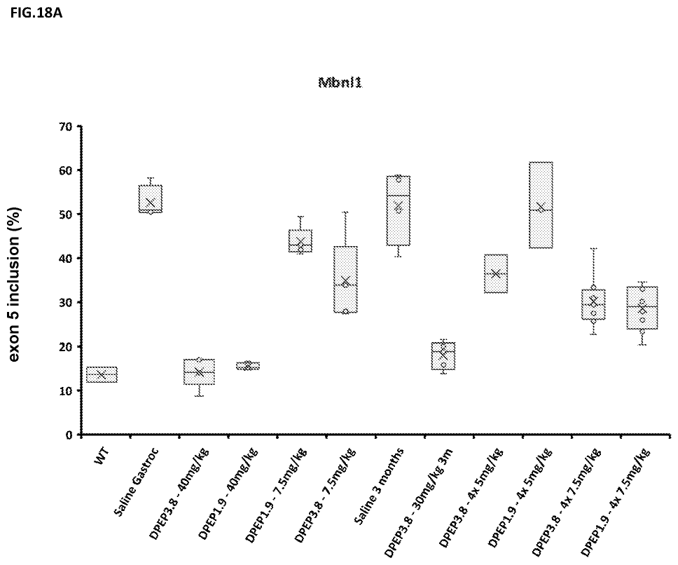

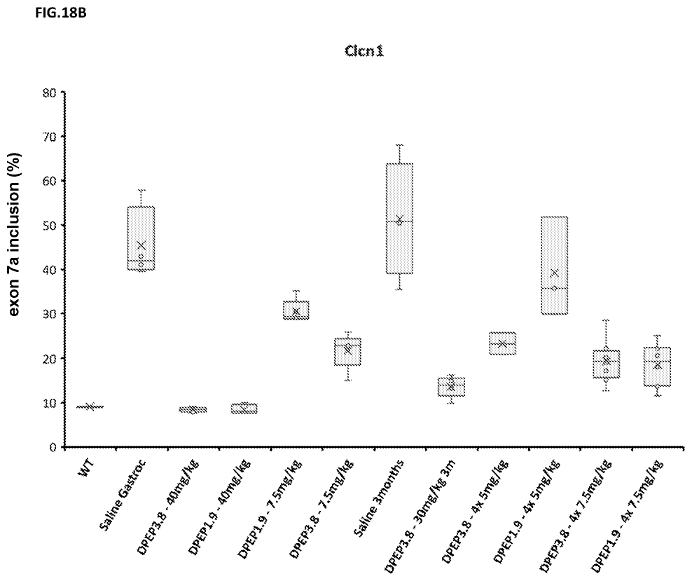

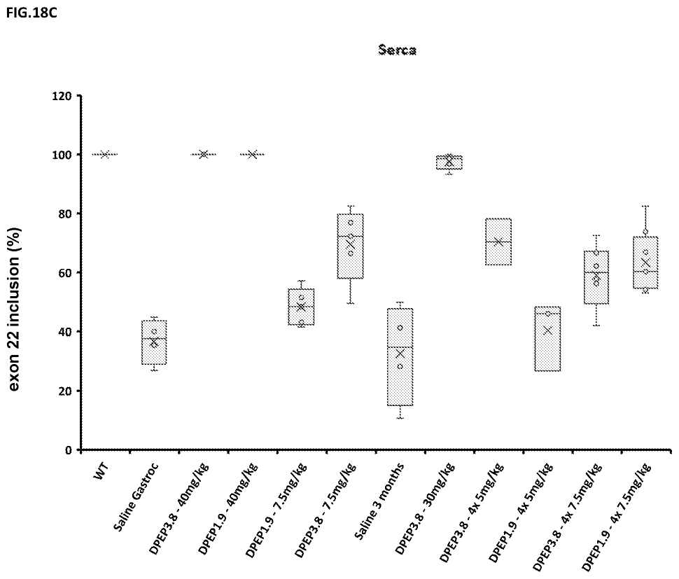

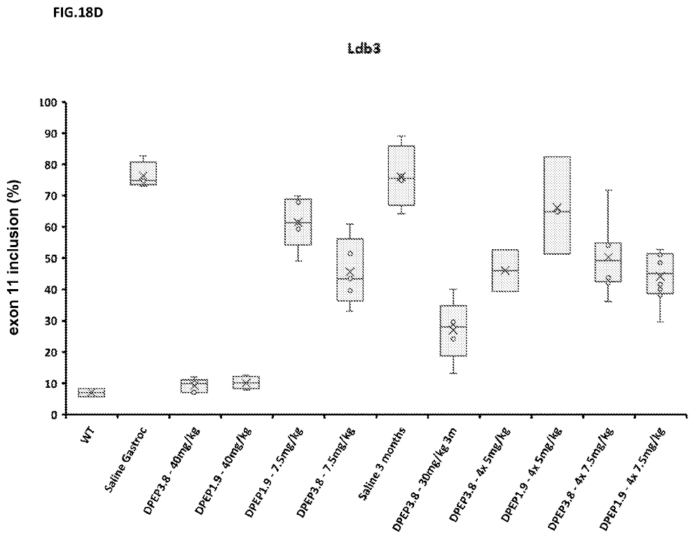

FIGS. 18A, B, C and D show in more detail that the systemic delivery of different DPEP1/3-[CAG]₇ PMO conjugates correct splicing defects of Mbnl-dependent transcripts in gastrocnemius of HSA-LR mice. RT-PCR analyses of the splicing of Clcnl exon 7a, Serca exon22, MbnI1 exon 5 and Ldb3 exon11 showed the splicing normalization to wild type levels with DPEP1.9 and DPEP3.8 based conjugates at 30 and 40 mg/kg. The splicing correction lasts for at least 3 months after treatment and it was also significant after single low doses (5 and 7.5 mg/kg) (boxes indicate distribution of data into quartiles, highlighting the mean, error bars indicate variability outside the upper and lower quartiles, n=5 per group).

FIGS. 19A, B and C show myotonia grade in HSA-LR mice is corrected to wild type levels (from 4 to 0) after 30 or 40 mg/kg single doses of DPEP3.8 and DPEP1.9 based conjugates. This correction lasts at least 3 months after treatment (A). When the dose is spread in four injections (4×7.5 mg/kg) myotonia is decreased to 50% (B), whereas lowering the dose to 4×5 mg/kg produces reductions of 20-25% two weeks after the last injection (C) (error bars indicate SEM); (n=6, IV, tail vein).

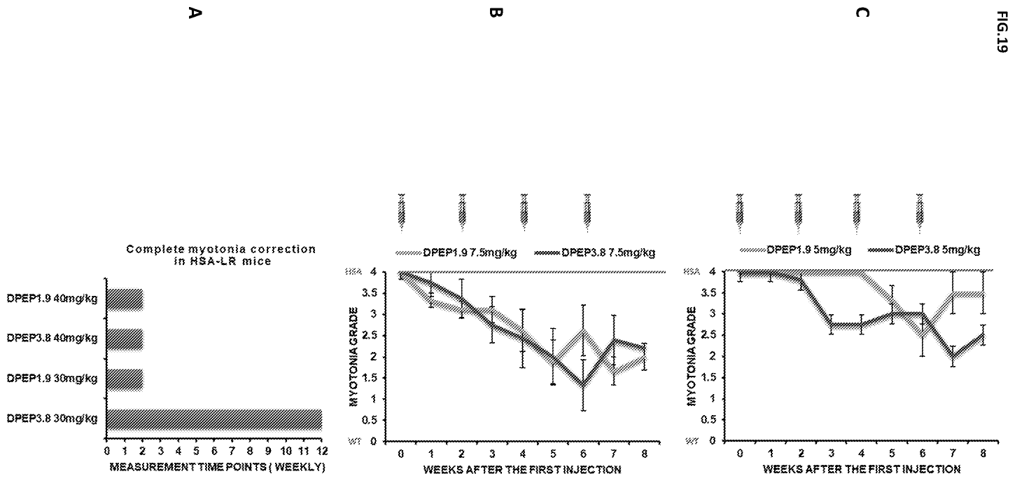

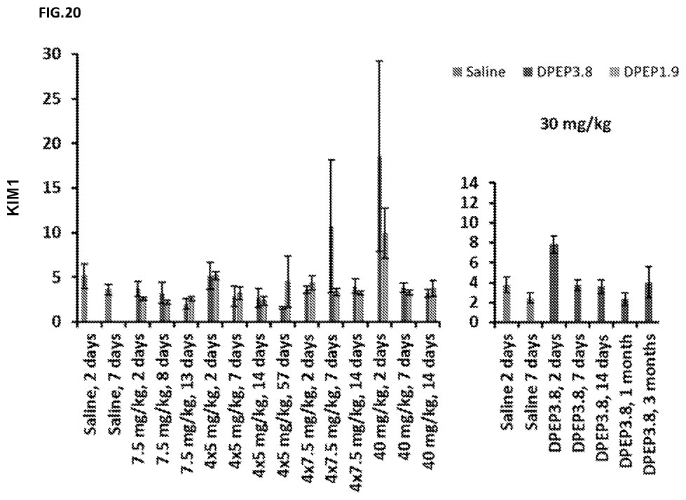

FIG. 20 shows toxicology screening in serum and urine 2 days and 1 week after the IV administration of different DPEP1/3-[CAG]₇ PMO conjugates in HSA-LR mice (8-12 weeks age, n=5 per group) results showed no significant changes at doses that were able to normalize the phenotype of HSA-LR mice. Only after treatments of DPEP1.9, DPEP3.8, DPEP3.1 and DPEP3.1b at 30 mg/kg or 40 mg/kg and only 2d after the treatment there was a significant change in KIM1 levels when compared with saline treated HSA-LR mice, error bars indicate SEM.

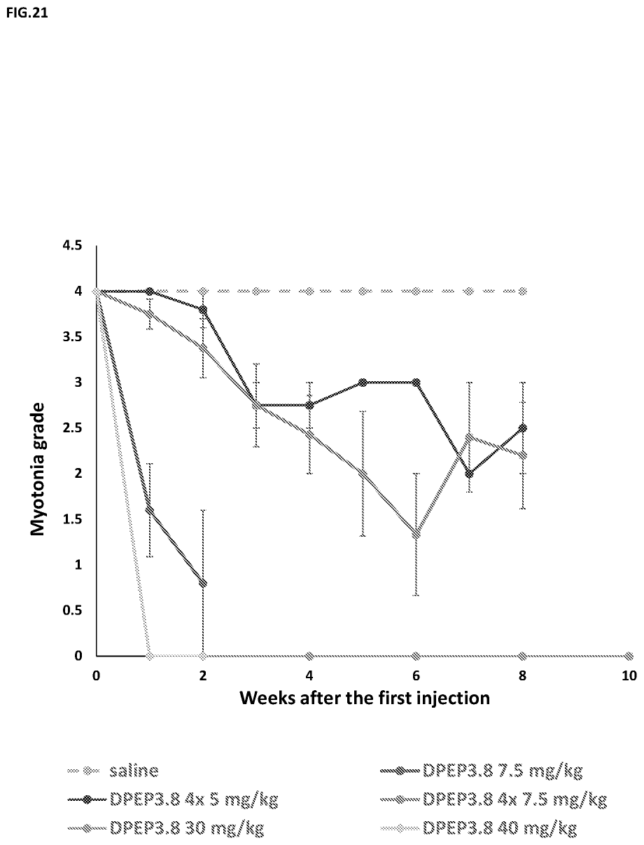

FIG. 21 shows the DM1 phenotype (myotonia) correction in HSA-LR mice over the course of a number of weeks after the first injection of various administration regimes including: 4 doses of 5 mg/kg of DPEP3.8-[CAG]₇ PMO conjugate, 4 doses of 7.5 mg/kg of DPEP3.8-[CAG]₇ PMO conjugate, a single 7.5mg/kg dose DPEP3.8-[CAG]₇ PMO conjugate, a single 30 mg/kg dose of DPEP3.8-[CAG]₇ PMO conjugate, or a single 40 mg/kg dose of DPEP3.8-[CAG]₇ PMO conjugate. Reductions in myotonia can be achieved after treatments with low doses of DPEP3.8-[CAG]₇ PMO conjugate (5-7.5 mg/kg) which are not associated with any toxicity.

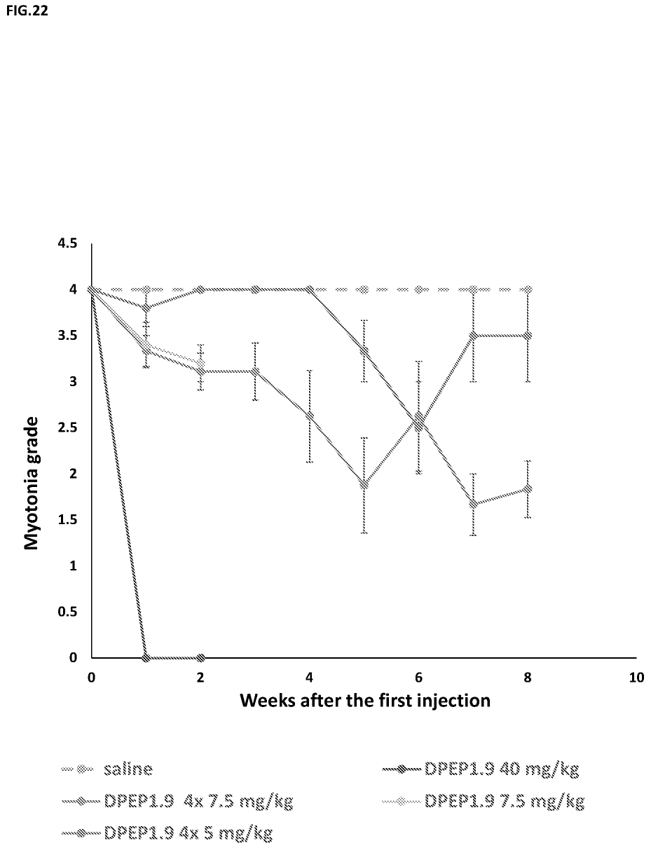

FIG. 22 shows the DM1 phenotype (myotonia) correction in HSA-LR mice over the course of a number of weeks after the first injection of various administration regimes including: 4 doses of 5 mg/kg of DPEP1.9-[CAG]₇ PMO conjugate, 4 doses of 7.5 mg/kg of DPEP1.9-[CAG]₇ PMO conjugate, a single 7.5 mg/kg dose DPEP1.9-[CAG]₇ PMO conjugate, or a single 40 mg/kg dose of DPEP1.9-[CAG]₇ PMO conjugate. Reductions in myotonia can be achieved after treatments with low doses of DPEP1.9-[CAG]₇ PMO conjugate (5-7.5 mg/kg) which are not associated with any toxicity.

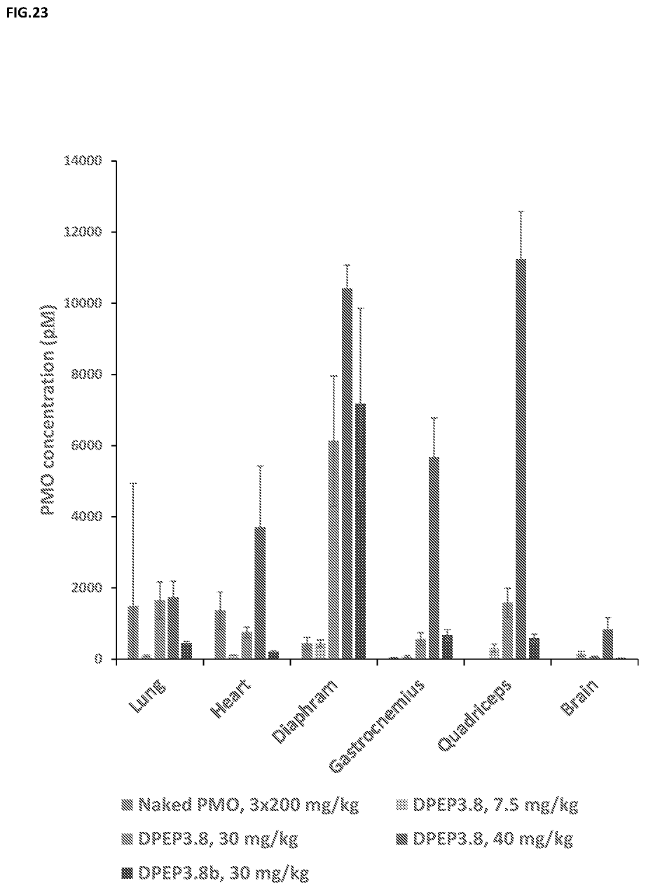

FIG. 23 shows the PMO concentration (pM) in various tissues 2 weeks after IV administration of naked PMO (3 doses of 200 mg/kg), DPEP3.8-[CAG]₇ PMO conjugate at 30 mg/kg, DPEP3.8b-[CAG]₇ PMO conjugate at 30 mg/kg, DPEP3.8-[CAG]₇ PMO conjugate at 7.5 mg/kg, and DPEP3.8-[CAG]₇ PMO conjugate at 40 mg/kg to HSA-LR mice. Both peptides (DPEP3.8 and DPEP3.8b) are able to deliver the PMO to muscle successfully, reaching concentrations of >6nM in skeletal muscle.

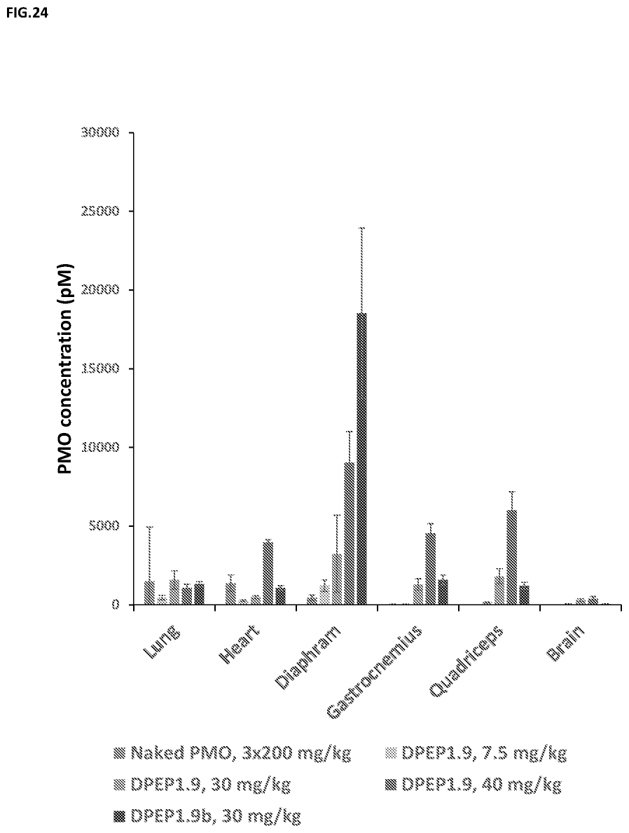

FIG. 24 shows the PMO concentration (pM) in various tissues 2 weeks after IV administration of naked PMO (3 doses of 200 mg/kg), DPEP1.9-[CAG]₇ PMO conjugate at 30 mg/kg, DPEP1.9b-[CAG]₇ PMO conjugate at 30 mg/kg, DPEP1.9-[CAG]₇ PMO conjugate at 7.5 mg/kg, and DPEP1.9-[CAG]₇ PMO conjugate at 40 mg/kg to HSA-LR mice. Both peptides (DPEP1.9 and DPEP1.9b) are able to deliver the PMO to muscle successfully. DPEP1.9b-[CAG]7 PMO is particularly good reaching Diaphragm (>15 nM two weeks after a single IV injection at 30 mg/kg).

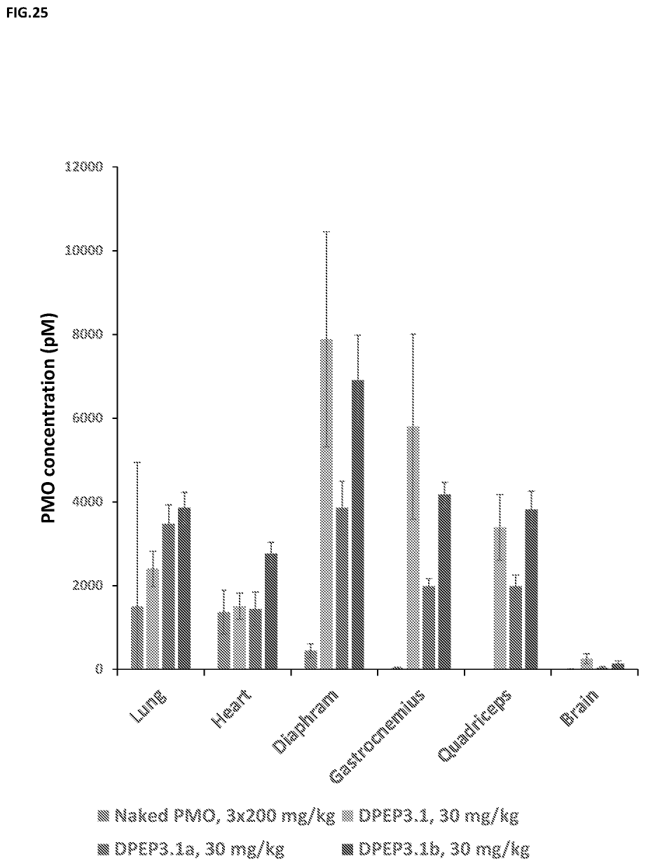

FIG. 25 shows the PMO concentration (pM) in various tissues 2 weeks after IV administration of naked PMO (3 doses of 200 mg/kg), DPEP3.1-[CAG]₇ PMO conjugate at 30 mg/kg, DPEP3.1a-[CAG]₇ PMO conjugate at 30 mg/kg, and DPEP3.1b-[CAG]₇ PMO conjugate at 30 mg/kg to HSA-LR mice. The three peptides (DPEP3.1, DPEP3.1a and DPEP3.1b) were able to deliver the PMO to both skeletal and cardiac muscle (>1 nM).

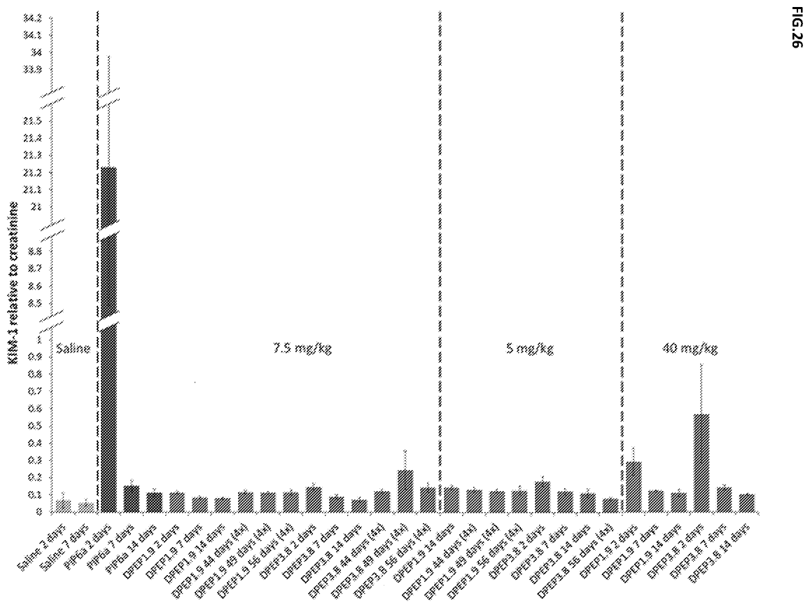

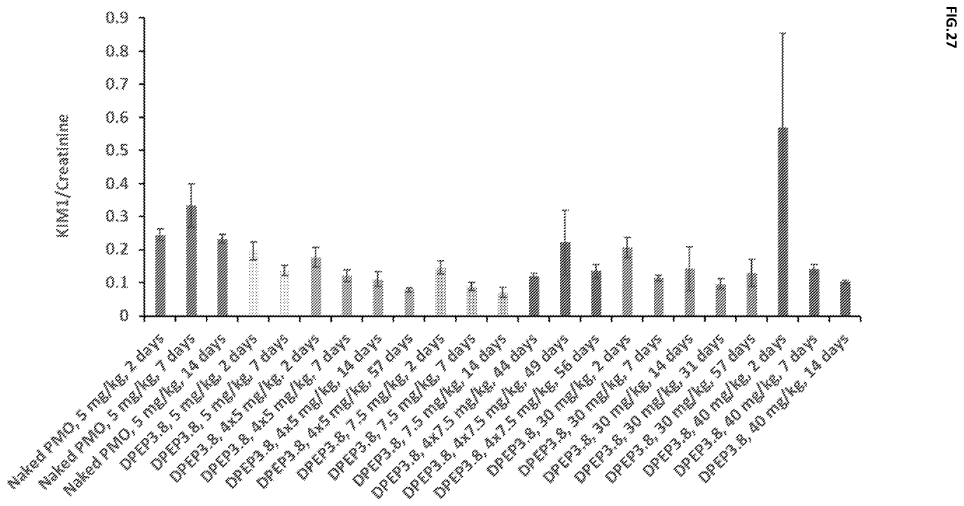

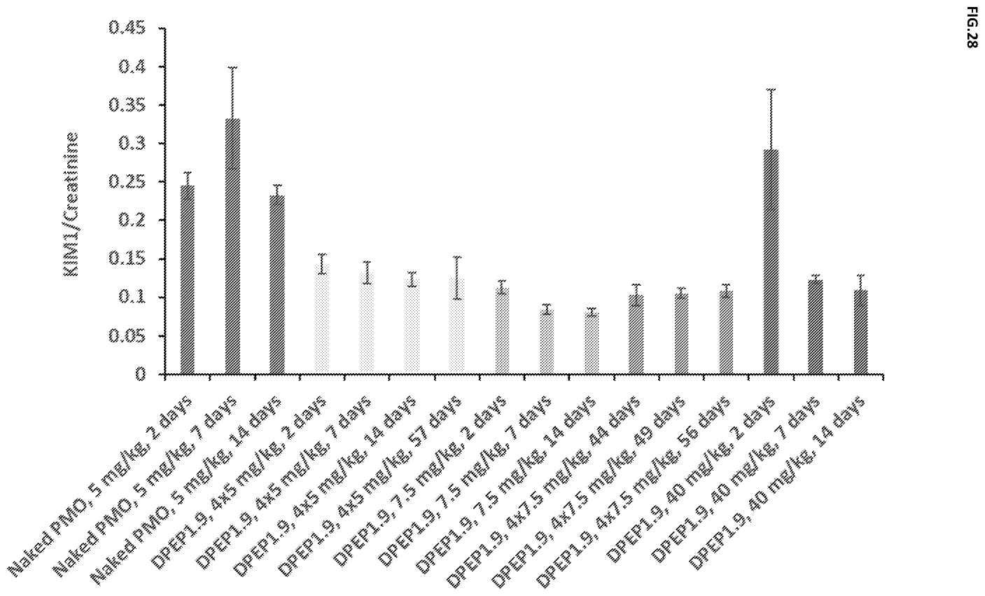

FIGS. 26, 27 and 28 show toxicology screens of KIM-1 relative to creatinine levels measured in urine at varying times after the systemic IV administration in HSA-LR mice of different peptide-[CAG]₇ PMO conjugates of the invention at different doses, compared to saline, compared to naked [CAG]7 PMO, and also compared to Pip peptide-[CAG]₇ PMO conjugates. The DPEP peptide-[CAG]₇ PMO conjugates of the invention retain low toxicity even at higher doses compared with especially the Pip6a-[CAG]7 PMO conjugate. DPEP conjugates do not impact toxicity biomarkers using dose regimes able to reverse DM1 phenotype to healthy levels.

Throughout the description and claims of this specification, the words “comprise” and “contain” and variations of them mean “including but not limited to”, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive.

The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed. The reader's attention is directed to all papers and documents which are filed concurrently with or previous to this specification in connection with this application and which are open to public inspection with this specification, and the contents of all such papers and documents are incorporated herein by reference.

### Examples

1. MATERIALS AND METHODS

P-PMO Synthesis and Preparation 9-Fluroenylmethoxycarbonyl (Fmoc) protected L-amino acids, benzotriazole-1-yl-oxy-tris-pyrrolidino-phosphonium (PyBOP), 2-(1H-benzotriazole-1-yl)-1,1,3,3-tetramethyluronium hexafluorophosphate (HBTU), and the Fmoc-β-Ala-OH preloaded Wang resin (0.19 or 0.46 mmol g⁻¹) were obtained from Merck (Hohenbrunn, Germany). 1-Hydroxy-7-azabenzotriazole (HOAt) was obtained from Sigma-Aldrich. HPLC grade acetonitrile, methanol and synthesis grade N-methyl-2-pyrrolidone (NMP) were purchased from Fisher Scientific (Loughborough, UK). Peptide synthesis grade N,N-dimethylformamide (DMF) and diethyl ether were obtained from VWR (Leicestershire, UK). Piperidine and trifluoroacetic acid (TFA) were obtained from Alfa Aesar (Heysham, England). PMO was purchased from Gene Tools Inc. (Philomath, USA). All other reagents were obtained from Sigma-Aldrich (St. Louis, Mo., USA) unless otherwise stated. MALDI-TOF mass spectrometry was carried out using a Voyager DE Pro BioSpectrometry workstation. A stock solution of 10 mg mL⁻¹ of a-cyano-4-hydroxycinnamic acid or sinapinic acid in 50% acetonitrile in water was used as matrix. Error bars are ±0.1%.

Synthesis of P-PMO Peptides For Screening

a) Preparation of a library of peptide variants

Peptides were either prepared on a 10 pmol scale using an Intavis Parallel Peptide Synthesizer or on a 100 pmol scale using a CEM Liberty Blue™ Peptide Synthesizer (Buckingham, UK) using Fmoc-β-Ala-OH preloaded Wang resin (0.19 or 0.46 mmol g⁻¹, Merck Millipore) by applying standard Fmoc chemistry and following manufacturer's recommendations. In the case of synthesis using the Intavis Parallel Peptide Synthesizer, double coupling steps were used with a PyBOP/NMM coupling mixture followed by acetic anhydride capping after each step. For synthesis using the CEM Liberty Blue Peptide Synthesizer, single standard couplings were implemented for all amino acids except arginine, which was performed by double couplings. The coupling was carried out once at 75° C. for 5 min at 60-watt microwave power except for arginine residues, which were coupled twice each. Each deprotection reaction was carried out at 75° C. twice, once for 30 sec and then for 3 min at 35-watt microwave power. Once synthesis was complete, the resin was washed with DMF (3×50 mL) and the N-terminus of the solid phase bound peptide was acetylated with acetic anhydride in the presence of DIPEA. at room temperature. After acetylation of the N-terminus, the peptide resin was washed with DMF (3×20 mL) and DCM (3×20 mL). The peptides were cleaved from the solid support by treatment with a cleavage cocktail consisting of trifluoroacetic acid (TFA): H₂O: triisopropylsilane (TIPS) (95%: 2.5%: 2.5%: 3-10 mL) for 3 h at room temperature. After peptide release, excess TFA was removed by sparging with nitrogen. The crude peptide was precipitated by the addition of cold diethyl ether (15-40 mL depending on scale of the synthesis) and centrifuged at 3200 rpm for 5 min. The crude peptide pellet was washed thrice with cold diethyl ether (3×15 mL) and purified by RP-HPLC using a Varian 940-LC HPLC System fitted with a 445-LC Scale-up module and 440-LC fraction collector. Peptides were purified by semi-preparative HPLC on an RP-C18 column (10×250 mm, Phenomenex Jupiter) using a linear gradient of CH₃CN in 0.1% TFA/H₂O with a flow rate of 15 mL min⁻¹. Detection was performed at 220 nm and 260 nm. The fractions containing the desired peptide were combined and lyophilized to yield the peptide as a white solid (see Table 1 for yields).

| | Sequence | Sequence Tested |
|---|---|---|
| Peptide | ID NO. | (with additional C and N |
| Number | incorporated | terminal modifications) |
| D-PEP1.1 | 27 | Ac-RBRRBRRFQILYRBRBR-B |
| D-PEP1.7 | 33 | Ac-RBRRBRFQILYRBRBR-B |
| D-PEP1.8 | 34 | Ac-RBRRBFQILYRBRRBR-B |
| D-PEP1.9 | 35 | Ac-RBRRBRFQILYBRBR-B |
| D-PEP1.9a | 35 | Ac-RBRRBRFQILYBRBR-Ab |
| D-PEP1.9b | 35 | Ac-RBRRBRFQILYBRBR-E |
| D-PEP1.9W3 | 93 | Ac-RBRRBRWWWBRBR-B |
| DPEP1.9W4P | 94 | Ac-RBRRBRWWPWWBRBR-B |
| D-PEP3.1 | 37 | Ac-RBRRBRRFQILYRBHBH-B |
| D-PEP3.1a | 37 | Ac-RBRRBRRFQILYRBHBH-Ab |
| D-PEP3.1b | 37 | Ac-RBRRBRRFQILYRBHBH-E |
| D-PEP3.1d | 37 | Succ-RBRRBRRFQILYRBHBH- |
| | | NH₂ |
| D-PEP3.8 | 44 | Ac-RBRRBRFQILYRBHBH-B |
| D-Pep3.8b | 44 | Ac-RBRRBRFQILYRBHBH-E |
| D-PEP5.70 | 97 | Ac-RBRBRS*RBRBR-B |
| P1p6a | 98 | Ac-RXRRBRRXR-YQFLI- |
| | | RXRBRXR-B |
| Pip9b2 | 99 | Ac-RXRRBRR-FQILY-RBRXR-B |

Table 1: peptides as synthesized for testing in the examples
with N-terminal acetylation (Ac), N-terminal succinic
acid linker (Succ), C-terminal β-alanine linker (B),
gamma-Aminobutyric acid linker (Ab) and glutamic acid
linker (E). S* is a glucosylated serine residue. Conjugates
formed with DPEP5.7, Pip6a and Pipb2 are comparative.

b) Synthesis of a library of Peptide-PMO conjugates

A 21-mer PMO antisense sequence for triplet repeat sequences (CAGCAGCAGCAGCAGCAGCAG (SEQ ID NO.95) otherwise known as [CAG]7 was used. The PMO sequence targeting CUG/CTG expanded repeats (5′-CAGCAGCAGCAGCAGCAGCAG-3′ (SEQ ID NO: 95)) was purchased from Gene Tools LLC. This is a [CAG]7 PMO as referenced elsewhere herein. The peptide was conjugated to the 3′-end of the PMO through its C-terminal carboxyl group. This was achieved using 2.5 and 2 equivalents of PyBOP and HOAt in NMP respectively in the presence of 2.5 equivalents of DIPEA and 2.5 fold excess of peptide over PMO dissolved in DMSO was used. In general, to a solution of peptide (2500 nmol) in N-methylpyrrolidone (NMP, 80 μL) were added PyBOP (19.2 μL of 0.3 M in NMP), HOAt in (16.7 μL of 0.3 M NMP), DIPEA (1.0 mL) and PMO (180 μL of 10 mM in DMSO). The mixture was left for 2.5 h at 40° C. and the reaction was quenched by the addition of 0.1% TFA in H₂O (300 μL). This solution was purified by Ion exchange chromatography using a converted Gilson HPLC system. The PMO-peptide conjugates were purified on an ion exchange column (Resource S 4 mL, GE Healthcare) using a linear gradient of sodium phosphate buffer (25 mM, pH 7.0) containing 20% CH₃CN. A sodium chloride solution (1 M) was used to elute the conjugate from the column at a flow rate of either 4 mL min⁻¹ or 6 mL min⁻¹. The fractions containing the desired compound were combined desalted immediately. The removal of excess salts from the peptide-PMO conjugate was afforded through the filtration of the fractions collected after ion exchange using an Amicon® ultra-15 3K centrifugal filter device. The conjugate was lyophilized and analyzed by MALDI-TOF. The conjugates were dissolved in sterile water and filtered through a 0.22 μm cellulose acetate membrane before use. The concentration of peptide-PMO was determined by the molar absorption of the conjugates at 265 nm in 0.1 N HCl solution. (see Table 2 for yields).

TABLE 2
Yields of P-PMO conjugates for cell culture analysis and in vivo
experiments (The yields are based on dried weight of the lyophilised
purified P-PMO. The purity for the P-PMOs is greater than 95%
as ascertained by normal phase HPLC at 220 nm and 260 nm.

| Peptide | Yield |
|---|---|
| D-Pep 1.1 | 36% |
| D-Pep 1.7 | 41% |
| D-pep 1.8 | 38% |
| D-Pep 1.9 | 40% |
| D-Pep 1.9b | 34% |
| D-Pep 1.9W3 | 43% |
| D-Pep 1.9W4P | 23% |
| D-Pep 3.1 | 31% |
| D-Pep 3.1a | 17% |
| D-Pep 3.1b | 25% |
| D-Pep 3.1d | 37% |
| D-Pep 3.8 | 36% |
| D-Pep 3.8b | 35% |
| D-Pep 5.70 | 31% |

Animal model and ASO injections. Experiments were carried out in the University of Oxford or in the “Centre d'études fonctionnelles” (Faculté de Médecine Sorbonne University) according to UK and French legislation respectively (Ethics committee approval #1760-2015091512001083v6). The intravenous injections in HSA-LR or C57BL/6 mice were performed by single or multiple administrations via the tail vein. Doses of 5, 7.5, 12.5, 30 or 40 mg/kg of peptide-PMO-CAG7 and 12.5 or 200 mg/kg of PMO were diluted in 0.9% saline and given at a volume of 5-6 μL/g of body weight. Multiple injections were done at 2 weeks apart. Myotonia was evaluated and tissues were harvested 2 weeks after the last injection. For long-term experiments, tissues were harvested 3 months after the injection. For toxicology measurements, tissues were harvested after 1 week. Urine was tested by ELISAs (R&D cat# MKM100) with samples diluted to fit within standard curve. Values were normalised to urinary creatinine levels (Harwell) to account for urine protein concentration

In situ myotonia I muscle relaxation measurement. The isometric contractile properties of gastrocnemius muscle were studied in situ. Mice were anesthetized with a solution of ketamine/xylasine (80 mg/kg and 15 mg/kg, respectively). The knee and foot were fixed with clamps and pins. The distal tendon of the gastrocnemius muscle was attached to a lever arm of a servomotor system (305B, Dual-Mode Lever). Data were recorded and analyzed using PowerLab system (4SP, ADInstruments) and software (Chart 4, ADInstruments). The sciatic nerve (proximally crushed) was stimulated by a bipolar silver electrode using a supramaximal (10-V) square wave pulse of 0.1 ms duration. Absolute maximal isometric tetanic force (P0) was measured during isometric contractions in response to electrical stimulation (frequency of 25 to 150 Hz, train of stimulation of 500 ms). Myotonia was measured as the delay of relaxation muscle after the measure of P0.

Cell culture and Peptide-PMO treatment. Immortalized myoblasts from healthy individual or DM1 patient with 2600 CTG repeats were cultivated in a growth medium consisting of a mix of M199:DMEM (1:4 ratio; Life technologies) supplemented with 20% FBS (Life technologies), 50 μg/ml gentamycin (Life technologies), 25 μg/ml fetuin, 0.5 ng/ml bFGF, 5 ng/ml EGF and 0.2 μg/ml dexamethasone (Sigma-Aldrich). Myogenic differentiation was induced by switching confluent cell cultures to DMEM medium supplemented with 5 μ/ml insulin (Sigma-Aldrich) for myoblasts. For treatment, WT or DM1 cells are differentiated for 4 days. Then, medium was changed with fresh differentiation medium with peptide-PMO conjugates at a 1, 2 ,5 10, 20 or 40 μM concentration. Cells were harvested for analysis 48 h after treatment. Cell viability was quantified in after 2 days of transfection of peptide-PMOs at 40 uM in human hepatocytes or at a 1, 2 ,5 10, 20 or 40 μM concentration in human myoblasts using a fluorescent-based assay (Promega).

RNA isolation, RT-PCR and qPCR analysis. For mice tissues: prior to RNA extraction, muscles were disrupted in TriReagent (Sigma-Aldrich) using Fastprep system and Lysing Matrix D tubes (MP biomedicals). For human cells: prior to RNA extraction, cells were lysed in a proteinase K buffer (500 mM NaCl, 10 mM Tris-HCl, pH 7.2, 1.5 mM MgCl2, 10 mM EDTA, 2% SDS and 0.5 mg/ml of proteinase K) for 45 min at 55° C. Total RNAs were isolated using TriReagent according to the manufacturer's protocol. One microgram of RNA was reverse transcribed using M-MLV first-strand synthesis system (Life Technologies) according to the manufacturer's instructions in a total of 20 μL. One microliter of cDNA preparation was subsequently used in a semi-quantitative PCR analysis according to standard protocol (ReddyMix, Thermo Scientific). Primers are shown in the following table 3:

TABLE 3

| Primer Name | SEQ ID NO. | Species/Gene/Exon | Sequence (5′-3′) |
|---|---|---|---|
| MbnILF | 100 | Mouse-Human/mbnI1/exon5 | GCTGCCCAATACCAGGTCAAC |
| MbnILR | 101 | Mouse-Human/mbnI1/exon5 | TGGTGGGAGAAATGCTGTATGC |
| Clcn1.F | 102 | Mouse/clcn1/exon7a | TTCACATCGCCAGCATCTGTGC |
| Clcn1.R | 103 | Mouse/clcn1/exon7a | CACGGAACACAAAGGCACTGAATGT |
| Serca.F | 104 | Mouse/serca1/ex0n22 | GCTCATGGTCCTCAAGATCTCAC |
| Serca.R | 105 | Mouse/serca1/exon22 | GGGTCAGTGCCTCAGCTTTG |
| Ldb3.F | 106 | Mouse/lbd3/exon11 | GGAAGATGAGGCTGATGAGTGG |
| Ldb3.R | 107 | Mouse/lbd3/exon11 | TGCTGACAGTGGTAGTGCTCTTTC |
| BIN.F | 108 | Human/BIN/exon11 | AGAACCTCAATGATGTGCTGG |
| BIN.R | 109 | Human/BIN/exon11 | TCGTGTTGACTCTGATCTCGG |
| DMD.F | 110 | Human/DMD/exon78 | TTAGAGGAGGTGATGGAGCA |
| DMD.R | 111 | Human/DMD/exon78 | GATACTAAGGACTCCATCGC |
| INSR.F | 112 | Human/INSR/exon11 | CCAAAGACAGACTCTCAGAT |
| INSR.R | 113 | Human/INSR/exon11 | AACATCGCCAAGGGACCTGC |
| LDB3.F | 114 | Human/LDB3/exon11 | GCAAGACCCTGATGAAGAAGCTC |
| LDB3.R | 115 | Human/LDB3/exon11 | GACAGAAGGCCGGATGCTG |
| SERCA.F | 116 | Human/SERCA/exon22 | ATCTTCAAGCTCCGGGCCCT |
| SERCA.R | 117 | Human/SERCA/exon22 | CAGCTCTGCCTGAAGATGTG |
| SOS1.F | 118 | Human/SOS1/exon25 | CAGTACCACAGATGTTTGCAGTG |
| SOS1.R | 119 | Human/SOS1/exon25 | TCTGGTCGTCTTCGTGGAGGAA |
| TNNT2.F | 120 | Human/TNNT2/exon5 | ATAGAAGAGGTGGTGGAAGAGTAC |
| TNNT2.R | 121 | Human/TNN2/exon5 | GTCTCAGCCTCTGCTTCAGCATCC |

PCR amplification was carried out for 25-35 cycles within the linear range of amplification for each gene. PCR products were resolved on 1.5-2% agarose gels, ethidium bromide-stained and quantified with ImageJ software. The ratios of exon inclusion were quantified as a percentage of inclusion relative to total intensity of isoform signals. To quantify the mRNA expression, real-time PCR was performed according to the manufacturer's instructions. PCR cycles were a 15-min denaturation step followed by 50 cycles with a 94° C. denaturation for 15 s, 58° C. annealing for 20 s and 72° C. extension for 20 s.

Fluorescent in situ hybridization/immunofluorescence. Fluorescent in situ hybridization (FISH) experiments were done as previously described (6) using a Cy3-labeled 2′OMe (CAG)7 probe (Eurogentec). For combined FISH-Immunofluorescence experiments, immunofluorescence staining was done after FISH last washing with a rabbit polyclonal anti-MBNL1 antibody followed by a secondary Alexa Fluor 488-conjugated goat anti-rabbit (1:500, Life technologies) antibody.

ELISA based measurements of oligonucleotide concentrations in tissues. Customized Hybridization-Based ELISAs were developed to determine the concentration of PMO oligonucleotides using phosphorothioate probes having phosphorothioate linkages (Sequence (5′->3′) [DIG]C*T*G*C*T*G*C*TGCTGCT*G*C*T*G*C*T*G[BIO] (SEQ ID NO:96)) double-labelled with digoxigenin and biotin. The assay had a linear detection range of 5-250 pM (R2>0.99) in mouse serum and tissue lysates. The probe was used to detect peptide-PMOs or naked PMO concentrations in eight different tissues (brain, kidney, liver, lung, heart, diaphragm, gastrocnemius and quadriceps) from treated HSA-LR mice.

2. RESULTS

In this work, we used an arginine-rich cell-penetrating peptide having specific structure and showed that such a peptide conjugated to a [CAG]7 morpholino phosphorodiamidate oligomer (PMO) dramatically enhanced ASO delivery into striated muscles of DM1 model HSA-LR mice following systemic administration in comparison to the unconjugated PMO and other peptide carrier conjugate strategies. Thus, low dose treatment of a conjugate formed of peptide-[CAG]₇ PMO as claimed herein targeting pathologic expansions was sufficient to reverse both splicing defects and myotonia in DM1 mice (HSA-LR) and normalized the overall disease-transcriptome. Moreover, treated DM1 patient derived muscle cells (myoblasts) showed that the peptide-[CAG]₇ PMO conjugates as claimed herein specifically target mutant CUGexp-DMPK transcripts to abrogate the detrimental sequestration of MBNL1 splicing factor by nuclear RNA foci and consequently MBNL1 functional loss, responsible for splicing defects and muscle dysfunction. Our results demonstrate that the peptide-[CAG]₇ PMO conjugates as claimed herein induce high efficacy and long-lasting correction of DM1-associated phenotypes at both molecular and functional levels, and strongly support the use of these peptide-conjugates for systemic corrective therapy in DM1.

We have produced data with conjugates comprising peptide carriers which contain no artificial amino acids, such as X residues, that have wider therapeutic window and safer toxicology profile than previous cell penetrating peptides and, therefore, constitute more promising candidates to be tested in DM1 patients. These new generation of so called ‘DPEP1 and DPEP3’ peptides have shown high efficacy in reducing the number of pathogenic foci (FIG. 1) and in correcting splicing defects in vitro when conjugated to a CAG7 repeat antisense oligonucleotide PMO (FIGS. 2, 3, 4, and 19). None of the concentrations tested caused reductions of cell viability in human hepatocytes (1-40 μM) contrary to a similar comparative conjugate formed from a known ‘Pip’ carrier peptides; Pip6a-PMO and Pip9b2-PMO that induced significant cell mortality (>50%) at 40 μM (FIG. 7). Many of the concentrations tested caused no reduction in cell viability of human myoblasts, and fared better compared to similar comparative conjugates formed from known ‘Pip’ carrier peptides Pip6a-PMO and Pip9b2-PMO that induced cell mortality at lower doses (FIGS. 5 and 6).

Subsequently, we tested if these new peptides were also active to correct myotonia and splicing changes in HSA-LR mice. To do so we tested the leading peptide carriers of the DPEP 1 and 3 series DPEP1.9 and DPEP3.8 in comparison with a prior peptide carrier DPEP 5.70.

We were able to show that splicing defects (FIG. 4) and myotonia (FIGS. 8, 9, and 10) are corrected to wild type levels two weeks after 30 mg/kg treatments of conjugates formed with both DPEP3.8 and DPEP1.9.

Biodistribution of naked PMO versus conjugates formed with carrier peptides DPEP1.9 and DPEP3.8 was assessed by ELISA to quantify delivery of peptide-[CAG]₇ PMO conjugate. Detection of PMO in critically affected tissues in DM1, such as heart and brain, is important for drug delivery development. A single intravenous injection of peptide-[CAG]₇ PMO conjugate at 30 mg/kg or 3 injections at 200 mg/kg of naked PMO were administered to HAS-LR mice (total 600 mg/kg). Gastrocnemius, quadriceps, diaphragm, heart and brain were analysed for PMO detection 2 weeks post administration. The unconjugated naked [CAG]7 PMO has low to non-detectable levels in all tissues tested, however the [CAG]7 PMO conjugated to peptide carriers DPEP1.9 and DPEP3.8 was detected at higher levels despite being injected at lower doses (>20 fold molarity). In general peptide-[CAG]₇ PMO conjugates were detected in quadriceps, gastrocnemius and diaphragm at 1 nM-4 nM and in heart at 1 nM 2 weeks after 30 mg/kg injections (FIG. 17).

TABLE 4

| | Naked PMO | Dpep3.8 | Dpep1.9 |
|---|---|---|---|
| pM in Tissue | 600 mg/kg | 30 mg/kg | 30 mg/kg |
| Kidney | 426748 | 443553 | 1142917 |
| Liver | 252 | 24295 | 101072 |
| Lung | 113 | 3956 | 6215 |
| Heart | 198 | 1225 | 1167 |
| Diaphragm | 30 | 1808 | 4033 |
| Gastrocnemius | 18 | 1057 | 1999 |
| Quadriceps | ND | 1513 | 2727 |
| Brain | ND | 226 | 394 |

We also studied the pharmacokinetic properties of peptide-[CAG]₇ PMO conjugates of the invention measured in serum after low doses of peptide-[CAG]₇ PMO conjugates (5 mg/kg). We quantified concentrations in serum reaching 500-800 nM 5min after IV injections, dropping to 100 nM after 1h and 10 nM after 3 hours. 6 h after the treatment concentrations were -1nM with most of the compound being already cleared or delivered to the tissues of interest (FIG. 18).

The preliminary toxicology evaluation of conjugates formed with DPEP3.8 and DPEP1.9 carrier peptides in wild type mice indicated that ALP, ALT, AST, KIM-1, creatinine, BUN and NGAL levels were similar to saline control injections, in contrast to the fold increases typically induced by currently available peptide carriers from the Pip series. With this preliminary data we showed that conjugates formed from DPEP peptides with a [CAG]7 PMO are as active as Pip6a in vivo yet have wider therapeutic window (FIGS. 11, 12 and 21).

Additionally, weight of 5 HSA-LR mice injected with a single dose of a conjugate formed from DPEP3.8-[CAG]₇ at 30 mg/kg did not show any significant trend when compared with 5 HSA-LR mice injected with saline (FIG. 16).

Furthermore, recovery times of HSA-LR mice after injections with DPEP based [CAG]7 PMO conjugates are shorter than after injection with conjugates formed with prior peptide carriers such as Pip6a (Table 5).

TABLE 5

| Summary of recovery times after injection with peptide-PM0CAG7 | mouse | age | | time AV ± SD | |
|---|---|---|---|---|---|
| DPEP1.9 6X 5 mg/kg repeated injections | HSA-LR | 8-12 | weeks | 0 | min |
| DPEP3.8 6X 5 mg/kg repeated injections | HSA-LR | 8-12 | weeks | 0 | min |
| DPEP1.9 4X 7.5 mg/kg repeated injections | HSA-LR | 8-12 | weeks | 0 | min |
| DPEP3.8 4X 7.5 mg/kg repeated injections | HSA-LR | 8-12 | weeks | 0 | min |
| DPEP1.9 7.5 mg/kg | HSA-LR | 8-12 | weeks | 0 | min |
| DPEP3.8 7.5 mg/kg | HSA-LR | 8-12 | weeks | 0 | min |
| DPEP1.9 30 mg/kg | WT | 8-12 | weeks | 17.5 min ± 2.5 | |
| DPEP1.9b 30 mg/kg | WT | 8-12 | weeks | 15 | min |
| DPEP3.8 30 mg/kg | WT | 8-12 | weeks | 7.5 min ± 2.5 | |
| DPEP3.1a 30 mg/kg | WT | 8-12 | weeks | 10 | min |
| DPEP3.8 30 mg/kg | HSA-LR | 8-12 | weeks | 60 min ± 10 | |
| DPEP1.9 40 mg/kg | HSA-LR | 8-12 | weeks | 57.5 min ± 26 | |
| DPEP3.8 40 mg/kg | HSA-LR | 8-12 | weeks | 60 min ± 15.5 | |
| DPEP3.8 30 mg/kg | HSA-LR | 30 | weeks | 60 | min |
| DPEP1.9 30 mg/kg | HSA-LR | 30 | weeks | >60 | min |
| pip6a 12.5 mg/kg | HSA-LR | 8-12 | weeks | >60 | min |

Evaluating the efficacy of the conjugates of the invention in more in detail we also found that splicing defects and myotonia are corrected to wild type levels for at least 3 months (FIGS. 19 and 20 respectively) after administration of DPEP peptide-[CAG]₇ PMO conjugates. We also measured 50% reduction of missplicing and myotonia after 7.5 mg/kg doses.

Notably, conjugates formed with prior peptide carriers such as Pip6a-[CAG]7 PMO cannot be tested at >20 mg/kg without causing high rates of mortality in mice, this is contrary to the conjugates of the invention for which the concentration can be increased more than 5-fold without causing any mortality. Furthermore, in the toxicity screening we only detected changes from saline levels with doses of more than 30 mg/kg in Kim1 levels 2d after treatment (FIG. 21).

The efficacy and toxicology data indicate that conjugates formed with carrier peptides of the DPEP1 and DPEP3 series as claimed are especially active blocking the sequestration of MBNL1 by the expanded CTG repeats in individuals affected by DM1, and induce low toxicity. These conjugates are able to completely correct the DM1 phenotype both at molecular level with the normalization of splicing and at muscle level with the correction of myotonia to wild type levels. These new conjugates further have wider therapeutic windows than conjugates formed with previous peptide carriers and, therefore, they are closer to realisation in the clinic.

To sum up, we show strong evidence supporting (1) that peptide-[CAG]₇ PMO block the pathological interactions of MBNL1 with the nuclear mutant CUGexp-RNA and rescue the downstream effects on RNA-splicing; (2) that the peptide conjugated antisense oligonucleotide approach allows the treatment to be delivered to inaccessible tissues like heart in diaphragm; (3) that the strong effect of the [CAG]7 PMO directly targeting the disease mutation combined with the ability of the peptide carrier technology to deliver the treatment in vivo with high efficacy converges on the powerful reversal of the DM1 phenotype in skeletal muscle DM1 mice (HSA-LR) to wild type levels even months after the treatment was discontinued. These pieces of evidence strongly suggest that peptide-[CAG]₇ conjugates are likely to have a strong disease modifying effect in DM1.

In fact, our experiments show that the effect that we observe in the HSA-LR mice is not only preventing the worsening of the DM1 pathology but that it is actually causing reversal of the disease phenotype. The expanded CUG-transcripts are already expressed in pups, and HSA-LR mice have significant myotonia present by the age of 1 month. The animals we used to generate the results supporting this application were treated at the age of at least 2 months and even 7 months, well beyond the point at which the molecular and functional phenotype of DM1 develops.

3. CONCLUSIONS

conjugates comprising DPEP carrier peptides and a [CAG]7 PMO (10pM) are able to reduce >50% the number of nuclear foci (at doses that did not decreased cell viability) in DM1 patient myoblasts and controls. None of the concentrations tested caused reductions of cell viability (1-40 μM) contrary to comparative conjugates formed with other carrier peptides that induced significant cell mortality (>50%) at 20pM or higher concentrations.

conjugates comprising DPEP carrier peptides and a [CAG]7 PMO showed positive pharmacokinetics and biodistribution evaluation revealed optimal delivery to critically affected tissues in DM1.

conjugates comprising DPEP carrier peptides and a [CAG]7 PMO induced splicing corrections of 50%-90% in Clcnl exon 7a, Serca exon22, MbnI1 exon 5 and Ldb3 exon11 at a dose (30 mg/kg, IV) that is less toxic than 12.5 mg/kg of comparative conjugates formed with other carrier peptides in HSA-LR mice. RT-PCR analyses also show the splicing normalization to wild type levels with conjugates comprising DPEP1.9 and DPEP3.8 at 30 and 40 mg/kg. The splicing correction lasts for at least 3 months after treatment and it was also significant after single low doses (5 and 7.5 mg/kg).

conjugates comprising DPEP carrier peptides and a [CAG]7 PMO decreased myotonia to wild type levels after a single injection at 40 mg/kg or 30 mg/kg (IV) according to myotonia qualitative observations and electromyographic myotonia measurements. Moderate correction of myotonia also occurred after 4 injections at 7.5 mg/kg of conjugates comprising DPEP3.8 or DPEP1.9.

* conjugates comprising DPEP carrier peptides and a [CAG]7 PMO injected at 30 mg/kg (IV) induced shorter lethargy times in wild type mice than a single injection of comparative conjugates formed with other carrier peptides at 12.5 mg/kg (>1 hr). Urine biochemistry tests for kidney function and blood analysis show no changes in comparison with saline in wild type mice and mild changes in HSA-LR Kim1 levels and urinary protein after =>30 mg/kg.

1. A conjugate comprising: a peptide carrier covalently linked to a therapeutic molecule;
wherein the peptide carrier has a total length of 40 amino acids or less and comprises: two or more cationic domains each comprising at least 4 amino acid residues and one or more hydrophobic domains each comprising at least 3 amino acid residues, wherein the peptide carrier does not contain artificial amino acid residues;
and wherein the therapeutic molecule comprises a nucleic acid, wherein the nucleic acid comprises a plurality of trinucleotide repeats.

2. The conjugate according to claim 1, wherein the nucleic acid comprises a plurality of trinucleotide repeats selected from GTC, CAG, GCC, GGC, CTT, and CCG repeats.

3. The conjugate according to claim 1 or 2, wherein the nucleic acid comprises a plurality of CAG repeats.

4. The conjugate according to any preceding claim, wherein the nucleic acid comprises between 5-20 trinucleotide repeats, preferably between 5-10 trinucleotide repeats, preferably 7 trinucleotide repeats.

5. The conjugate according to any preceding claim, wherein the nucleic acid binds to a trinucleotide repeat expansion.

6. The conjugate according to any preceding claim, wherein the peptide carrier consists of natural amino acid residues.

7. The conjugate according to any preceding claim, wherein each cationic domain has length of between 4 and 12 amino acid residues, preferably between 4 and 7 amino acid residues.

8. The conjugate according to any preceding claim, wherein each cationic domain comprises at least 40%, at least 45%, at least 50% cationic amino acids.

9. The conjugate according to any preceding claim, wherein each cationic domain comprises arginine, histidine, beta-alanine, hydroxyproline and/or serine residues, preferably wherein each cationic domain consists of arginine, histidine, beta-alanine, hydroxyproline and/or serine residues.

10. The conjugate according to any preceding claim, wherein the peptide carrier comprises two cationic domains.

11. The conjugate according to any preceding claim, wherein each cationic domain comprises one of the following sequences: RBRRBRR (SEQ ID NO:1), RBRBR (SEQ ID NO:2), RBRR (SEQ ID NO:3), RBRRBR (SEQ ID NO:4), RRBRBR (SEQ ID NO:5), RBRRB (SEQ ID NO:6), BRBR (SEQ ID NO:7), RBHBH (SEQ ID NO:8), HBHBR (SEQ ID NO:9), RBRHBHR (SEQ ID NO:10), RBRBBHR (SEQ ID NO:11), RBRRBH (SEQ ID NO:12), HBRRBR (SEQ ID NO:13), HBHBH (SEQ ID NO:14), BHBH (SEQ ID NO:15), BRBSB (SEQ ID NO:16), BRB[Hyp]B (SEQ ID NO:17), R[Hyp]H[Hyp]HB (SEQ ID NO:18), R[Hyp]RR[Hyp]R (SEQ ID NO:19) or any combination thereof;
preferably wherein each cationic domain consists of one the following sequences: RBRRBRR (SEQ ID NO:1), RBRBR (SEQ ID NO:2), RBRR (SEQ ID NO:3), RBRRBR (SEQ ID NO:4), RRBRBR (SEQ ID NO:5), RBRRB (SEQ ID NO:6), BRBR (SEQ ID NO:7), RBHBH (SEQ ID NO:8), HBHBR (SEQ ID NO:9), RBRHBHR (SEQ ID NO:10), RBRBBHR (SEQ ID NO:11), RBRRBH (SEQ ID NO:12), HBRRBR (SEQ ID NO:13), HBHBH (SEQ ID NO:14), BHBH (SEQ ID NO:15), BRBSB (SEQ ID NO:16), BRB[Hyp]B (SEQ ID NO:17), R[Hyp]H[Hyp]HB (SEQ ID NO:18), R[Hyp]RR[Hyp]R (SEQ ID NO:19) or any combination thereof.

12. The conjugate according to any preceding claim wherein each hydrophobic domain has a length of between 3-6 amino acids, preferably each hydrophobic domain has a length of 5 amino acids.

13. The conjugate according to any preceding claim wherein each hydrophobic domain comprises a majority of hydrophobic amino acid residues, preferably each hydrophobic domain comprises at least 70%, at least 75%, at least 80%, at least 85%, at least 90%, at least 95%, 100% hydrophobic amino acids.

14. The conjugate according to any preceding claim wherein each hydrophobic domain comprises phenylalanine, leucine, Isoleucine, tyrosine, tryptophan, proline, and glutamine residues; preferably wherein each hydrophobic domain consists of phenylalanine, leucine, isoleucine, tyrosine, tryptophan, proline, and/or glutamine residues.

15. The conjugate according to any preceding claim wherein the peptide carrier comprises one hydrophobic domain.

16. The conjugate according to any preceding claim wherein the or each hydrophobic domain comprises one of the following sequences: YQFLI (SEQ ID NO:20), FQILY (SEQ ID NO:21), ILFQY (SEQ ID NO:22), FQIY (SEQ ID NO:23), WWW, WWPWW (SEQ ID NO:24), WPWW (SEQ ID NO:25), WWPW (SEQ ID NO:26) or any combination thereof; preferably wherein the or each hydrophobic domain consists of one of the following sequences: YQFLI (SEQ ID NO:20), FQILY (SEQ ID NO:21), ILFQY (SEQ ID NO:22), FQIY (SEQ ID NO:23), WWW, WWPWW (SEQ ID NO:24), WPWW (SEQ ID NO:25), WWPW (SEQ ID NO:26) or any combination thereof.

17. The conjugate according to any preceding claim, wherein the peptide carrier consists of two cationic domains and one hydrophobic domain, preferably wherein the peptide consists of one hydrophobic core domain flanked by two cationic arm domains.

18. The peptide according to any preceding claim, wherein the peptide carrier consists of one of the following sequences: RBRRBRFQILYBRBR (SEQ ID NO:35), RBRRBRRFQILYRBHBH (SEQ ID NO:37), and RBRRBRFQILYRBHBH (SEQ ID NO:44).

19. The conjugate according to any preceding claim, wherein the peptide carrier is covalently linked to the therapeutic molecule by a linker.

20. The conjugate according to claim 19 wherein the linker is selected from G, BC, XC, C, GGC, BBC, BXC, XBC, X, XX, B, BB, BX, XB, E, GABA and succinic acid.

21. A conjugate according to any of claims 1-20 for use as a medicament.

22. A conjugate according to any of claims 1-20 for use in the prevention or treatment of a trinucleotide repeat disorder.

23. A conjugate for use according to claim 22 wherein the trinucleotide repeat disorder is selected from a polyglutamine disease or a non-polyglutamine disease.

24. A conjugate for use according to claim 22 or 23 wherein the trinucleotide repeat disorder is selected from: DRPLA (Dentatorubropallidoluysian atrophy), HD (Huntingdon's disease), HDL2 (Huntingdon disease like syndrome 2), SBMA (spinal and bulbar muscular atrophy), SCA1 (spinocerebellar ataxia type 1), SCA2 (spinocerebellar ataxia type 2), SCA3 (spinocerebellar ataxia type 3 or Machado-Jospeh disease), SCA6 (spinocerebellar ataxia type 6), SCA7 (spinocerebellar ataxia type 7), SCA17 (spinocerebellar ataxia type 17), HDL2 (Huntingdon disease like syndrome 2), FRAXA (Fragile X syndrome), FXTAS (Fragile X temor/ataxia syndrome), FRAXE (Fragile XE mental retardation), FRDA (Friedrich's ataxia), DM1 (Myotonic dystrophy type 1), SCA8 (spinocerebellar ataxia type 8), and SCA12 (spinocerebellar ataxia type 12).

25. A conjugate for use according to any of claims 22-24, wherein the trinucleotide repeat disorder is myotonic dystrophy type 1 (DM1).


SEQUENCE LISTING (121 sequences)

SEQ ID NO: 1 - protein, 7 aa, Artificial Sequence
  peptide
  MOD_RES (2)..(2): X is bAla
  MOD_RES (5)..(5): X is bAla
RXRRXRR

SEQ ID NO: 2 - protein, 5 aa, Artificial Sequence
  peptide
  MOD_RES (2)..(2): X is bAla
  MOD_RES (4)..(4): X is bAla
RXRXR

SEQ ID NO: 3 - protein, 4 aa, Artificial Sequence
  peptide
  MOD_RES (2)..(2): X is bAla
RXRR

SEQ ID NO: 4 - protein, 6 aa, Artificial Sequence
  peptide
  MOD_RES (2)..(2): X is bAla
  MOD_RES (5)..(5): X is bAla
RXRRXR

SEQ ID NO: 5 - protein, 6 aa, Artificial Sequence
  peptide
  MOD_RES (3)..(3): x is bAla
  MOD_RES (5)..(5): x is bAla
RRXRXR

SEQ ID NO: 6 - protein, 5 aa, Artificial Sequence
  peptide
  MOD_RES (2)..(2): x is bAla
  MOD_RES (5)..(5): x is bAla
RXRRX

SEQ ID NO: 7 - protein, 4 aa, Artificial Sequence
  peptide
  MOD_RES (1)..(1): x is bAla
  MOD_RES (3)..(3): x is bAla
XRXR

SEQ ID NO: 8 - protein, 5 aa, Artificial Sequence
  peptide
  MOD_RES (2)..(2): x is bAla
  MOD_RES (4)..(4): x is bAla
RXHXH

SEQ ID NO: 9 - protein, 5 aa, Artificial Sequence
  peptide
  MOD_RES (2)..(2): x is bAla
  MOD_RES (4)..(4): x is bAla
HXHXR

SEQ ID NO: 10 - protein, 7 aa, Artificial Sequence
  peptide
  MOD_RES (2)..(2): x is bAla
  MOD_RES (5)..(5): x is bAla
RXRHXHR

SEQ ID NO: 11 - protein, 7 aa, Artificial Sequence
  peptide
  MOD_RES (2)..(2): x is bAla
  MOD_RES (4)..(5): x is bAla
RXRXXHR

SEQ ID NO: 12 - protein, 6 aa, Artificial Sequence
  peptide
  MOD_RES (2)..(2): x is bAla
  MOD_RES (5)..(5): x is bAla
RXRRXH

SEQ ID NO: 13 - protein, 6 aa, Artificial Sequence
  peptide
  MOD_RES (2)..(2): x is bAla
  MOD_RES (5)..(5): x is bAla
HXRRXR

SEQ ID NO: 14 - protein, 5 aa, Artificial Sequence
  peptide
  MOD_RES (2)..(2): x is bAla
  MOD_RES (4)..(4): x is bAla
HXHXH

SEQ ID NO: 15 - protein, 4 aa, Artificial Sequence
  peptide
  MOD_RES (1)..(1): x is bAla
  MOD_RES (3)..(3): x is bAla
XHXH

SEQ ID NO: 16 - protein, 5 aa, Artificial Sequence
  peptide
  MOD_RES (1)..(1): x is bAla
  MOD_RES (3)..(3): x is bAla
  MOD_RES (5)..(5): x is bAla
XRXSX

SEQ ID NO: 17 - protein, 5 aa, Artificial Sequence
  peptide
  MOD_RES (1)..(1): x is bAla
  MOD_RES (3)..(3): x is bAla
  MOD_RES (4)..(4): x is hydroxyproline
  MOD_RES (5)..(5): x is bAla
XRXXX

SEQ ID NO: 18 - protein, 6 aa, Artificial Sequence
  peptide
  MOD_RES (2)..(2): x is hydroxyproline
  MOD_RES (4)..(4): x is hydroxyproline
  MOD_RES (6)..(6): x is bAla
RXHXHX

SEQ ID NO: 19 - protein, 6 aa, Artificial Sequence
  peptide
  MOD_RES (2)..(2): x is hydroxyproline
  MOD_RES (5)..(5): x is hydroxyproline
RXRRXR

SEQ ID NO: 20 - protein, 5 aa, Artificial Sequence
  peptide
YQFLI

SEQ ID NO: 21 - protein, 5 aa, Artificial Sequence
  peptide
FQILY

SEQ ID NO: 22 - protein, 5 aa, Artificial Sequence
  peptide
ILFQY

SEQ ID NO: 23 - protein, 4 aa, Artificial Sequence
  peptide
FQIY

SEQ ID NO: 24 - protein, 5 aa, Artificial Sequence
  peptide
WWPWW

SEQ ID NO: 25 - protein, 4 aa, Artificial Sequence
  peptide
WPWW

SEQ ID NO: 26 - protein, 4 aa, Artificial Sequence
  peptide
WWPW

SEQ ID NO: 27 - protein, 17 aa, Artificial Sequence
  peptide
  MOD_RES (2)..(2): x is bAla
  MOD_RES (5)..(5): x is bAla
  MOD_RES (14)..(14): x is bAla
  MOD_RES (16)..(16): x is bAla
RXRRXRRFQILYRXRXR

SEQ ID NO: 28 - protein, 16 aa, Artificial Sequence
  peptide
  MOD_RES (2)..(2): x is bAla
  MOD_RES (5)..(5): x is bAla
  MOD_RES (14)..(14): x is bAla
RXRRXRRFQILYRXRR

SEQ ID NO: 29 - protein, 17 aa, Artificial Sequence
  peptide
  MOD_RES (2)..(2): x is bAla
  MOD_RES (5)..(5): x is bAla
  MOD_RES (14)..(14): x is bAla
  MOD_RES (16)..(16): x is bAla
RXRRXRFQILYRRXRXR

SEQ ID NO: 30 - protein, 17 aa, Artificial Sequence
  peptide
  MOD_RES (2)..(2): x is bAla
  MOD_RES (4)..(4): x is bAla
  MOD_RES (12)..(12): x is bAla
  MOD_RES (15)..(15): x is bAla
RXRXRFQILYRXRRXRR

SEQ ID NO: 31 - protein, 17 aa, Artificial Sequence
  peptide
  MOD_RES (2)..(2): x is bAla
  MOD_RES (5)..(5): x is bAla
  MOD_RES (14)..(14): x is bAla
  MOD_RES (16)..(16): x is bAla
RXRRXRRYQFLIRXRXR

SEQ ID NO: 32 - protein, 17 aa, Artificial Sequence
  peptide
  MOD_RES (2)..(2): x is bAla
  MOD_RES (5)..(5): x is bAla
  MOD_RES (14)..(14): x is bAla
  MOD_RES (16)..(16): x is bAla
RXRRXRRILFQYRXRXR

SEQ ID NO: 33 - protein, 16 aa, Artificial Sequence
  peptide
  MOD_RES (2)..(2): x is bAla
  MOD_RES (5)..(5): x is bAla
  MOD_RES (13)..(13): x is bAla
  MOD_RES (15)..(15): x is bAla
RXRRXRFQILYRXRXR

SEQ ID NO: 34 - protein, 16 aa, Artificial Sequence
  peptide
  MOD_RES (2)..(2): x is bAla
  MOD_RES (5)..(5): x is bAla
  MOD_RES (12)..(12): x is bAla
  MOD_RES (15)..(15): x is bAla
RXRRXFQILYRXRRXR

SEQ ID NO: 35 - protein, 15 aa, Artificial Sequence
  peptide
  MOD_RES (2)..(2): x is bAla
  MOD_RES (5)..(5): x is bAla
  MOD_RES (12)..(12): x is bAla
  MOD_RES (14)..(14): x is bAla
RXRRXRFQILYXRXR

SEQ ID NO: 36 - protein, 15 aa, Artificial Sequence
  peptide
  MOD_RES (2)..(2): x is bAla
  MOD_RES (5)..(5): x is bAla
  MOD_RES (12)..(12): x is bAla
  MOD_RES (14)..(14): x is bAla
RXRRXFQILYRXRXR

SEQ ID NO: 37 - protein, 17 aa, Artificial Sequence
  peptide
  MOD_RES (2)..(2): x is bAla
  MOD_RES (5)..(5): x is bAla
  MOD_RES (14)..(14): x is bAla
  MOD_RES (16)..(16): x is bAla
RXRRXRRFQILYRXHXH

SEQ ID NO: 38 - protein, 17 aa, Artificial Sequence
  peptide
  MOD_RES (2)..(2): x is bAla
  MOD_RES (5)..(5): x is bAla
  MOD_RES (14)..(14): x is bAla
  MOD_RES (16)..(16): x is bAla
RXRRXRRFQILYHXHXR

SEQ ID NO: 39 - protein, 17 aa, Artificial Sequence
  peptide
  MOD_RES (2)..(2): x is bAla
  MOD_RES (5)..(5): x is bAla
  MOD_RES (14)..(14): x is bAla
  MOD_RES (16)..(16): x is bAla
RXRRXRRFQILYHXRXH

SEQ ID NO: 40 - protein, 17 aa, Artificial Sequence
  peptide
  MOD_RES (2)..(2): x is bAla
  MOD_RES (5)..(5): x is bAla
  MOD_RES (14)..(14): x is bAla
  MOD_RES (16)..(16): x is bAla
RXRRXRRYQFLIRXHXH

SEQ ID NO: 41 - protein, 17 aa, Artificial Sequence
  peptide
  MOD_RES (2)..(2): x is bAla
  MOD_RES (5)..(5): x is bAla
  MOD_RES (14)..(14): x is bAla
  MOD_RES (16)..(16): x is bAla
RXRRXRRILFQYRXHXH

SEQ ID NO: 42 - protein, 17 aa, Artificial Sequence
  peptide
  MOD_RES (2)..(2): x is bAla
  MOD_RES (5)..(5): x is bAla
  MOD_RES (14)..(14): x is bAla
  MOD_RES (16)..(16): x is bAla
RXRHXHRFQILYRXRXR

SEQ ID NO: 43 - protein, 17 aa, Artificial Sequence
  peptide
  MOD_RES (2)..(2): x is bAla
  MOD_RES (4)..(4): x is bAla
  MOD_RES (5)..(5): x is bAla
  MOD_RES (14)..(14): x is bAla
  MOD_RES (16)..(16): x is bAla
RXRXXHRFQILYRXHXH

SEQ ID NO: 44 - protein, 16 aa, Artificial Sequence
  peptide
  MOD_RES (2)..(2): x is bAla
  MOD_RES (5)..(5): x is bAla
  MOD_RES (13)..(13): x is bAla
  MOD_RES (15)..(15): x is bAla
RXRRXRFQILYRXHXH

SEQ ID NO: 45 - protein, 16 aa, Artificial Sequence
  peptide
  MOD_RES (2)..(2): x is bAla
  MOD_RES (5)..(5): x is bAla
  MOD_RES (13)..(13): x is bAla
  MOD_RES (15)..(15): x is bAla
RXRRXRFQILYHXHXH

SEQ ID NO: 46 - protein, 16 aa, Artificial Sequence
  peptide
  MOD_RES (2)..(2): x is bAla
  MOD_RES (5)..(5): x is bAla
  MOD_RES (13)..(13): x is bAla
  MOD_RES (15)..(15): x is bAla
RXRRXHFQILYRXHXH

SEQ ID NO: 47 - protein, 16 aa, Artificial Sequence
  peptide
  MOD_RES (2)..(2): x is bAla
  MOD_RES (5)..(5): x is bAla
  MOD_RES (13)..(13): x is bAla
  MOD_RES (15)..(15): x is bAla
HXRRXRFQILYRXHXH

SEQ ID NO: 48 - protein, 15 aa, Artificial Sequence
  peptide
  MOD_RES (2)..(2): x is bAla
  MOD_RES (5)..(5): x is bAla
  MOD_RES (12)..(12): x is bAla
  MOD_RES (14)..(14): x is bAla
RXRRXFQILYRXHXH

SEQ ID NO: 49 - protein, 15 aa, Artificial Sequence
  peptide
  MOD_RES (2)..(2): x is bAla
  MOD_RES (5)..(5): x is bAla
  MOD_RES (12)..(12): x is bAla
  MOD_RES (14)..(14): x is bAla
RXRRXRFQILYXHXH

SEQ ID NO: 50 - protein, 16 aa, Artificial Sequence
  peptide
  MOD_RES (2)..(2): x is bAla
  MOD_RES (5)..(5): x is bAla
  MOD_RES (13)..(13): x is bAla
  MOD_RES (15)..(15): x is bAla
RXRRXRYQFLIHXHXH

SEQ ID NO: 51 - protein, 16 aa, Artificial Sequence
  peptide
  MOD_RES (2)..(2): x is bAla
  MOD_RES (5)..(5): x is bAla
  MOD_RES (13)..(13): x is bAla
  MOD_RES (15)..(15): x is bAla
RXRRXRILFQYHXHXH

SEQ ID NO: 52 - protein, 17 aa, Artificial Sequence
  peptide
  MOD_RES (2)..(2): x is bAla
  MOD_RES (5)..(5): x is bAla
  MOD_RES (14)..(14): x is bAla
  MOD_RES (16)..(16): x is bAla
RXRRXRRFQILYHXHXH

SEQ ID NO: 53 - protein, 15 aa, Artificial Sequence
  peptide
  MOD_RES (2)..(2): x is bAla
  MOD_RES (5)..(5): x is bAla
  MOD_RES (12)..(12): x is bAla
  MOD_RES (14)..(14): x is bAla
RXRRXRFQILYXRXS

SEQ ID NO: 54 - protein, 15 aa, Artificial Sequence
  peptide
  MOD_RES (2)..(2): x is bAla
  MOD_RES (5)..(5): x is bAla
  MOD_RES (12)..(12): x is bAla
  MOD_RES (14)..(14): x is bAla
  MOD_RES (15)..(15): x hydroxyproline
RXRRXRFQILYXRXX

SEQ ID NO: 55 - protein, 15 aa, Artificial Sequence
  peptide
  MOD_RES (2)..(2): x is bAla
  MOD_RES (5)..(5): x is bAla
  MOD_RES (12)..(12): x is bAla
  MOD_RES (14)..(14): x is hydroxyproline
RXRRXRFQILYXRXR

SEQ ID NO: 56 - protein, 16 aa, Artificial Sequence
  peptide
  MOD_RES (3)..(3): x is bAla
  MOD_RES (6)..(6): x is bAla
  MOD_RES (13)..(13): x is bAla
  MOD_RES (15)..(15): x is bAla
RRXRRXRFQILYXRXR

SEQ ID NO: 57 - protein, 15 aa, Artificial Sequence
  peptide
  MOD_RES (1)..(1): x is bAla
  MOD_RES (4)..(4): x is bAla
  MOD_RES (12)..(12): x is bAla
  MOD_RES (14)..(14): x is bAla
XRRXRRFQILYXRXR

SEQ ID NO: 58 - protein, 13 aa, Artificial Sequence
  peptide
  MOD_RES (2)..(2): x is bAla
  MOD_RES (5)..(5): x is bAla
  MOD_RES (10)..(10): x is bAla
  MOD_RES (12)..(12): x is bAla
RXRRXRWWWXRXR

SEQ ID NO: 59 - protein, 15 aa, Artificial Sequence
  peptide
  MOD_RES (2)..(2): x is bAla
  MOD_RES (5)..(5): x is bAla
  MOD_RES (12)..(12): x is bAla
  MOD_RES (14)..(14): x is bAla
RXRRXRWWPWWXRXR

SEQ ID NO: 60 - protein, 14 aa, Artificial Sequence
  peptide
  MOD_RES (2)..(2): x is bAla
  MOD_RES (5)..(5): x is bAla
  MOD_RES (11)..(11): x is bAla
  MOD_RES (13)..(13): x is bAla
RXRRXRWPWWXRXR

SEQ ID NO: 61 - protein, 14 aa, Artificial Sequence
  peptide
  MOD_RES (2)..(2): x is bAla
  MOD_RES (5)..(5): x is bAla
  MOD_RES (11)..(11): x is bAla
  MOD_RES (13)..(13): x is bAla
RXRRXRWWPWXRXR

SEQ ID NO: 62 - protein, 15 aa, Artificial Sequence
  peptide
  MOD_RES (2)..(2): x is bAla
  MOD_RES (5)..(5): x is bAla
  MOD_RES (12)..(12): x is bAla
  MOD_RES (14)..(14): x is bAla
RXRRXRRWWWRXRXR

SEQ ID NO: 63 - protein, 17 aa, Artificial Sequence
  peptide
  MOD_RES (2)..(2): x is bAla
  MOD_RES (5)..(5): x is bAla
  MOD_RES (14)..(14): x is bAla
  MOD_RES (16)..(16): x is bAla
RXRRXRRWWPWWRXRXR

SEQ ID NO: 64 - protein, 16 aa, Artificial Sequence
  peptide
  MOD_RES (2)..(2): x is bAla
  MOD_RES (5)..(5): x is bAla
  MOD_RES (13)..(13): x is bAla
  MOD_RES (15)..(15): x is bAla
RXRRXRRWPWWRXRXR

SEQ ID NO: 65 - protein, 16 aa, Artificial Sequence
  peptide
  MOD_RES (2)..(2): x is bAla
  MOD_RES (5)..(5): x is bAla
  MOD_RES (13)..(13): x is bAla
  MOD_RES (15)..(15): x is bAla
RXRRXRRWWPWRXRXR

SEQ ID NO: 66 - protein, 16 aa, Artificial Sequence
  peptide
  MOD_RES (2)..(2): x is bAla
  MOD_RES (5)..(5): x is bAla
  MOD_RES (13)..(13): x is bAla
  MOD_RES (15)..(15): x is bAla
RXRRXRRFQILYXRXR

SEQ ID NO: 67 - protein, 15 aa, Artificial Sequence
  peptide
  MOD_RES (2)..(2): x is bAla
  MOD_RES (5)..(5): x is bAla
  MOD_RES (14)..(14): x is bAla
RXRRXRRFQILYRXR

SEQ ID NO: 68 - protein, 16 aa, Artificial Sequence
  peptide
  MOD_RES (1)..(1): x is bAla
  MOD_RES (3)..(3): x is bAla
  MOD_RES (5)..(5): x is bAla
  MOD_RES (12)..(12): x is bAla
  MOD_RES (15)..(15): x is bAla
XRXRXWWPWWRXRRXR

SEQ ID NO: 69 - protein, 16 aa, Artificial Sequence
  peptide
  MOD_RES (2)..(2): x is bAla
  MOD_RES (5)..(5): x is bAla
  MOD_RES (13)..(13): x is bAla
  MOD_RES (15)..(15): x is bAla
RXRRXRRFQILYXHXH

SEQ ID NO: 70 - protein, 16 aa, Artificial Sequence
  peptide
  MOD_RES (2)..(2): x is bAla
  MOD_RES (5)..(5): x is bAla
  MOD_RES (13)..(13): x is bAla
  MOD_RES (15)..(15): x is bAla
RXRRXRRFQIYRXHXH

SEQ ID NO: 71 - protein, 15 aa, Artificial Sequence
  peptide
  MOD_RES (2)..(2): x is bAla
  MOD_RES (5)..(5): x is bAla
  MOD_RES (12)..(12): x is bAla
  MOD_RES (14)..(14): x is bAla
RXRRXRFQILYXRXH

SEQ ID NO: 72 - protein, 16 aa, Artificial Sequence
  peptide
  MOD_RES (2)..(2): x is bAla
  MOD_RES (5)..(5): x is bAla
  MOD_RES (13)..(13): x is hydroxyproline
  MOD_RES (15)..(15): x is hydroxyproline
RXRRXRFQILYRXHXH

SEQ ID NO: 73 - protein, 16 aa, Artificial Sequence
  peptide
  MOD_RES (2)..(2): x is hydroxyproline
  MOD_RES (5)..(5): x is hydroxyproline
  MOD_RES (13)..(13): x is bAla
  MOD_RES (15)..(15): x is bAla
RXRRXRFQILYRXHXH

SEQ ID NO: 74 - protein, 16 aa, Artificial Sequence
  peptide
  MOD_RES (2)..(2): x is hydroxyproline
  MOD_RES (5)..(5): x is hydroxyproline
  MOD_RES (13)..(13): x is hydroxyproline
  MOD_RES (15)..(15): x is hydroxyproline
RXRRXRFQILYRXHXH

SEQ ID NO: 75 - protein, 14 aa, Artificial Sequence
  peptide
  MOD_RES (2)..(2): x is bAla
  MOD_RES (5)..(5): x is bAla
  MOD_RES (11)..(11): x is bAla
  MOD_RES (13)..(13): x is bAla
RXRRXRWWWRXHXH

SEQ ID NO: 76 - protein, 14 aa, Artificial Sequence
  peptide
  MOD_RES (2)..(2): x is bAla
  MOD_RES (5)..(5): x is bAla
  MOD_RES (11)..(11): x is bAla
  MOD_RES (13)..(13): x is bAla
RXRRXRWWPRXHXH

SEQ ID NO: 77 - protein, 14 aa, Artificial Sequence
  peptide
  MOD_RES (2)..(2): x is bAla
  MOD_RES (5)..(5): x is bAla
  MOD_RES (11)..(11): x is bAla
  MOD_RES (13)..(13): x is bAla
RXRRXRPWWRXHXH

SEQ ID NO: 78 - protein, 16 aa, Artificial Sequence
  peptide
  MOD_RES (2)..(2): x is bAla
  MOD_RES (5)..(5): x is bAla
  MOD_RES (13)..(13): x is bAla
  MOD_RES (15)..(15): x is bAla
RXRRXRWWPWWRXHXH

SEQ ID NO: 79 - protein, 15 aa, Artificial Sequence
  peptide
  MOD_RES (2)..(2): x is bAla
  MOD_RES (5)..(5): x is bAla
  MOD_RES (12)..(12): x is bAla
  MOD_RES (14)..(14): x is bAla
RXRRXRWWPWRXHXH

SEQ ID NO: 80 - protein, 15 aa, Artificial Sequence
  peptide
  MOD_RES (2)..(2): x is bAla
  MOD_RES (5)..(5): x is bAla
  MOD_RES (12)..(12): x is bAla
  MOD_RES (14)..(14): x is bAla
RXRRXRWPWWRXHXH

SEQ ID NO: 81 - protein, 15 aa, Artificial Sequence
  peptide
  MOD_RES (2)..(2): x is bAla
  MOD_RES (5)..(5): x is bAla
  MOD_RES (12)..(12): x is bAla
  MOD_RES (14)..(14): x is bAla
RXRRXRRWWWRXHXH

SEQ ID NO: 82 - protein, 17 aa, Artificial Sequence
  peptide
  MOD_RES (2)..(2): x is bAla
  MOD_RES (5)..(5): x is bAla
  MOD_RES (14)..(14): x is bAla
  MOD_RES (16)..(16): x is bAla
RXRRXRRWWPWWRXHXH

SEQ ID NO: 83 - protein, 16 aa, Artificial Sequence
  peptide
  MOD_RES (2)..(2): x is bAla
  MOD_RES (5)..(5): x is bAla
  MOD_RES (13)..(13): x is bAla
  MOD_RES (15)..(15): x is bAla
RXRRXRRWPWWRXHXH

SEQ ID NO: 84 - protein, 16 aa, Artificial Sequence
  peptide
  MOD_RES (2)..(2): x is bAla
  MOD_RES (5)..(5): x is bAla
  MOD_RES (13)..(13): x is bAla
  MOD_RES (15)..(15): x is bAla
RXRRXRRWWPWRXHXH

SEQ ID NO: 85 - protein, 17 aa, Artificial Sequence
  peptide
  MOD_RES (3)..(3): x is bAla
  MOD_RES (6)..(6): x is bAla
  MOD_RES (14)..(14): x is bAla
  MOD_RES (16)..(16): x is bAla
RRXRRXRFQILYRXHXH

SEQ ID NO: 86 - protein, 16 aa, Artificial Sequence
  peptide
  MOD_RES (1)..(1): x is bAla
  MOD_RES (4)..(4): x is bAla
  MOD_RES (13)..(13): x is bAla
  MOD_RES (15)..(15): x is bAla
XRRXRRFQILYRXHXH

SEQ ID NO: 87 - protein, 16 aa, Artificial Sequence
  peptide
  MOD_RES (3)..(3): x is bAla
  MOD_RES (6)..(6): x is bAla
  MOD_RES (13)..(13): x is bAla
  MOD_RES (15)..(15): x is bAla
RRXRRXRFQILYXHXH

SEQ ID NO: 88 - protein, 15 aa, Artificial Sequence
  peptide
  MOD_RES (1)..(1): x is bAla
  MOD_RES (4)..(4): x is bAla
  MOD_RES (12)..(12): x is bAla
  MOD_RES (14)..(14): x is bAla
XRRXRRFQILYXHXH

SEQ ID NO: 89 - protein, 17 aa, Artificial Sequence
  peptide
  MOD_RES (2)..(2): x is bAla
  MOD_RES (5)..(5): x is bAla
  MOD_RES (14)..(14): x is bAla
  MOD_RES (16)..(16): x is bAla
RXRRXHRFQILYRXHXH

SEQ ID NO: 90 - protein, 15 aa, Artificial Sequence
  peptide
  MISC_FEATURE (2)..(2): x is bAla
  MISC_FEATURE (5)..(5): x is bAla
  MISC_FEATURE (12)..(12): x is hydroxyproline
  MISC_FEATURE (14)..(14): x is hydroxyproline
RXRRXRFQILYXRXR

SEQ ID NO: 91 - protein, 15 aa, Artificial Sequence
  peptide
  MISC_FEATURE (2)..(2): x is hydroxyproline
  MISC_FEATURE (5)..(5): x is hydroxyproline
  MISC_FEATURE (12)..(12): x is bAla
  MISC_FEATURE (14)..(14): x is bAla
RXRRXRFQILYXRXR

SEQ ID NO: 92 - protein, 15 aa, Artificial Sequence
  peptide
  MISC_FEATURE (2)..(2): x is hydroxyproline
  MISC_FEATURE (5)..(5): x is hydroxyproline
  MISC_FEATURE (12)..(12): x is hydroxyproline
  MISC_FEATURE (14)..(14): x is hydroxyproline
RXRRXRFQILYXRXR

SEQ ID NO: 93 - protein, 13 aa, Artificial Sequence
  peptide
  MISC_FEATURE (2)..(2): x is bAla
  MISC_FEATURE (5)..(5): x is bAla
  MISC_FEATURE (10)..(10): x is bAla
  MISC_FEATURE (12)..(12): x is bAla
RXRRXRWWWXRXR

SEQ ID NO: 94 - protein, 15 aa, Artificial Sequence
  peptide
  MISC_FEATURE (2)..(2): x is bAla
  MISC_FEATURE (5)..(5): x is bAla
  MISC_FEATURE (12)..(12): x is bAla
  MISC_FEATURE (14)..(14): x is bAla
RXRRXRWWPWWXRXR

SEQ ID NO: 95 - DNA, 21 bp, Artificial Sequence
  21-mer PMO antisense sequence
cagcagcagcagcagcagcag

SEQ ID NO: 96 - DNA, 21 bp, Artificial Sequence
  phosphorothioate probe
  misc_feature (1)..(1): labelled with digoxigenin
  misc_feature (21)..(21): labelled with biotin
ctgctgctgctgctgctgctg

SEQ ID NO: 97 - protein, 11 aa, Artificial Sequence
  D-PEP 5.70
  MISC_FEATURE (2)..(2): x is bAla
  MISC_FEATURE (4)..(4): x is bAla
  MISC_FEATURE (6)..(6): glucosylated serine residue
  MISC_FEATURE (8)..(8): x is bAla
  MISC_FEATURE (10)..(10): x is bAla
RXRXRSRXRXR

SEQ ID NO: 98 - protein, 21 aa, Artificial Sequence
  Pip6a
  MISC_FEATURE (2)..(2): x is aminohexanoic acid
  MISC_FEATURE (5)..(5): x is bAla
  MISC_FEATURE (8)..(8): x is aminohexanoic acid
  MISC_FEATURE (16)..(16): x is aminohexanoic acid
  MISC_FEATURE (18)..(18): x is bAla
  MISC_FEATURE (20)..(20): x is aminohexanoic acid
RXRRXRRXRYQFLIRXRXRXR

SEQ ID NO: 99 - protein, 17 aa, Artificial Sequence
  Pip9b2
  MISC_FEATURE (2)..(2): x is aminohexanoic acid
  MISC_FEATURE (5)..(5): x is bAla
  MISC_FEATURE (14)..(14): x is bAla
  MISC_FEATURE (16)..(16): x is aminohexanoic acid
RXRRXRRFQILYRXRXR

SEQ ID NO: 100 - DNA, 21 bp, Artificial Sequence
  primer Mbnl1.F
gctgcccaataccaggtcaac

SEQ ID NO: 101 - DNA, 22 bp, Artificial Sequence
  primer Mbnl1.R
tggtgggagaaatgctgtatgc

SEQ ID NO: 102 - DNA, 22 bp, Artificial Sequence
  primer Clcn1.F
ttcacatcgccagcatctgtgc

SEQ ID NO: 103 - DNA, 25 bp, Artificial Sequence
  primer Clcn1.R
cacggaacacaaaggcactgaatgt

SEQ ID NO: 104 - DNA, 23 bp, Artificial Sequence
  primer Serca.F
gctcatggtcctcaagatctcac

SEQ ID NO: 105 - DNA, 20 bp, Artificial Sequence
  primer Serca.R
gggtcagtgcctcagctttg

SEQ ID NO: 106 - DNA, 22 bp, Artificial Sequence
  primer Ldb3.F
ggaagatgaggctgatgagtgg

SEQ ID NO: 107 - DNA, 24 bp, Artificial Sequence
  primer Ldb3.R
tgctgacagtggtagtgctctttc

SEQ ID NO: 108 - DNA, 21 bp, Artificial Sequence
  primer BIN.F
agaacctcaatgatgtgctgg

SEQ ID NO: 109 - DNA, 21 bp, Artificial Sequence
  primer BIN.R
tcgtgttgactctgatctcgg

SEQ ID NO: 110 - DNA, 20 bp, Artificial Sequence
  primer DMD.F
ttagaggaggtgatggagca

SEQ ID NO: 111 - DNA, 20 bp, Artificial Sequence
  primer DMD.R
gatactaaggactccatcgc

SEQ ID NO: 112 - DNA, 20 bp, Artificial Sequence
  primer INSR.F
ccaaagacagactctcagat

SEQ ID NO: 113 - DNA, 20 bp, Artificial Sequence
  primer INSR.R
aacatcgccaagggacctgc

SEQ ID NO: 114 - DNA, 23 bp, Artificial Sequence
  primer LDB3.F
gcaagaccctgatgaagaagctc

SEQ ID NO: 115 - DNA, 19 bp, Artificial Sequence
  primer LDB3.R
gacagaaggccggatgctg

SEQ ID NO: 116 - DNA, 20 bp, Artificial Sequence
  primer SERCA.F
atcttcaagctccgggccct

SEQ ID NO: 117 - DNA, 20 bp, Artificial Sequence
  primer SERCA.R
cagctctgcctgaagatgtg

SEQ ID NO: 118 - DNA, 23 bp, Artificial Sequence
  primer SOS1.F
cagtaccacagatgtttgcagtg

SEQ ID NO: 119 - DNA, 22 bp, Artificial Sequence
  primer SOS1.R
tctggtcgtcttcgtggaggaa

SEQ ID NO: 120 - DNA, 24 bp, Artificial Sequence
  primer TNNT2.F
atagaagaggtggtggaagagtac

SEQ ID NO: 121 - DNA, 24 bp, Artificial Sequence
  primer TNNT2.R
gtctcagcctctgcttcagcatcc